SEQUENCE LISTING (3 sequences)

SEQ ID NO: 1 - RNA, 10 nt, synthetic construct
  source 1..10: mol_type = other RNA
ccrrccatgg

SEQ ID NO: 2 - RNA, 96 nt, synthetic construct
  source 1..96: mol_type = other RNA
ggctcatctcgacaagaggcggcagtcctcagtactcttactcttttctgtaaagaggagactgctggactcgccgcccaagttcgagcatgagcc

SEQ ID NO: 3 - RNA, 74 nt, synthetic construct
  source 1..74: mol_type = other RNA
ggctagaggcggcagtcctcagtactcttactcttttctgtaaagaggagactgctggactcgccgcccgagcc

(19) United States
(12) Patent Application Publication
(10) Pub. No.: US 2025/0051386 A1
(43) Pub. Date: Feb. 13, 2025

(54) COMPOSITIONS AND METHODS FOR PURIFYING POLYRIBONUCLEOTIDES

(71) Applicant: Flagship Pioneering Innovations VI, LLC, Cambridge, MA (US)

(72) Inventors: Alexandra Sophie DE BOER, Somerville, MA (US); Nicholas McCartney PLUGIS, Duxbury, MA (US); Catherine CIFUENTES-ROJAS, Brookline, MA (US); Ki Young PAEK, Brighton, MA (US); Elissa Magdalene HOBERT, Cambridge, MA (US); Joshua Nathan FARB, Somerville, MA (US); Vadim DUDKIN, Wellesley, MA (US)

(21) Appl. No.: 18/723,239

(22) PCT Filed: Dec. 22, 2022
(86) PCT No.: PCT/US2022/082265

§ 371 Date: Jun. 21, 2024

Related U.S. Application Data
(60) Provisional application No. 63/292,557, filed on Dec. 22, 2021.

(51) Int. Cl. C07H 21/02; A61K 31/7105; C07H 1/08; C12N 15/10

(52) U.S. Cl. CPC C07H 21/02; A61K 31/7105; C07H 1/08; C12N 15/1013

(57) ABSTRACT

The present disclosure relates to compositions and methods for separating and/or purifying polyribonucleotides. The polyribonucleotide may be separated from a mixture of polyribonucleotides with an oligonucleotide that hybridizes to a target region of the polyribonucleotide.

### SEQUENCE LISTING

This application contains a Sequence Listing which has been filed electronically in Extensible Markup Language (XML) format and is hereby incorporated by reference in its entirety. Said XML copy, created on Dec. 22, 2022, is named 51509-056WO2_Sequence_Listing_12_21_22.XML and is 3,683 bytes in size.

### BACKGROUND

Polyribonucleotides are useful for a variety of therapeutic and engineering applications. Thus, new compositions and methods for separating and purifying polyribonucleotides are needed.

### SUMMARY OF THE INVENTION

In one aspect, the disclosure features a method of separating a linear polyribonucleotide from a plurality of polyribonucleotides. The plurality of polyribonucleotides include a mixture of linear polyribonucleotides and circular polyribonucleotides that include an open reading frame (ORF) encoding a polypeptide. The method includes (a) providing a sample with the plurality of polyribonucleotides, wherein a subset of the plurality of polyribonucleotides include the linear polyribonucleotide; (b) attaching a target region to the linear polyribonucleotide; and (c) contacting the sample with an oligonucleotide that hybridizes to the target region. The method further includes (d) separating the linear polyribonucleotide with the target region that is hybridized to the oligonucleotide from the plurality of polyribonucleotides in the sample.

In some embodiments, the oligonucleotide is conjugated (e.g., directly or indirectly) to a particle. The particle may be, for example, a magnetic bead. In some embodiments, the oligonucleotide is conjugated to a resin that includes a plurality of the particles. The resin may include, for example, cross-linked poly[styrene-divinylbenzene], agarose, or SEPHAROSE® agarose. In some embodiments, a column includes the resin.

In some embodiments, separating the linear polyribonucleotide includes immobilizing the oligonucleotide.

In some embodiments, separating the linear polyribonucleotide includes collecting a portion of the sample that is not hybridized to the oligonucleotide. For example, the portion of the sample that is not hybridized to the oligonucleotide may include the circular polyribonucleotide.

In some embodiments, a level of expression from the ORF of the circular polyribonucleotide after purification is increased at least 10% relative to a level of expression from the ORF prior to purification.

In another aspect, the disclosure features a method of separating a linear polyribonucleotide from a plurality of polyribonucleotides. The plurality of polyribonucleotides include a mixture of linear polyribonucleotides and circular polyribonucleotides. The method includes (a) providing a sample with the plurality of polyribonucleotides, wherein a subset of the plurality of polyribonucleotides include the linear polyribonucleotide; (b) attaching a target region to the linear polyribonucleotide; and (c) contacting the sample with a column that includes a resin with a plurality of particles conjugated to an oligonucleotide that hybridizes to the target region. The method further includes (d) collecting an eluate that includes a portion of the sample that is not hybridized to the oligonucleotide from the plurality of polyribonucleotides in the sample.

In some embodiments, the portion of the sample that is not hybridized to the oligonucleotide includes the circular polyribonucleotide.

In some embodiments, the resin includes cross-linked poly[styrene-divinylbenzene], agarose, or SEPHAROSE® agarose.

In some embodiments of any of the above aspects, the method includes a step of circularizing the circular polyribonucleotide from a linear precursor prior to step (a). For example, the linear precursor may include a 5′ self-splicing intron fragment and a 3′ self-splicing intron fragment, and the circular polyribonucleotide is produced by self-splicing of the linear precursor. The 5′ self-splicing intron fragment and the 3′ self-splicing intron fragment may each be, for example, a Group I or Group II self-splicing intron fragment.

In another aspect, the disclosure features a method of separating a linear polyribonucleotide from a plurality of polyribonucleotides. The plurality of polyribonucleotides include a mixture of linear polyribonucleotides and circular polyribonucleotides. The method includes (a) circularizing a linear precursor to form the circular polyribonucleotide; (b) providing a sample that includes the plurality of polyribonucleotides, wherein a subset of the plurality of polyribonucleotides include the linear polyribonucleotide; (c) attaching a target region to the linear polyribonucleotide; and (d) contacting the sample with an oligonucleotide that hybridizes to the target region. The method further includes (e) separating the linear polyribonucleotide with the target region that is hybridized to the oligonucleotide from the plurality of polyribonucleotides in the sample.

In some embodiments, circularizing the circular polyribonucleotide is produced by splint-ligation of the linear precursor.

In some embodiments, the linear precursor includes a 5′ self-splicing intron fragment and a 3′ self-splicing intron fragment, and the circular polyribonucleotide is produced by self-splicing of the linear precursor.

In some embodiments, the 5′ self-splicing intron fragment and the 3′ self-splicing intron fragment are each a Group I or Group II self-splicing intron fragment.

In some embodiments of any of the above aspects, the circular polyribonucleotide includes an ORF. The ORF may encode, for example, a polypeptide.

In some embodiments of any of the above aspects, the circular polyribonucleotide includes an internal ribosome entry site (IRES). The ORF may be operably linked to the IRES.

In some embodiments, the method includes attaching the target region to a 3′ or 5′ terminus of the linear polyribonucleotide. For example, the method may include attaching the target region to the 3′ terminus of the linear polyribonucleotide. The attaching step may include polyadenylating the 3′ terminus of the linear polyribonucleotide. The polyadenylation may include providing a polyA polymerase, e.g., E. coli polyA polymerase. In another embodiment, the attaching step includes ligating the target region to the 3′ terminus of the linear polyribonucleotide.

In some embodiments, the linear polyribonucleotide of step (a) comprises a target region (e.g., prior to attaching the target region in step (b)). Accordingly, such embodiments provide a further method of purification wherein a first copy of a target region is present in the linear polyribonucleotide prior to step (b), and a second copy of the target region is attached to the linear polyribonucleotide during step (b). The target region present in the linear polyribonucleotide of step (a) may be the same or different (e.g., in sequence and length) than the target region attached to the linear polyribonucleotide in step (b). In some embodiments, the linear precursor includes, operably linked in the following 5′ to 3′ order: a target region, a first circularization element (e.g., a first self-splicing intron fragment), a polyribonucleotide cargo (e.g., optionally including one or more of a spacer, an IRES, and an open reading frame), and a second circularization element (e.g., a second self-splicing intron fragment).

In some embodiments, the circular polyribonucleotide does not include a polyA sequence (e.g., a polyA sequence of at least 10, e.g., at least 11, 12, 13, 14, 15, 16, 17, 18, 19, 20, 21, 22, 23, 24, or 25, e.g., at least 15, at least 20, at least 25 or at least 30) contiguous adenosine nucleotides.

In some embodiments, the target region includes a polyA sequence (e.g., a polyA sequence of at least 10 contiguous adenosine nucleotides).

In some embodiments, the oligonucleotide that binds the target includes a polyU or polydT sequence. The polyU or polydT sequence may include at least 10 (e.g., at least 11, 12, 13, 14, 15, 16, 17, 18, 19, 20, 21, 22, 23, 24, 25, e.g., at least 15, at least 20, at least 25 or at least 30) uridine or thymidine residues. In some embodiments, the polydT includes (dT)₂₅.

In some embodiments, the oligonucleotide is conjugated to a particle. The particle may be, for example, a magnetic bead. In some embodiments, the oligonucleotide is conjugated to a resin that includes a plurality of the particles. The resin may include, for example, cross-linked poly[styrene-divinylbenzene], agarose, or SEPHAROSE® agarose. In some embodiments, a column includes the resin.

In some embodiments, separating the linear polyribonucleotide includes immobilizing the oligonucleotide.

In some embodiments, separating the linear polyribonucleotide includes collecting a portion of the sample that is not hybridized to the oligonucleotide. For example, the portion of the sample that is not hybridized to the oligonucleotide may include the circular polyribonucleotide.

In some embodiments of any of the above aspects, the method further includes washing the linear polyribonucleotide with the target region that is hybridized to the oligonucleotide one or more times.

In some embodiments of any of the above aspects, the method further includes eluting the linear polyribonucleotide with the target region from the oligonucleotide.

In some embodiments, the method includes providing a plurality of oligonucleotides, wherein each oligonucleotide hybridizes to a distinct target region.

In some embodiments, the oligonucleotide has at least 5 nucleotides (e.g., at least 6, 7, 8, 9, 10, 11, 12, 13, 14, 15, 16, 17, 18, 19, 20, 21, 22, 23, 24, 25, 26, 27, 28, 29, 30, 35, 40, 45, 50, 55, 60, 65, 70, 75, 80, 85, 90, 95, 100, or more nucleotides) in length. In some embodiments, the oligonucleotide is, e.g., from 5-100, 5-95, 10-90, 10-80, 12-60, 15-50, 15-40, 15-30, 18-30, 20-25, or 20-22 nucleotides in length. In some embodiments, the oligonucleotide is 23 nucleotides in length. In some embodiments, the oligonucleotide has at least 80% (e.g., at least 85%, 90%, 95%, 97%, 99%, or 100%) complementarity an equal length portion of the target region.

In some embodiments, the method includes providing the oligonucleotide at a molar ratio of 10:1 to 1:10 to the linear polyribonucleotide with the target region.

In some embodiments, a level of expression from the ORF of the circular polyribonucleotide after purification is increased at least 10% relative to a level of expression from the ORF prior to purification.

In some embodiments of any of the above aspects, the method separates at least 500 μg (e.g., at least 600 μg, 700 μg, 800 μg, 900 μg, 1 mg, 2 mg, 3 mg, 4 mg, 5 mg, 6 mg, 7 mg, 8 mg, 9 mg, 10 mg, 20 mg, 30 mg, 40 mg, 50 mg, 60 mg, 70 mg, 80 mg, 90 mg, 100 mg, 200 mg, 300 mg, 400 mg, 500 mg, 600 mg, 700 mg, 800 mg, 900 mg, 1,000 mg, or more) of the linear polyribonucleotide with the target region. In some embodiments, the method separates from 500 μg to 1,000 mg of the linear polyribonucleotide including the target region.

In another aspect, the disclosure features a population of polyribonucleotides produced by the method as described herein (e.g., of any of the aspects).

In some embodiments, the population includes a circular polyribonucleotide lacking the target region and the circular polyribonucleotide includes at least 40% (e.g., at least 50%, 60%, 70%, 80%, 855, 90%, 95%, 97%, 99%, or 100%) (mol/mol) of the total polyribonucleotides in the composition.

In some embodiments, the population includes less than 40% (e.g., less than 35%, 30%, 25%, 20%, 15%, 10%, 5%, 4%, 3%, 2%, or 1%) (mol/mol) linear polyribonucleotides of the total polyribonucleotides in the composition.

In some embodiments, a total weight of polyribonucleotides in the population of polyribonucleotides at least 500 μg (e.g., at least 600 μg, 700 μg, 800 μg, 900 μg, 1 mg, 2 mg, 3 mg, 4 mg, 5 mg, 6 mg, 7 mg, 8 mg, 9 mg, 10 mg, 20 mg, 30 mg, 40 mg, 50 mg, 60 mg, 70 mg, 80 mg, 90 mg, 100 mg, 200 mg, 300 mg, 400 mg, 500 mg, 600 mg, 700 mg, 800 mg, 900 mg, 1,000 mg, or more). In some embodiments, the total weight of polyribonucleotides in the population of polyribonucleotides is from 500 μg to 1000 mg.

In another aspect, the disclosure features a pharmaceutical composition that includes the population of polyribonucleotides of any of the above embodiments (e.g., that is produced by a method as described herein) and a diluent, carrier, or excipient.

### Definitions

To facilitate the understanding of this disclosure, a number of terms are defined below. Terms defined herein have meanings as commonly understood by a person of ordinary skill in the areas relevant to the disclosure. Terms such as “a,” “an,” and “the” are not intended to refer to only a singular entity but include the general class of which a specific example may be used for illustration. The term “or” is used to mean “and/or” unless explicitly indicated to refer to alternatives only or the alternative are mutually exclusive, although the disclosure supports a definition that refers to only alternatives and “and/or”. The terminology herein is used to describe specific embodiments, but their usage is not to be taken as limiting, except as outlined in the claims.

As used herein, any values provided in a range of values include both the upper and lower bounds, and any values contained within the upper and lower bounds.

As used herein, the term “about” refers to a value that is within ±10% of a recited value.

As used herein, the term “adjuvant” refers to a composition (e.g., a compound, polypeptide, nucleic acid, or lipid) that increases an immune response, for example, increases a specific immune response against an immunogen. Increasing an immune response includes intensification or broadening the specificity of either or both antibody and cellular immune responses.

As used herein, the term “carrier” is a compound, composition, reagent, or molecule that facilitates the transport or delivery of a composition (e.g., a circular polyribonucleotide) into a cell by a covalent modification of the circular polyribonucleotide, via a partially or completely encapsulating agent, or a combination thereof. Non-limiting examples of carriers include carbohydrate carriers (e.g., an anhydride-modified phyto glycogen or glycogen-type material), nanoparticles (e.g., a nanoparticle that encapsulates or is covalently linked binds to the circular polyribonucleotide), liposomes, fusosomes, ex vivo differentiated reticulocytes, exosomes, protein carriers (e.g., a protein covalently linked to the circular polyribonucleotide), or cationic carriers (e.g., a cationic lipopolymer or transfection reagent).

As used herein, the terms “circular polyribonucleotide,” “circular RNA,” and “circRNA” are used interchangeably and mean a polyribonucleotide molecule that has a structure having no free ends (i.e., no free 3′ or 5′ ends), for example a polyribonucleotide molecule that forms a circular or end-less structure through covalent or non-covalent bonds. The circular polyribonucleotide may be, e.g., a covalently closed polyribonucleotide.

The term “diluent” means a vehicle including an inactive solvent in which a composition described herein (e.g., a composition including a circular polyribonucleotide) may be diluted or dissolved. A diluent can be an RNA solubilizing agent, a buffer, an isotonic agent, or a mixture thereof. A diluent can be a liquid diluent or a solid diluent. Non-limiting examples of liquid diluents include water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and 1,3-butanediol. Non-limiting examples of solid diluents include calcium carbonate, sodium carbonate, calcium phosphate, dicalcium phosphate, calcium sulfate, calcium hydrogen phosphate, sodium phosphate lactose, sucrose, cellulose, microcrystalline cellulose, kaolin, mannitol, sorbitol, inositol, sodium chloride, dry starch, cornstarch, or powdered sugar.

As used herein, the terms “disease,” “disorder,” and “condition” each refer to a state of sub-optimal health, for example, a state that is or would typically be diagnosed or treated by a medical professional.

As used herein, the term “expression sequence” is a nucleic acid sequence that encodes a product, e.g., a peptide or polypeptide. An exemplary expression sequence that codes for a peptide or polypeptide can include a plurality of nucleotide triads, each of which can code for an amino acid and is termed as a “codon”.

As used herein, the term “GC content” refers to the percentage of guanine (G) and cytosine (C) in a nucleic acid sequence. The formula for calculation of the GC content is (G+C)/(A+G+C+U)×100% (for RNA) or (G+C)/(A+G+C+T)×100% (for DNA). Likewise, the term “uridine content” refers to the percentage of uridine (U) in a nucleic acid sequence. The formula for calculation of the uridine content is U/(A+G+C+U)×100%. Likewise, the term “thymidine content” refers to the percentage of thymidine (T) in a nucleic acid sequence. The formula for calculation of the thymidine content is T/(A+G+C+T)×100%.

By “heterologous” is meant to occur in a context other than in the naturally occurring (native) context. A “heterologous” polynucleotide sequence indicates that the polynucleotide sequence is being used in a way other than what is found in that sequence's native genome. For example, a “heterologous promoter” is used to drive transcription of a sequence that is not one that is natively transcribed by that promoter; thus, a “heterologous promoter” sequence is often included in an expression construct by means of recombinant nucleic acid techniques. The term “heterologous” is also used to refer to a given sequence that is placed in a non-naturally occurring relationship to another sequence; for example, a heterologous coding or non-coding nucleotide sequence is commonly inserted into a genome by genomic transformation techniques, resulting in a genetically modified or recombinant genome.

As used herein, the term “intron fragment” refers to a portion of an intron, where a first intron fragment and a second intron fragment together form an intron, such as a catalytic intron. An intron fragment may be a 5′ portion of an intron (e.g., a 5′ portion of a catalytic intron) or a 3′ portion of an intron (e.g., a 3′ portion of a catalytic intron), such that the 5′ intron fragment and the 3′ intron fragment, together, form a functional intron, such as a functional intron capable of catalytic self-splicing. The term intron fragment is meant to refer to an intron split into two portions. The term intron fragment is not meant to state, imply, or suggest that the two portion or halves are equal in length. The term intron fragment is used synonymously with the term split-intron and may be used instead of the term “half-intron.”

As used herein, the term “impurity” is an undesired substance present in a composition, e.g., a pharmaceutical composition as described herein. In some embodiments, an impurity is a process-related impurity. In some embodiments, an impurity is a product-related substance other than the desired product in the final composition, e.g., other than the active drug ingredient, e.g., circular polyribonucleotide, as described herein. As used herein, the term “process-related impurity” is a substance used, present, or generated in the manufacturing of a composition, preparation, or product that is undesired in the final composition, preparation, or product other than the linear polyribonucleotides described herein. In some embodiments, the process-related impurity is an enzyme used in the synthesis or circularization of polyribonucleotides. As used herein, the term “product-related substance” is a substance or byproduct produced during the synthesis of a composition, preparation, or product, or any intermediate thereof. In some embodiments, the product-related substance is deoxyribonucleotide fragments. In some embodiments, the product-related substance is deoxyribonucleotide monomers. In some embodiments, the product-related substance is one or more of: derivatives or fragments of polyribonucleotides described herein, e.g., fragments of 10, 9, 8, 7, 6, 5, or 4 ribonucleic acids, monoribonucleic acids, diribonucleic acids, or triribonucleic acids.

As used herein, “increasing fitness” or “promoting fitness” of a subject refers to any favorable alteration in physiology, or of any activity carried out by a subject organism, as a consequence of administration of a peptide or polypeptide described herein, including, but not limited to, any one or more of the following desired effects: (1) increased tolerance of biotic or abiotic stress; (2) increased yield or biomass; (3) modified flowering time; (4) increased resistance to pests or pathogens; (4) increased resistance to herbicides; (5) increasing a population of a subject organism (e.g., an agriculturally important insect); (6) increasing the reproductive rate of a subject organism (e.g., insect, e.g., bee or silkworm); (7) increasing the mobility of a subject organism (e.g., insect, e.g. bee or silkworm); (8) increasing the body weight of a subject organism (e.g., insect, e.g., bee or silkworm); (9) increasing the metabolic rate or activity of a subject organism (e.g., insect, e.g., bee or silkworm); (10) increasing pollination (e.g., number of plants pollinated); (11) increasing production of subject organism (e.g., insect, e.g., bee or silkworm) byproducts (e.g., honey from honeybee or silk from silkworm); (12) increasing nutrient content of the subject organism (e.g., insect) (e.g., protein, fatty acids, or amino acids); (13) increasing a subject organism's resistance to pesticides (e.g., a neonicotinoid (e.g., imidacloprid) or an organophosphorus insecticide (e.g., a phosphorothioate, e.g., fenitrothion); or (14) increasing health or reducing disease of a subject organism such as a human or non-human animal. An increase in host fitness can be determined in comparison to a subject organism to which the modulating agent has not been administered.

Conversely, “decreasing fitness” of a subject refers to any unfavorable alteration in physiology, or of any activity carried out by a subject organism, as a consequence of administration of a peptide or polypeptide described herein, including, but not limited to, any one or more of the following intended effects: (1) decreased tolerance of biotic or abiotic stress; (2) decreased yield or biomass; (3) modified flowering time; (4) decreased resistance to pests or pathogens, (4) decreased resistance to herbicides; (5) decreasing a population of a subject organism (e.g., an agriculturally important insect); (6) decreasing the reproductive rate of a subject organism (e.g., insect, e.g., bee or silkworm); (7) decreasing the mobility of a subject organism (e.g., insect, e.g., bee or silkworm); (8) decreasing the body weight of a subject organism (e.g., insect, e.g., bee or silkworm); (9) decreasing the metabolic rate or activity of a subject organism (e.g., insect, e.g., bee or silkworm); (10) decreasing pollination (e.g., number of plants pollinated in a given amount of time) by a subject organism (e.g., insect, e.g., bee or silkworm); (11) decreasing production of subject organism (e.g., insect, e.g., bee or silkworm) byproducts (e.g., honey from a honeybee or silk from a silkworm); (12) decreasing nutrient content of the subject organism (e.g., insect) (e.g., protein, fatty acids, or amino acids); (13) decreasing a subject organism's resistance to pesticides (e.g., a neonicotinoid (e.g., imidacloprid) or an organophosphorus insecticide (e.g., a phosphorothioate, e.g., fenitrothion)); or (14) decreasing health or reducing disease of a subject organism such as a human or non-human animal. A decrease in host fitness can be determined in comparison to a subject organism to which the modulating agent has not been administered. It will be apparent to one of skill in the art that certain changes in the physiology, phenotype, or activity of a subject, e.g., modification of flowering time in a plant, can be considered to increase fitness of the subject or to decrease fitness of the subject, depending on the context (e.g., to adapt to a change in climate or other environmental conditions). For example, a delay in flowering time (e.g., about 10%, 20%, 30%, 40%, 50%, 60%, 70%, 80%, 90%, 95%, 99%, 100% fewer plants in a population flowering at a given calendar date) can be a beneficial adaptation to later or cooler Spring times and thus be considered to increase a plant's fitness; conversely, the same delay in flowering time in the context of earlier or warmer Spring times can be considered to decrease a plant's fitness.

As used interchangeably herein, the terms “linear counterpart” or “linear precursor” refer to a polyribonucleotide molecule (and its fragments) having the same or similar nucleotide sequence (e.g., 100%, 95%, 90%, 85%, 80%, 75%, or any percentage therebetween sequence identity) as a circular polyribonucleotide and having two free ends (i.e., the uncircularized version (and its fragments) of the circularized polyribonucleotide). In some embodiments, the linear counterpart (e.g., a pre-circularized version) is a polyribonucleotide molecule (and its fragments) having the same or similar nucleotide sequence (e.g., 100%, 95%, 90%, 85%, 80%, 75%, or any percentage therebetween sequence identity) and same or similar nucleic acid modifications as a circular polyribonucleotide and having two free ends (i.e., the uncircularized version (and its fragments) of the circularized polyribonucleotide). In some embodiments, the linear counterpart is a polyribonucleotide molecule (and its fragments) having the same or similar nucleotide sequence (e.g., 100%, 95%, 90%, 85%, 80%, 75%, or any percentage therebetween sequence identity) and different or no nucleic acid modifications as a circular polyribonucleotide and having two free ends (i.e., the uncircularized version (and its fragments) of the circularized polyribonucleotide). In some embodiments, a fragment of the polyribonucleotide molecule that is the linear counterpart is any portion of linear counterpart polyribonucleotide molecule that is shorter than the linear counterpart polyribonucleotide molecule. In some embodiments, the linear counterpart further includes a 5′ cap. In some embodiments, the linear counterpart further includes a poly adenosine tail. In some embodiments, the linear counterpart further includes a 3′ UTR. In some embodiments, the linear counterpart further includes a 5′ UTR.

As used herein, the terms “linear RNA,” “linear polyribonucleotide,” and “linear polyribonucleotide molecule” are used interchangeably and mean polyribonucleotide molecule having a 5′ and 3′ end. One or both of the 5′ and 3′ ends may be free ends or joined to another moiety. Linear RNA includes RNA that has not undergone circularization (e.g., is pre-circularized) and can be used as a starting material for circularization through, for example, splint ligation, or chemical, enzymatic, ribozyme- or splicing-catalyzed circularization methods.

As used herein, the term “modified oligonucleotide” means an oligonucleotide containing a nucleotide with at least one modification to the sugar, nucleobase, or internucleotide linkage.

As used herein, the term “modified ribonucleotide” means a ribonucleotide containing a nucleoside with at least one modification to the sugar, nucleobase, or internucleoside linkage.

As used herein, the term “naked delivery” is a formulation for delivery to a cell without the aid of a carrier and without covalent modification to a moiety that aids in delivery to a cell. A naked delivery formulation is free from any transfection reagents, cationic carriers, carbohydrate carriers, nanoparticle carriers, or protein carriers. For example, naked delivery formulation of a circular polyribonucleotide is a formulation that includes a circular polyribonucleotide without covalent modification and is free from a carrier.

As used herein, the terms “nicked RNA,” “nicked linear polyribonucleotide,” and “nicked linear polyribonucleotide molecule” are used interchangeably and mean a polyribonucleotide molecule having a 5′ and 3′ end that results from nicking or degradation of a circular RNA. A “nicked circular RNA” means a circular RNA that has been nicked.

As used herein, the term “non-circular RNA” means total nicked RNA and linear RNA.

The term “optionally substituted X,” as used herein, is intended to be equivalent to “X, wherein X is optionally substituted” (e.g., “alkyl, wherein said alkyl is optionally substituted”). It is not intended to mean that the feature “X” (e.g., alkyl) per se is optional. The term “optionally substituted,” as used herein, refers to having 0, 1, or more substituents (e.g., 0-25, 0-20, 0-10, or 0-5 substituents). For example, a C₁ alkyl group, i.e., methyl, may be substituted with oxo to form a formyl group and further substituted with —OH or —NH₂ to form a carboxyl group or an amido group.

The term “pharmaceutical composition” is intended to also disclose that the circular polyribonucleotide included within a pharmaceutical composition can be used for the treatment of the human or animal body by therapy. It is thus meant to be equivalent to “a circular polyribonucleotide for use in therapy”.

The term “polynucleotide” as used herein means a molecule including one or more nucleic acid subunits, or nucleotides, and can be used interchangeably with “nucleic acid” or “oligonucleotide”. A polynucleotide can include one or more nucleotides selected from adenosine (A), cytosine (C), guanine (G), thymine (T) and uracil (U), or variants thereof. A nucleotide can include a nucleoside and at least 1, 2, 3, 4, 5, 6, 7, 8, 9, 10, or more phosphate (PO₃) groups. A nucleotide can include a nucleobase, a five-carbon sugar (either ribose or deoxyribose), and one or more phosphate groups. Ribonucleotides are nucleotides in which the sugar is ribose. Polyribonucleotides, ribonucleic acids, or RNA, can refer to macromolecules that include multiple ribonucleotides that are polymerized via phosphodiester bonds. Deoxyribonucleotides are nucleotides in which the sugar is deoxyribose.

Polydeoxyribonucleotides, deoxyribonucleic acids, and DNA mean macromolecules that include multiple deoxyribonucleotides that are polymerized via phosphodiester bonds. A nucleotide can be a nucleoside monophosphate or a nucleoside polyphosphate. A nucleotide means a deoxyribonucleoside polyphosphate, such as, e.g., a deoxyribonucleoside triphosphate (dNTP), which can be selected from deoxyadenosine triphosphate (dATP), deoxycytidine triphosphate (dCTP), deoxyguanosine triphosphate (dGTP), uridine triphosphate (dUTP) and deoxythymidine triphosphate (dTTP) dNTPs, which include detectable tags, such as luminescent tags or markers (e.g., fluorophores). A nucleotide can include any subunit that can be incorporated into a growing nucleic acid strand. Such subunit can be an A, C, G, T, or U, or any other subunit that is specific to one or more complementary A, C, G, T or U, or complementary to a purine (i.e., A or G, or variant thereof) or a pyrimidine (i.e., C, T or U, or variant thereof). In some examples, a polynucleotide is deoxyribonucleic acid (DNA), ribonucleic acid (RNA), or derivatives or variants thereof. In some cases, a polynucleotide is a short interfering RNA (siRNA), a microRNA (miRNA), a plasmid DNA (pDNA), a short hairpin RNA (shRNA), small nuclear RNA (snRNA), messenger RNA (mRNA), precursor mRNA (pre-mRNA), antisense RNA (asRNA), to name a few, and encompasses both the nucleotide sequence and any structural embodiments thereof, such as single-stranded, double-stranded, triple-stranded, helical, hairpin, etc. In some cases, a polynucleotide molecule is circular. A polynucleotide can have various lengths. A nucleic acid molecule can have a length of at least about 10 bases, 20 bases, 30 bases, 40 bases, 50 bases, 100 bases, 200 bases, 300 bases, 400 bases, 500 bases, 1 kilobase (kb), 2 kb, 3, kb, 4 kb, 5 kb, 10 kb, 50 kb, or more. A polynucleotide can be isolated from a cell or a tissue. Embodiments of polynucleotides include isolated and purified DNA/RNA molecules, synthetic DNA/RNA molecules, and synthetic DNA/RNA analogs.

Embodiments of polynucleotides, e.g., polyribonucleotides or polydeoxyribonucleotides, include polynucleotides that contain one or more nucleotide variants, including nonstandard nucleotide(s), non-natural nucleotide(s), nucleotide analog(s) or modified nucleotides. Examples of modified nucleotides include, but are not limited to diamino purine, 5-fluorouracil, 5-bromouracil, 5-chlorouracil, 5-iodouracil, hypoxanthine, xanthine, 4-acetylcytosine, 5-(carboxyhydroxylmethyl)uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-D46-isopentenyladenine, uracil-5-oxyacetic acid (v), butoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, 2,6-diaminopurine and the like. In some cases, nucleotides include modifications in their phosphate moieties, including modifications to a triphosphate moiety. Non-limiting examples of such modifications include phosphate chains of greater length (e.g., a phosphate chain having, 4, 5, 6, 7, 8, 9, 10 or more phosphate moieties) and modifications with thiol moieties (e.g., alpha-thiotriphosphate and beta-thiotriphosphates). In some embodiments, nucleic acid molecules are modified at the base moiety (e.g., at one or more atoms that typically are available to form a hydrogen bond with a complementary nucleotide and/or at one or more atoms that are not typically capable of forming a hydrogen bond with a complementary nucleotide), sugar moiety or phosphate backbone. Nucleic acid molecules may also contain amine-modified groups, such as amino allyl 1-dUTP (aa-dUTP) and aminohexyl acrylamide-dCTP (aha-dCTP) to allow covalent attachment of amine reactive moieties, such as N-hydroxy succinimide esters (NHS). Alternatives to standard DNA base pairs or RNA base pairs in the oligonucleotides of the present disclosure can provide higher density in bits per cubic mm, higher safety (resistant to accidental or purposeful synthesis of natural toxins), easier discrimination in photo-programmed polymerases, or lower secondary structure. Such alternative base pairs compatible with natural and mutant polymerases for de novo or amplification synthesis are described in Betz K, Malyshev D A, Lavergne T, Welte W, Diederichs K, Dwyer T J, Ordoukhanian P, Romesberg F E, Marx A. Nat. Chem. Biol. 2012; 8(7):612-4, which is herein incorporated by reference for all purposes.

As used herein, the term “polyribonucleotide cargo” herein includes any sequence including at least one polyribonucleotide. In embodiments, the polyribonucleotide cargo includes one or multiple expression (or coding) sequences, wherein each expression (or coding) sequence encodes a polypeptide. In embodiments, the polyribonucleotide cargo includes one or multiple expression sequences, wherein each expression sequence encodes a polypeptide. In embodiments, the polyribonucleotide cargo includes one or multiple noncoding sequences, such as a polyribonucleotide having regulatory or catalytic functions. In embodiments, the polyribonucleotide cargo includes a combination of expression and noncoding sequences. In embodiments, the polyribonucleotide cargo includes one or more polyribonucleotide sequence described herein, such as one or multiple regulatory elements, internal ribosomal entry site (IRES) elements, or spacer sequences.

As used interchangeably herein, the terms “polyA” and “polyA sequence” refer to an untranslated, contiguous region of a nucleic acid molecule of at least 5 nucleotides in length and consisting of adenosine residues. In some embodiments, a polyA sequence is at least 10, at least 15, at least 20, at least 30, at least 40, or at least 50 nucleotides in length. In some embodiments, a polyA sequence is located 3′ to (e.g., downstream of) an open reason frame (e.g., an open reading frame encoding a polypeptide), and the polyA sequence is 3′ to a termination element (e.g., a Stop codon) such that the polyA is not translated. In some embodiments, a polyA sequence is located 3′ to a termination element and a 3′ untranslated region.

As used herein, the elements of a nucleic acid are “operably connected” or “operably linked” if they are positioned in the vector such that they can be transcribed to form a linear polyribonucleotide that can then be circularized into a circular polyribonucleotide using the methods provided herein.

As used herein, “polypeptide” means a polymer of amino acid residues (natural or unnatural) linked together most often by peptide bonds. The term, as used herein, refers to proteins, polypeptides, and peptides of any size, structure, or function. Polypeptides can include gene products, naturally occurring polypeptides, synthetic polypeptides, homologs, orthologs, paralogs, fragments and other equivalents, variants, and analogs of the foregoing. A polypeptide can be a single molecule or a multi-molecular complex such as a dimer, trimer, or tetramer. They can also include single chain or multichain polypeptides such as antibodies or insulin and can be associated or linked. Most commonly disulfide linkages are found in multichain polypeptides. The term polypeptide can also apply to amino acid polymers in which one or more amino acid residues are an artificial chemical analogue of a corresponding naturally occurring amino acid.

As used herein, the term “plant-modifying polypeptide” refers to a polypeptide that can alter the genetic properties (e.g., increase gene expression, decrease gene expression, or otherwise alter the nucleotide sequence of DNA or RNA), epigenetic properties, or biochemical or physiological properties of a plant in a manner that results in a change in the plant's physiology or phenotype, e.g., an increase or a decrease in plant fitness.

As used herein, the terms “purify,” “purifying,” and “purification” refer to one or more steps or processes of removing impurities (e.g., a process-related impurity (e.g., an enzyme), a process-related substance (e.g., a deoxyribonucleotide fragment, a deoxyribonucleotide monomer)) or by-products (e.g., linear RNA) from a sample containing a mixture circular RNA and linear RNA, among other substances, to produce a composition containing an enriched population of circular RNA with a reduced level of an impurity (e.g., a process-related impurity (e.g., an enzyme), a process-related substance (e.g., deoxyribonucleotide fragment, deoxyribonucleotide monomer)) or by-product (e.g., linear RNA) as compared to the original mixture or in which the linear RNA or substances have been reduced by 40% or more by mass (e.g., 50%, 60%, 70%, 80%, 85%, 90%, 95%, 97%, or 99% or more) relative to a starting mixture.

As used herein, the terms “pure” and “purity” refer to the extent to which an analyte (e.g., circular RNA) has been isolated and is free of other components. In the context of nucleic acids (e.g., polyribonucleotides), purity of an isolated nucleic acid (e.g., circular RNA) can be expressed with regard to the population of nucleic acids that is free of any contaminants, impurities, or by-products (e.g., linear RNA and other substances). For example, purity of a population of circular RNA indicates how much of the population is circular RNA by total mass of the isolated material, which may be determined using, e.g., pure circular RNA as a reference. A level of purity found in the disclosure can be 5%, 10%, 15%, 20%, 25%, 30%, 35%, 40%, 45%, 50%, 55%, 60%, 65%, 70%, 75%, 80%, 85%, 90%, 95%, greater than 95%, or greater than 99% (w/w). In some embodiments, the level of contaminants or impurities or by-products is no more than about 20%, 15%, 12%, 10%, 9%, 8%, 7%, 6%, 5%, 4%, 3%, 2%, or 1% (w/w). Purity can be determined by detecting a level of a specific analyte (e.g., circular RNA) or a specific impurity or by-product (e.g., linear RNA) using gel electrophoresis, spectrophotometry (e.g., NanoDrop by ThermoFisher Scientific), or other technique suitable for measuring purity of a population of nucleic acids and calculating a percentage of the analyte (w/w) relative to the total nucleic acid content (e.g., as determined by an assay known in the art).

As used herein, the phrase “substantially free of one or more impurities or by-products” refers to a property of a sample, such as a sample containing an enriched population of circular RNA, that is free of one or more impurities or by-products (e.g., one or more impurities or by-products disclosed herein) or contains a minimal amount of the one or more impurities or by-products. A minimal amount of the one or more impurities or by-products may be no more than 20% (w/w) (e.g., no more than 19%, 18%, 17%, 16%, 15%, 14%, 13%, 12%, 11%, 10%, 9%, 8%, 7%, 6%, 5%, 4%, 3%, 2%, 1% (w/w), or less). In another example, the sample or the enriched population of circular RNA is substantially free of one or more impurities or by-products if the one or more impurities or by-products are present in an amount that is less than 15% (w/w) (e.g., no more than 14%, 13%, 12%, 11%, 10%, 9%, 8%, 7%, 6%, 5%, 4%, 3%, 2%, 1% (w/w), or less). In another example, the sample or the enriched population of circular RNA is substantially free of one or more impurities or by-products if the one or more impurities or by-products are present in an amount that is less than 10% (w/w) (e.g., no more than 9%, 8%, 7%, 6%, 5%, 4%, 3%, 2%, 1% (w/w), or less). In another example, the sample or the enriched population of circular RNA is substantially free of one or more impurities or by-products if the one or more impurities or by-products are present in an amount that is less than 5% (w/w) (e.g., no more than 4%, 3%, 2%, 1% (w/w) or less). In yet another example, the sample or the enriched population of circular RNA is substantially free of one or more impurities or by-products if the one or more impurities or by-products are present in an amount that is less than 1% (no more than 0.9%, 0.8%, 0.7%, 0.6%, 0.5%, 0.4%, 0.3%, 0.2%, 0.1% (w/w), or less).

As used herein, a “regulatory element” is a moiety, such as a nucleic acid sequence, that modifies expression of an expression sequence within the circular or linear polyribonucleotide.

As used herein, the term “replication element” is a sequence and/or motif useful for replication or that initiates transcription of the circular polyribonucleotide.

As used herein, a “spacer” refers to any contiguous nucleotide sequence (e.g., of one or more nucleotides) that provides distance or flexibility between two adjacent polynucleotide regions.

As used herein, the term “sequence identity” is determined by alignment of two peptide or two nucleotide sequences using a global or local alignment algorithm. Sequences are referred to as “substantially identical” or “essentially similar” when they share at least a certain minimal percentage of sequence identity when optimally aligned (e.g., when aligned by programs such as GAP or BESTFIT using default parameters). GAP uses the Needleman and Wunsch global alignment algorithm to align two sequences over their entire length, maximizing the number of matches and minimizes the number of gaps. Generally, the GAP default parameters are used, with a gap creation penalty=50 (nucleotides)/8 (proteins) and gap extension penalty=3 (nucleotides)/2 (proteins). For nucleotides the default scoring matrix used is nwsgapdna, and for proteins the default scoring matrix is Blosum62 (Henikoff & Henikoff, 1992, PNAS 89, 915-919). Sequence alignments and scores for percentage sequence identity are determined, e.g., using computer programs, such as the GCG Wisconsin Package, Version 10.3, available from Accelrys Inc., 9685 Scranton Road, San Diego, CA 92121-3752 USA, or EmbossWin version 2.10.0 (using the program “needle”). Alternatively, or additionally, percent identity is determined by searching against databases, e.g., using algorithms such as FASTA, BLAST, etc. Sequence identity refers to the sequence identity over the entire length of the sequence.

A “signal sequence” refers to a polypeptide sequence, e.g., between 10 and 45 amino acids in length, which is present at the N-terminus of a polypeptide sequence of a nascent protein which targets the polypeptide sequence to the secretory pathway.

As used herein, a “termination element” is a moiety, such as a nucleic acid sequence, that terminates translation of the expression sequence in the circular or linear polyribonucleotide.

As used herein, the term “total ribonucleotide molecules” means the total amount of any ribonucleotide molecules, including linear polyribonucleotide molecules, circular polyribonucleotide molecules, monomeric ribonucleotides, other polyribonucleotide molecules, fragments thereof, and modified variations thereof, as measured by total mass of the ribonucleotide molecules

As used herein, the term “translation initiation sequence” is a nucleic acid sequence that initiates translation of an expression sequence in the circular polyribonucleotide.

As used herein, the term “yield” refers to the relative amount of an analyte (e.g., a population of circular polyribonucleotides) obtained after a purification step or process as compared to the amount of analyte in the starting material (e.g., a mixed population of polyribonucleotides, such as, e.g., circular and linear polyribonucleotides) (w/w). The yield may be expressed as a percentage. In the context of the disclosure, the amount of analyte (e.g., circular polyribonucleotides) in the starting material and analyte obtained after the purification step can be measured using an assay (e.g., gel electrophoresis or spectrophotometry). The methods of the disclosure can be used to produce a yield of an enriched population of circular polyribonucleotides of about 20% (w/w) or greater relative to the amount present in the starting material, e.g., mixed population of polyribonucleotides. For example, the methods can be used to produce a yield of purified circular polyribonucleotides of about 25%, 30%, 35%, 40%, 45%, 50%, 55%, 65%, 70%, 75%, 80%, 85%, or 90% (w/w) or greater.

Other features and advantages of the invention will be apparent from the following Detailed Description, the drawings, and the claims.

### BRIEF DESCRIPTION OF THE DRAWINGS

FIG. 1 is a schematic drawing showing a method as described herein. On the left is a linear polyribonucleotide. The linear polyribonucleotide is circularized. The target region is attached to the linear polyribonucleotides that are not circularized. An oligonucleotide conjugated to a particle is added to the mixture. The oligonucleotide hybridizes to the target on the linear polyribonucleotides while the circular polyribonucleotides are not bound by the oligonucleotide, thereby separating the linear polyribonucleotides containing the target region from the circular polyribonucleotides that lack the target region. A further target region may optionally be present at the 5′ end of the linear precursor, as shown.

FIG. 2 is a schematic drawing showing a method as described herein. On the left is a linear polyribonucleotide. The linear polyribonucleotide is circularized. The linear polyribonucleotides that are not circularized are polyadenylated, thereby producing a 3′ polyA tail. A polyDT oligonucleotide conjugated to a particle is added to the mixture. The oligo hybridizes to the polyA target on the linear polyribonucleotides while the circular polyribonucleotides are not bound by the oligo, thereby separating the linear polyribonucleotides containing the polyA tail from the circular polyribonucleotides that lack the polyA tail. A further polyA region may optionally be present at the 5′ end of the linear precursor, as shown.

FIG. 3A shows a chromatogram of the in vitro transcription (IVT)-generated sample from Example 1. The chromatogram is annotated with the flow through (FT), elution peak 1 (E1) and elution peak 2 (E2), which are characterized by SDS PAGE as described in Example 1 and as shown in FIG. 3B.

FIG. 3B is a gel showing an SDS PAGE analysis (6% TBE gel) of the IVT-generated mixture of circular and linear RNA in Example 1 following purification and polyadenylation. The first lane is the sample mixture of IVT-generated circRNA and linRNA that was initially loaded (L) onto the column; the second lane is an analysis of the column's flow-through (FT); the third and fourth lanes are an analysis of elution peak E1 (including fractions B7 and B8); and the fifth and sixth lanes are an analysis of peak E2 (including fractions C4 and C5). The fractions analyzed correspond the fractions (FT, E1, and E2) collected from the column chromatography shown in FIG. 3A. The percent purity calculated from the PAGE analysis is shown.

FIG. 4 are gels showing linear byproducts in an IVT mixture in which circular RNA was generated by self-splicing. The gels show the desired circular RNA product, unspliced linear RNA, partly spliced linear RNA, nicked circular RNA, and spliced introns.

FIG. 5 is a gel showing an SDS-PAGE analysis (6% TBE gel) of the IVT-generated mixture of circular RNA and linear RNA byproducts from the oligo(dT) magnetic bead purification runs (Run A and Run B) in Example 2. The second and fifth lanes are the sample mixture of IVT-generated circRNA and linRNA byproducts that was initially incubated with the oligo(dT) magnetic beads (L) (Run A and Run B, respectively); the third and sixth lanes are an analysis of the oligo flow-through (FT) (Run A and Run B, respectively); and the fourth and seventh lanes are an analysis of the elution (Run A and Run B, respectively).

FIG. 6A shows a chromatogram of the IVT-generated sample from Example 2. The chromatogram is annotated with the flowthrough (FT), breakthrough (BT), elution peak 1 (E1) and elution peak 2 (E2), which are characterized by SDS PAGE as described in Example 2 and as shown in FIG. 6B.

FIG. 6B is a gel showing an SDS-PAGE analysis (6% TBE gel) of the IVT-generated mixture of circular RNA and linear RNA byproducts from the oligo(dT) column purification run in Example 2. The second lane is the sample mixture of IVT-generated circRNA and linRNA byproducts that was initially loaded (L) onto the column; the third lane is an analysis of the column's flowthrough (FT); the fourth lane is an analysis of the breakthrough (BT); the fifth lane is an analysis of elution peak E1; and the sixth lane is an analysis of peak E2. The fractions analyzed correspond to the fractions (FT, BT E1, and E2) collected from the column chromatography shown in FIG. 6A.

### DETAILED DESCRIPTION

The present disclosure describes compositions and methods for processing, e.g., purifying, polyribonucleotides. Polyribonucleotides, such as linear or circular polyribonucleotides may be used for a variety of engineering or therapeutic purposes. However, when polyribonucleotides are generated via certain biological reactions, various impurities, byproducts, or incomplete products may be present. The present invention features methods useful to reduce or remove these impurities, byproducts, or incomplete products from a sample in order to produce compositions with a desired polyribonucleotide composition, amount, and/or purity, or a population containing a plurality of polyribonucleotides with a desired polyribonucleotide composition, amount, and/or purity.

In certain embodiments, the methods are useful for purifying a polyribonucleotide that has undergone a splicing reaction. In such an embodiment, the methods may be used to separate spliced polyribonucleotides from non-spliced polyribonucleotides or non-spliced polyribonucleotides from spliced polyribonucleotides. In some embodiments, the methods may be used to separate circular polyribonucleotides (e.g., that have been spliced) from linear polyribonucleotides or linear polyribonucleotides from circular polyribonucleotides. Such purified compositions containing a desired polyribonucleotide may be useful for various downstream applications, such as delivering a polynucleotide cargo (e.g., encoding a gene or protein) to a target cell. The compositions and methods are described in more detail below.

### Methods

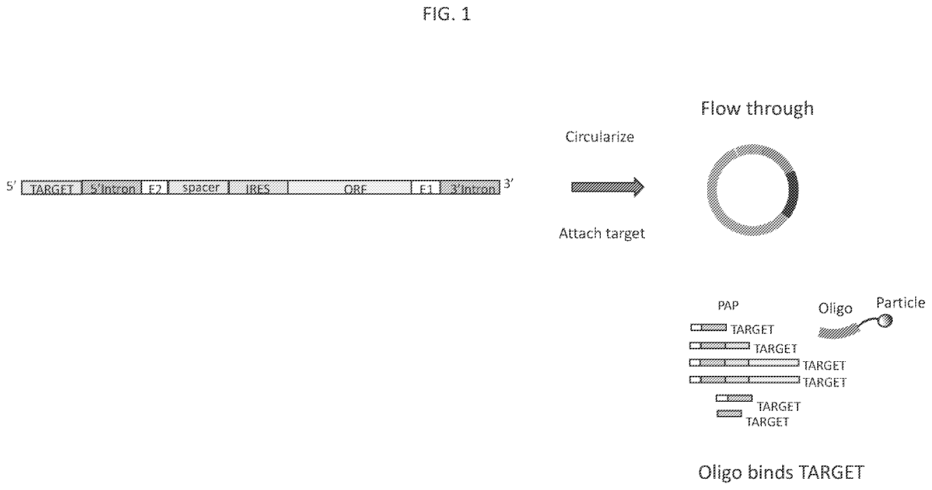

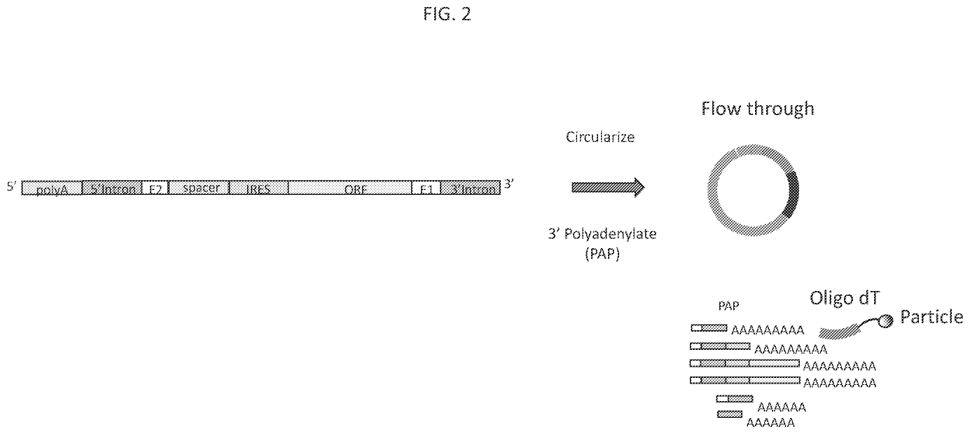

The methods described herein include separating a polyribonucleotide having a target region from a plurality of polyribonucleotides. The method includes providing a sample that includes the plurality of polyribonucleotides. The plurality of polyribonucleotides includes a mixture of linear polyribonucleotides and circular polyribonucleotides. The circular polyribonucleotides may include an open reading frame (ORF) encoding a polypeptide. A subset of the plurality of polyribonucleotides are linear polyribonucleotides. The method further includes attaching the target region to the linear polyribonucleotide. The method also includes contacting the sample with an oligonucleotide that hybridizes to the target region and separating the linear polyribonucleotide having the target region that is hybridized to the oligonucleotide from the plurality of polyribonucleotides in the sample (FIG. 1). In some embodiments, the attachment includes polyadenylation, and the oligonucleotide includes a polyT sequence (FIG. 2).

In some embodiments, the methods described herein include separating a linear polyribonucleotide from a plurality of polyribonucleotides. The plurality of polyribonucleotides include a mixture of linear polyribonucleotides and circular polyribonucleotides. The method includes providing a sample with the plurality of polyribonucleotides, wherein a subset of the plurality of polyribonucleotides include the linear polyribonucleotide; attaching a target region to the linear polyribonucleotide; and contacting the sample with a column that includes a resin with a plurality of particles conjugated to an oligonucleotide that hybridizes to the target region. The method further includes collecting an eluate that includes a portion of the sample that is not hybridized to the oligonucleotide from the plurality of polyribonucleotides in the sample.

In some embodiments, the methods described herein include separating a linear polyribonucleotide from a plurality of polyribonucleotides. The plurality of polyribonucleotides include a mixture of linear polyribonucleotides and circular polyribonucleotides. The method includes circularizing a linear precursor to form the circular polyribonucleotide; providing a sample that includes the plurality of polyribonucleotides, wherein a subset of the plurality of polyribonucleotides include the linear polyribonucleotide; attaching a target region to the linear polyribonucleotide; and contacting the sample with an oligonucleotide that hybridizes to the target region. The method further includes separating the linear polyribonucleotide with the target region that is hybridized to the oligonucleotide from the plurality of polyribonucleotides in the sample.

In some embodiments of any of the methods described herein, the target region is located at a 5′ or 3′ terminus of the polyribonucleotide (e.g., linear or circular polyribonucleotide) and the target region does not contain a polyA sequence. In some embodiments, the target region is located at a 3′ terminus of the polyribonucleotide and does not contain a polyA sequence.

In some embodiments, the oligonucleotide is conjugated (e.g., directly or indirectly) to a particle. The particle may be, for example, a magnetic bead. In some embodiments, the oligonucleotide is conjugated to a resin that includes a plurality of the particles. The resin may include, for example, cross-linked poly[styrene-divinylbenzene], agarose, or SEPHAROSE® agarose. In some embodiments, a column includes the resin.

In some embodiments of any of the methods described herein, separating includes immobilizing the oligonucleotide. The method may include, for example, immobilizing the oligonucleotide, the particle, or a combination thereof.

In some embodiments, the particle is a magnetic particle. The method may include applying a force to the magnetic particle, such as a magnetic force. The particle or bead may be, e.g., a crosslinked agarose, e.g., SEPHAROSE® bead. The method may include applying a force to the bead or particle, such as a mechanical, optical, centrifugal, or acoustic force.

As described herein, the methods may be used to separate, e.g., spliced from non-spliced polyribonucleotides. In some embodiments, the methods described herein include separating a spliced polyribonucleotide from a non-spliced or partially spliced polyribonucleotide. In some embodiments, the spliced polyribonucleotide is a circular polyribonucleotide. In some embodiments, the spliced polyribonucleotide is a linear polyribonucleotide. In some embodiments, the spliced polyribonucleotide lacks an intron or portion thereof, e.g., following a splicing (e.g., self-splicing) event during generation. In some embodiments, the polyribonucleotide having the intron or portion thereof is a linear polyribonucleotide.

In some embodiments, the method further includes washing the bound polyribonucleotide having the target region one or more (e.g., two, three, four five, or more) times. The washing may occur after the contacting and/or after separating step.

In some embodiments, the method further includes performing a first elution step to release the bound polyribonucleotide with the target region from the polyribonucleotide having the target region. The first elution step may include adding a first buffer and/or heating the sample, e.g., to at least 50° C., 55° C., 60° C., 65° C., 70° C., 75° C., 80° C., or higher.

In some embodiments, the method further includes performing a second elution step. The second elution step may include adding a second buffer and/or heating the sample, e.g., to at least 50° C., 55° C., 60° C., 65° C., 70° C., 75° C., 80° C., or higher. In some embodiments, the second buffer includes a denaturing agent, e.g., formamide or urea. The second buffer may include, e.g., from about 40% to about 60% formamide (e.g., about 40%, 45%, 50%, 55%, or 60% formamide).

In some embodiments, the method includes incubating the sample with the oligonucleotide for at least ten (e.g., at least 20, 30, 40, 50, 60, 70, 80, 90, 100, 110, 120, or more) minutes.

In some embodiments, the method includes collecting a portion of the sample that is not bound by the oligonucleotide.

In some embodiments, the method includes providing a plurality of oligonucleotides, wherein each oligonucleotide hybridizes to a distinct target region. Each oligonucleotide may be, e.g., conjugated to a particle, e.g., a magnetic particle or a bead.

In some embodiments, the method includes providing the oligonucleotide at a molar ratio of 10:1 to 1:10 (e.g., 10:1, 5:1, 2:1, 1:2, 1:5, or 1:10) to the polyribonucleotide, e.g., polyribonucleotide containing the target region.

In some embodiments, the method includes providing a sample of particles, e.g., beads, e.g., magnetic beads. The particles may be present in a vessel, e.g., a microcentrifuge tube, or packed in a column. The particles may be conjugated to the oligonucleotide. The method may include flowing the mixture of polyribonucleotides over the column containing the particles. As such, the polyribonucleotides bound by the oligonucleotide will bind the column. In some embodiments, the particles are conjugated directly to an oligonucleotide, e.g., configured to hybridize to the target region of the polyribonucleotide.

In some embodiments, e.g., when using a magnetic particle, the method may include pelleting the magnetic particles, e.g., in a vessel (e.g., microcentrifuge tube) by providing a permanent magnet.

In some embodiments, the methods described herein enrich an amount of the desired polyribonucleotide in the sample. For example, the method may enrich the amount of the desired (e.g., spliced, e.g., circular) polyribonucleotide by at least 10%, (e.g., at least 20%, 30%, 40%, 50%, 55%, 60%, 65%, 70%, 75%, 80%, 85%, 90%, 95%, 97%, 99%, or more) relative to the sample prior to purification.

In some embodiments, the methods of purification result in a circular polyribonucleotide that has less than 50% (mol/mol) (e.g., less than 40%, 30%, 20%, 10%, 5%, 4%, 3%, 2%, or 1% (mol/mol)) linear polyribonucleotides.

In some embodiments, the methods described herein separate at least 500 μg (e.g., at least 600 μg, 700 μg, 800 μg, 900 μg, 1 mg, 2 mg, 3 mg, 4 mg, 5 mg, 6 mg, 7 mg, 8 mg, 9 mg, 10 mg, 20 mg, 30 mg, 40 mg, 50 mg, 60 mg, 70 mg, 80 mg, 90 mg, 100 mg, 200 mg, 300 mg, 400 mg, 500 mg, 600 mg, 700 mg, 800 mg, 900 mg, 1,000 mg, or more) of the linear polyribonucleotide with the target region. In some embodiments, the method separates from 500 μg to 1,000 mg of the linear polyribonucleotide including the target region.

### Methods of Attachment

The methods described herein include attaching a target region to a linear polynucleotide. For example, the method may include attached a target region to a 3′ or 5′ end of a linear polyribonucleotide. In some embodiments, the method includes attaching a region to a 3′ or 5′ end of a linear polyribonucleotide, and the target region is not located at the 3′ or 5′ terminus of the linear polyribonucleotide. For example, a polyribonucleotide containing the target region may be attached to the terminus and the target region is ligated to the terminus of the linear polyribonucleotide while a flanking region forms a new 5′ or 3′ terminus of the linear polyribonucleotide, e.g., after attachment. Attachment may be performed by ligating the target region or portion thereof to the linear polyribonucleotide. In other embodiments, attachment may be performed by polyadenylation, where one or more adenosine ribonucleotides are added to the terminus (e.g., 3′ terminus) of the linear polyribonucleotide, e.g., to produce a polyA tail. Such methods include providing a polyA polymerase, which attaches adenosine monophosphate units from adenosine triphosphate to the RNA while cleaving off pyrophosphate. In some embodiments, a portion of the target region is attached during an attachment step. For example, in some embodiments, the linear polyribonucleotide contains a portion of the target region, and the attachment step includes attaching the remaining part of the target region.

The target region or a polyribonucleotide containing a target region may be attached according to any available technique, including, but not limited to chemical methods and enzymatic methods.

Such enzymatic methods include, for example, providing a ligase (e.g., RNA ligase), which attaches free ends of linear RNA, e.g., a 3′ end of the linear polyribonucleotide and a 5′ end of the target region or a 5′ end of the linear polyribonucleotide and a 3′ end of the target region.

In one example, either the 5′ or 3′ end of the linear polyribonucleotide can encode a ligase ribozyme sequence such that during in vitro transcription, the resultant linear polyribonucleotide includes an active ribozyme sequence capable of ligating the 5′ end of the linear polyribonucleotide or the 3′ end of the linear polyribonucleotide to the target region. The ligase ribozyme may be derived from the Group I Intron, Hepatitis Delta Virus, Hairpin ribozyme or may be selected by SELEX (systematic evolution of ligands by exponential enrichment).

In another example, a target region may be attached to the linear polyribonucleotide using at least one non-nucleic acid moiety. For example, the at least one non-nucleic acid moiety may react with regions or features near the 5′ terminus or near the 3′ terminus of the linear polyribonucleotide in order to attach to the linear polyribonucleotide. In another example, the at least one non-nucleic acid moiety may be located in or linked to or near the 5′ terminus or the 3′ terminus of the linear polyribonucleotide. The non-nucleic acid moieties may be homologous or heterologous. As a non-limiting example, the non-nucleic acid moiety may be a linkage such as a hydrophobic linkage, ionic linkage, a biodegradable linkage or a cleavable linkage. As another non-limiting example, the non-nucleic acid moiety is a ligation moiety. As yet another non-limiting example, the non-nucleic acid moiety may be an oligonucleotide or a peptide moiety, such as an aptamer or a non-nucleic acid linker as described herein.

In another example, the linear polyribonucleotide may be spliced to the target region. In some embodiments, the linear polyribonucleotide and the target region together may include loop E sequence to ligate. In another embodiment, the linear polyribonucleotide and the target region may include a circularizing intron, e.g., a 5′ and 3′ slice junction, or a circularizing catalytic intron such as a Group I, Group II or Group III Introns. Nonlimiting examples of group I intron self-splicing sequences may include self-splicing permuted intron-exon sequences derived from T4 bacteriophage gene td, and the intervening sequence (IVS) rRNA of Tetrahymena.

In another example, a target region may be attached to the linear polyribonucleotide by a non-nucleic acid moiety that causes an attraction between atoms, molecular surfaces at, near, or linked to the 5′ and 3′ ends of the linear polyribonucleotide. The linear polyribonucleotide may be attached to the target region by intermolecular forces or intramolecular forces. Non-limiting examples of intermolecular forces include dipole-dipole forces, dipole-induced dipole forces, induced dipole-induced dipole forces, Van der Waals forces, and London dispersion forces. Non-limiting examples of intramolecular forces include covalent bonds, metallic bonds, ionic bonds, resonant bonds, agnostic bonds, dipolar bonds, conjugation, hyperconjugation and antibonding.

In another example, the linear polyribonucleotide may include a ribozyme RNA sequence near the 5′ terminus and the target region may include a ribozyme RNA sequence near the 3′ terminus, or vice versa. The ribozyme RNA sequence may covalently link to a peptide when the sequence is exposed to the remainder of the ribozyme. The peptides covalently linked to the ribozyme RNA sequence near the 5′ terminus and the 3 ′terminus may associate with each other, thereby attaching the target region to the linear polyribonucleotide. Non-limiting examples of ribozymes for use in the linear primary constructs or linear polyribonucleotides of the present invention or a non-exhaustive listing of methods to incorporate or covalently link peptides are described in US patent application No. US20030082768, the contents of which is here in incorporated by reference in its entirety.

In yet another example, chemical methods of circularization may be used to attach the target region to the linear polyribonucleotide. Such methods may include, but are not limited to click chemistry (e.g., alkyne and azide-based methods, or clickable bases), olefin metathesis, phosphoramidate ligation, hemiaminal-imine crosslinking, base modification, and any combination thereof.

### Methods of Circularization

Circularization may be performed using methods including, e.g., recombinant technology or chemical synthesis. For example, a DNA molecule used to produce an RNA circle can include a DNA sequence of a naturally occurring original nucleic acid sequence, a modified version thereof, or a DNA sequence encoding a synthetic polypeptide not normally found in nature (e.g., chimeric molecules or fusion proteins). DNA and RNA molecules can be modified using a variety of techniques including, but not limited to, classic mutagenesis techniques and recombinant techniques, such as site-directed mutagenesis, chemical treatment of a nucleic acid molecule to induce mutations, restriction enzyme cleavage of a nucleic acid fragment, ligation of nucleic acid fragments, polymerase chain reaction (PCR) amplification or mutagenesis of selected regions of a nucleic acid sequence, synthesis of oligonucleotide mixtures and ligation of mixture groups to “build” a mixture of nucleic acid molecules and combinations thereof.

In some embodiments, a linear polyribonucleotide for circularization is cyclized, or concatemerized. In some embodiments, the linear polyribonucleotide for circularization is cyclized in vitro prior to separation, formulation, and/or delivery. In some embodiments, the circular polyribonucleotide is a mixture with linear polyribonucleotides. In some embodiments, the linear polyribonucleotides have the same nucleic acid sequence as the circular polyribonucleotides.

In some embodiments, a linear polyribonucleotide for circularization is cyclized, or concatemerized using a chemical method to form a circular polyribonucleotide. In some chemical methods, the 5′-end and the 3′-end of the nucleic acid (e.g., a linear polyribonucleotide for circularization) includes chemically reactive groups that, when close together, may form a new covalent linkage between the 5′-end and the 3′-end of the molecule. The 5′-end may contain an NHS-ester reactive group and the 3′-end may contain a 3′-amino-terminated nucleotide such that in an organic solvent the 3′-amino-terminated nucleotide on the 3′-end of a linear RNA molecule will undergo a nucleophilic attack on the 5′-NHS-ester moiety forming a new 5′-/3′-amide bond.

In some embodiments, a DNA or RNA ligase is used to enzymatically link a 5′-phosphorylated nucleic acid molecule (e.g., a linear polyribonucleotide for circularization) to the 3′-hydroxyl group of a nucleic acid (e.g., a linear nucleic acid) forming a new phosphorodiester linkage. In an example reaction, a linear polyribonucleotide for circularization is incubated at 37° C. for 1 hour with 1-10 units of T4 RNA ligase (New England Biolabs, Ipswich, MA) according to the manufacturer's protocol. The ligation reaction may occur in the presence of a linear nucleic acid capable of base-pairing with both the 5′- and 3′-region in juxtaposition to assist the enzymatic ligation reaction. In some embodiments, the ligation is splint ligation. For example, a splint ligase, like SplintR® ligase, can be used for splint ligation, RNA ligase II, T4 RNA ligase, or T4 DNA ligase. For splint ligation, a single stranded polynucleotide (splint), like a single stranded RNA, can be designed to hybridize with both termini of a linear polyribonucleotide, so that the two termini can be juxtaposed upon hybridization with the single-stranded splint. Splint ligase can thus catalyze the ligation of the juxtaposed two termini of the linear polyribonucleotide, generating a circular polyribonucleotide.

In some embodiments, a DNA or RNA ligase is used in the synthesis of the circular polynucleotides. In some embodiments, either the 5′- or 3′-end of the linear polyribonucleotide for circularization can encode a ligase ribozyme sequence such that during in vitro transcription, the resultant linear polyribonucleotide for circularization includes an active ribozyme sequence capable of ligating the 5′-end of the linear polyribonucleotide for circularization to the 3′-end of the linear polyribonucleotide for circularization. The ligase ribozyme may be derived from the Group I Intron, Hepatitis Delta Virus, Hairpin ribozyme or may be selected by SELEX (systematic evolution of ligands by exponential enrichment). The ribozyme ligase reaction may take 1 to 24 hours at temperatures between 0 and 37° C.

In some embodiments, a linear polyribonucleotide for circularization is cyclized or concatemerized by using at least one non-nucleic acid moiety. In one embodiment, the at least one non-nucleic acid moiety reacts with regions or features near the 5′ terminus and/or near the 3′ terminus of the linear polyribonucleotide for circularization in order to cyclize or concatemerized the linear polyribonucleotide for circularization. In another embodiment, the at least one non-nucleic acid moiety is located in or linked to or near the 5′ terminus and/or the 3′ terminus of the linear polyribonucleotide for circularization. The non-nucleic acid moieties contemplated may be homologous or heterologous. As a non-limiting example, the non-nucleic acid moiety is a linkage such as a hydrophobic linkage, ionic linkage, a biodegradable linkage, and/or a cleavable linkage. As another non-limiting example, the non-nucleic acid moiety is a ligation moiety. As yet another non-limiting example, the non-nucleic acid moiety is an oligonucleotide or a peptide moiety, such as an aptamer or a non-nucleic acid linker as described herein.

In some embodiments, the linear polyribonucleotide for circularization is synthesized using IVT and an RNA polymerase, where the nucleotide mixture used for IVT may contain an excess of guanosine monophosphate relative to guanosine triphosphate to preferentially produce RNA with a 5′ monophosphate; the purified IVT product may be circularized using a splint DNA.

In some embodiments, a linear polyribonucleotide for circularization is cyclized or concatemerized due to a non-nucleic acid moiety that causes an attraction between atoms, molecular surfaces at, near or linked to the 5′ and 3′ ends of the linear polyribonucleotide for circularization. As a non-limiting example, one or more linear polyribonucleotides for circularization may be cyclized or concatemerized by intermolecular forces or intramolecular forces. Non-limiting examples of intermolecular forces include dipole-dipole forces, dipole-induced dipole forces, induced dipole-induced dipole forces, Van der Waals forces, and London dispersion forces. Non-limiting examples of intramolecular forces include covalent bonds, metallic bonds, ionic bonds, resonant bonds, agnostic bonds, dipolar bonds, conjugation, hyperconjugation and antibonding.

In some embodiments, a linear polyribonucleotide for circularization includes a ribozyme RNA sequence near the 5′ terminus and near the 3′ terminus. The ribozyme RNA sequence may covalently link to a peptide when the sequence is exposed to the remainder of the ribozyme. In one embodiment, the peptides covalently linked to the ribozyme RNA sequence near the 5′ terminus and the 3 ′terminus may associate with each other causing a linear polyribonucleotide for circularization to cyclize or concatemerize. In another embodiment, the peptides covalently linked to the ribozyme RNA near the 5′ terminus and the 3′ terminus may cause the linear primary construct or linear mRNA to cyclize or concatemerize after being subjected to ligated using various methods known in the art such as, but not limited to, protein ligation. Non-limiting examples of ribozymes for use in the linear primary constructs or linear RNA of the present invention or a non-exhaustive listing of methods to incorporate and/or covalently link peptides are described in US patent application No. US20030082768, the contents of which is here in incorporated by reference in its entirety.

In some embodiments, a linear polyribonucleotide for circularization includes a 5′ triphosphate of the nucleic acid converted into a 5′ monophosphate, e.g., by contacting the 5′ triphosphate with RNA 5′ pyrophosphohydrolase (RppH) or an ATP diphosphohydrolase (apyrase). In some embodiments, the 5′ end of at least a portion of the linear polyribonucleotides includes a monophosphate moiety. In some embodiments, the population of polyribonucleotides including circular and linear polyribonucleotides is contacted with RppH prior to digesting at least a portion of the linear polyribonucleotides with a 5′ exonuclease and/or a 3′ exonuclease. Alternately, converting the 5′ triphosphate of the linear polyribonucleotide for circularization into a 5′ monophosphate may occur by a two-step reaction including: (a) contacting the 5′ nucleotide of the linear polyribonucleotide for circularization with a phosphatase (e.g., Antarctic Phosphatase, Shrimp Alkaline Phosphatase, or Calf Intestinal Phosphatase) to remove all three phosphates; and (b) contacting the 5′ nucleotide after step (a) with a kinase (e.g., Polynucleotide Kinase) that adds a single phosphate.

In some embodiments, the linear polyribonucleotide includes an internal splicing element that when replicated the spliced ends are joined together. Some examples include miniature introns (<100 nt) with splice site sequences and short inverted repeats (30-40 nt) such as AluSq2, AluJr, and AluSz, inverted sequences in flanking introns, A/u elements in flanking introns, and motifs found in (suptable4 enriched motifs) cis-sequence elements proximal to back splice events such as sequences in the 200 bp preceding (upstream of) or following (downstream from) a back splice site with flanking exons. In some embodiments, the linear polyribonucleotide includes at least one repetitive nucleotide sequence described elsewhere herein as an internal splicing element. In such embodiments, the repetitive nucleotide sequence includes repeated sequences from the Alu family of introns. In some embodiments, a splicing-related ribosome binding protein can regulate circular polyribonucleotide biogenesis (e.g., the Muscle blind and Quaking (QKI) splicing factors).

In some embodiments, the linear polyribonucleotide includes canonical splice sites that flank head-to-tail junctions of the circular polyribonucleotide.

In some embodiments, the linear polyribonucleotide includes a bulge-helix-bulge motif, including a 4-base pair stem flanked by two 3-nucleotide bulges. Cleavage occurs at a site in the bulge region, generating characteristic fragments with terminal 5′-hydroxyl group and 2′, 3′-cyclic phosphate. Circularization proceeds by nucleophilic attack of the 5′—OH group onto the 2′, 3′-cyclic phosphate of the same molecule forming a 3′, 5′-phosphodiester bridge.

In some embodiments, the linear polyribonucleotide includes a multimeric repeating RNA sequence that harbors a HPR element. The HPR includes a 2′,3′-cyclic phosphate and 5′-OH termini. The HPR element self-processes the 5′- and 3′-ends of the linear polyribonucleotide, thereby ligating the ends together.

In embodiments, linear polyribonucleotides are cyclized or concatenated by self-splicing. In some embodiments, the linear polyribonucleotide includes a sequence that mediates self-ligation. In one embodiment, the linear polyribonucleotide includes a HDV sequence, e.g., HDV replication domain conserved sequence, GGCUCAUCUCGACAAGAGGCGGCAGUCCUCAGUACUCUUACUCUUUUCUGUAAAGAGGAGACUG CUGGACUCGCCGCCCAAGUUCGAGCAUGAGCC (Beeharry et al 2004) (SEQ ID NO: 2) or GGCUAGAGGCGGCAGUCCUCAGUACUCUUACUCUUUUCUGUAAAGAGGAGACUGCUGGACUCGC CGCCCGAGCC (SEQ ID NO: 3), to self-ligate. In one embodiment, the linear polyribonucleotide includes loop E sequence (e.g., in PSTVd) to self-ligate. In another embodiment, the linear polyribonucleotide includes a self-circularizing intron, e.g., a 5′ and 3′ slice junction, or a self-circularizing catalytic intron such as a Group I, Group II or Group III Introns. Nonlimiting examples of group I intron self-splicing sequences include self-splicing permuted intron-exon sequences derived from T4 bacteriophage gene td, and the intervening sequence (IVS) rRNA of Tetrahymena, a cyanobacterium Anabaena pre-tRNA gene, or a Tetrahymena pre-rRNA.

In some embodiments, the linear polyribonucleotide includes catalytic intron fragments, such as a 3′ half of Group I catalytic intron fragment and a 5′ half of Group I catalytic intron fragment. The first and second annealing regions may be positioned within the catalytic intron fragments. Group I catalytic introns are self-splicing ribozymes that catalyze their own excision from mRNA, tRNA, and rRNA precursors via two-metal ion phosphoryl transfer mechanism. Importantly, the RNA itself self-catalyzes the intron removal without the requirement of an exogenous enzyme, such as a ligase.

In some embodiments, the 3′ half of Group I catalytic intron fragment and the 5′ half of Group I catalytic intron fragment are from a cyanobacterium Anabaena pre-tRNA-Leu gene, or a Tetrahymena pre-rRNA.

In some embodiments, the 3′ half of Group I catalytic intron fragment and the 5′ half of Group I catalytic intron fragment are from a Cyanobacterium Anabaena pre-tRNA-Leu gene, and the 3′ exon fragment includes the first annealing region and the 5′ exon fragment includes the second annealing region. The first annealing region may include, e.g., from 5 to 50, e.g., from 10 to 15 (e.g., 10, 11, 12, 13, 14, or 15) ribonucleotides and the second annealing region may include, e.g., from 5 to 50, e.g., from 10 to 15 (e.g., 10, 11, 12, 13, 14, or 15) ribonucleotides.

In some embodiments, the 3′ half of Group I catalytic intron fragment and the 5′ half of Group I catalytic intron fragment are from a Tetrahymena pre-rRNA, and the 3′ half of Group I catalytic intron fragment includes the first annealing region and the 5′ exon fragment includes the second annealing region. In some embodiments, the 3′ exon includes the first annealing region and the 5′ half of Group I catalytic intron fragment includes the second annealing region. The first annealing region may include, e.g., from 6 to 50, e.g., from 10 to 16 (e.g., 10, 11, 12, 13, 14, 15, or 16) ribonucleotides, and the second annealing region may include, e.g., from 6 to 50, e.g., from 10 to 16 (e.g., 10, 11, 12, 13, 14, 15, or 16) ribonucleotides.

In some embodiments, the 3′ half of Group I catalytic intron fragment and the 5′ half of Group I catalytic intron fragment are from a cyanobacterium Anabaena pre-tRNA-Leu gene, a Tetrahymena pre-rRNA, or a T4 phage td gene.

In some embodiments, the 3′ half of Group I catalytic intron fragment and the 5′ Group I catalytic intron fragment are from a T4 phage td gene. The 3′ exon fragment may include the first annealing region and the 5′ half of Group I catalytic intron fragment may include the second annealing region. The first annealing region may include, e.g., from 2 to 16, e.g., 10 to 16 (e.g., 2, 3, 4, 5, 6, 7, 8, 9, 10, 11, 12, 13, 14, 15, or 16) ribonucleotides, and the second annealing region may include, e.g., from 2 to 16, e.g., 10 to 16 (e.g., 2, 3, 4, 5, 6, 7, 8, 9, 10, 11, 12, 13, 14, 15, or 16) ribonucleotides.

In some embodiments, the 3′ half of Group I catalytic intron fragment is the 5′ terminus of the linear polynucleotide.

In some embodiments, the 5′ half of Group I catalytic intron fragment is the 3′ terminus of the linear polyribonucleotide.

In some embodiments, linear polyribonucleotides for circularization include complementary sequences, including either repetitive or nonrepetitive nucleic acid sequences within individual introns or across flanking introns.

In some embodiments, chemical methods of circularization may be used to generate the circular polyribonucleotide. Such methods may include, but are not limited to click chemistry (e.g., alkyne and azide-based methods, or clickable bases), olefin metathesis, phosphoramidate ligation, hemiaminal-imine crosslinking, base modification, and any combination thereof.

In some embodiments, enzymatic methods of circularization may be used to generate the circular polyribonucleotide. In some embodiments, a ligation enzyme, e.g., DNA or RNA ligase, may be used to generate a template of the circular polyribonucleotide or complement, a complementary strand of the circular polyribonucleotide, or the circular polyribonucleotide

In another embodiment, circular polyribonucleotide is produced using a deoxyribonucleotide template transcribed in a cell-free system (e.g., by in vitro transcription) to produce a linear polyribonucleotide. The linear polyribonucleotide produces a splicing-compatible polyribonucleotide, which may be self-spliced to produce a circular polyribonucleotide.

In some embodiments, a circular polyribonucleotide is produced (e.g., in a cell-free system) by providing a linear polyribonucleotide; and self-splicing the linear polyribonucleotide under conditions suitable for splicing of the 3′ and 5′ splice sites of the linear polyribonucleotide, thereby producing a circular polyribonucleotide.

In some embodiments, a circular polyribonucleotide is produced by providing a deoxyribonucleotide encoding a linear polyribonucleotide; transcribing the deoxyribonucleotide in a cell-free system to produce the linear polyribonucleotide; optionally purifying the splicing-compatible linear polyribonucleotide; and self-splicing the linear polyribonucleotide under conditions suitable for splicing of the 3′ and 5′ splice sites of the linear polyribonucleotide, thereby producing a circular polyribonucleotide.

In some embodiments, a circular polyribonucleotide is produced by providing a deoxyribonucleotide encoding a linear polyribonucleotide; transcribing the deoxyribonucleotide in a cell-free system to produce the linear polyribonucleotide, wherein the transcribing occurs in a solution under conditions suitable for splicing of the 3′ and 5′ splice sites of the linear polyribonucleotide, thereby producing a circular polyribonucleotide. In some embodiments, the linear polyribonucleotide comprises a 5′ split-intron and a 3′ split-intron (e.g., a self-splicing construct for producing a circRNA). In some embodiments, the linear polyribonucleotide comprises a 5′ annealing region and a 3′ annealing region.

In some embodiments the linear polyribonucleotide is produced from a deoxyribonucleic acid, e.g., a deoxyribonucleic acid described herein, such as a DNA vector, a linearized DNA vector, or a cDNA. In some embodiments, the linear polyribonucleotide is transcribed from the deoxyribonucleic acid by transcription in a cell-free system (e.g., in vitro transcription).

In some embodiments, a circular polyribonucleotide is produced in a cell, e.g., a prokaryotic cell or a eukaryotic cell. In some embodiments, an exogenous polyribonucleotide is provided to a cell (e.g., a linear polyribonucleotide described herein or a DNA molecule encoding for the transcription of a linear polyribonucleotide described here). The linear polyribonucleotide may be transcribed in the cell from an exogenous DNA molecule provided to the cell. The linear polyribonucleotide may be transcribed in the cell from an exogenous recombinant DNA molecule transiently provided to the cell. In some embodiments, the exogenous DNA molecule does not integrate into the cell's genome. In some embodiments, the linear polyribonucleotide is transcribed in the cell from a recombinant DNA molecule that is incorporated into the cell's genome.

Methods of making circular polyribonucleotides described herein are described in, for example, Khudyakov & Fields, Artificial DNA: Methods and Applications, CRC Press (2002); in Zhao, Synthetic Biology: Tools and Applications, (First Edition), Academic Press (2013); Muller and Appel, from RNA Biol, 2017, 14(8):1018-1027; and Egli & Herdewijn, Chemistry and Biology of Artificial Nucleic Acids, (First Edition), Wiley-VCH (2012). Other methods of making circular polyribonucleotides are described, for example, in International Publication No. WO2022/247943, US Patent No. U.S. Ser. No. 11/000,547, International Publication No. 2018/191722, International Publication No. WO2019/236673, International Publication No. WO2020/023595, International Publication No. WO2022/204460, International Publication No. WO2022/204464, and International Publication No. WO2022/204466.

Various methods of making circular polyribonucleotides are also described elsewhere (see, e.g., U.S. Pat. Nos. 6,210,931, 5,773,244, 5,766,903, U.S. Pat. Nos. 5,712,128, 5,426,180, US Publication No. US20100137407, International Publication No. WO1992001813, International Publication No. WO2010084371, and Petkovic et al., Nucleic Acids Res. 43:2454-65 (2015); the contents of each of which are herein incorporated by reference in their entirety).

### Oligonucleotides

The oligonucleotides described herein are configured to hybridize to a target region of a polyribonucleotide. In some embodiments, the oligonucleotide has at least 80% (e.g., at least 85%, 90%, 95%, 97%, 99%, or 100%) complementarity an equal length portion of the target region. In some embodiments, the oligonucleotide has at most 10, 9, 8, 7, 6, 5, 4, 3, 2, or 1 mismatch to the target region of the polyribonucleotide. In some embodiments, the oligonucleotide has no mismatches to the target region. In some embodiments the oligonucleotide may be a modified oligonucleotide (e.g., having modified phosphate, sugar, or base). In some embodiments, the oligonucleotide contains a portion configured to hybridize to the target region and a portion that does not hybridize to the target region (e.g., a terminal region).

The oligonucleotide may be, for example, at least 5 nucleotides (e.g., at least 6, 7, 8, 9, 10, 11, 12, 13, 14, 15, 16, 17, 18, 19, 20, 21, 22, 23, 24, 25, 26, 27, 28, 29, 30, 35, 40, 45, 50, 55, 60, 65, 70, 75, 80, 85, 90, 95, 100, or more nucleotides) in length. In some embodiments, the oligonucleotide is, e.g., from 5-100, 5-95, 10-90, 10-80, 12-60, 15-50, 15-40, 15-30, 18-30, 20-25, or 20-22 nucleotides in length.

The oligonucleotide may have, for example, a GC content of from 30-70%, e.g., 30%, 35%, 40%, 45%, 50%, 55%, 60%, 65%, or 70%. The oligonucleotide may have a melting temperature (Tm) of, for example, from about 45° C. to about 75° C., e.g., about 46° C., 47° C., 48° C., 49° C., 50° C., 51° C., 52° C., 53° C., 54° C., 55° C., 56° C., 57° C., 58° C., 59° C., 60° C., 61° C., 62° C., 63° C., 64° C., 65° C., 66° C., 67° C., 68° C., 69° C., 70° C., 71° C., 72° C., 73° C., 74° C., or 75° C.

In some embodiments, the oligonucleotide includes a polydT or polyU sequence. Such a sequence may be useful for binding (e.g., a hybridizing to) a polyA or polydA target region. In some embodiments, the oligonucleotide includes at least 10 (e.g., at least 11, 12, 13, 14, 15, 16, 17, 18, 19, 20, 21, 22, 23, 24, 25, 26, 27, 28, 29, 30, or more, e.g., at least 25) consecutive thymidines or uridines. In some embodiments, the oligonucleotide is a poly(dT)₂₅.

### Target Region

The target region of a polyribonucleotide as described herein is configured to hybridize to an oligonucleotide. In some embodiments, the target region has at least 80% (e.g., at least 85%, 90%, 95%, 97%, 99%, or 100%) complementarity an equal length portion of the oligonucleotide. In some embodiments, the target region has at most 10, 9, 8, 7, 6, 5, 4, 3, 2, or 1 mismatch to the oligonucleotide. In some embodiments, the target region has no mismatches to the oligonucleotide. In some embodiments the target region may contain modified nucleotides (e.g., having modified phosphate, sugar, or base). In some embodiments, the target region contains a portion configured to hybridize to the oligonucleotide and a portion that does not hybridize to the oligonucleotide (e.g., a terminal region).

The target region may be, for example, at least 5 nucleotides (e.g., at least 6, 7, 8, 9, 10, 11, 12, 13, 14, 15, 16, 17, 18, 19, 20, 21, 22, 23, 24, 25, 26, 27, 28, 29, 30, 35, 40, 45, 50, 55, 60, 65, 70, 75, 80, 85, 90, 95, 100, or more nucleotides) in length. In some embodiments, the target region is, e.g., from 5-100, 5-95, 10-90, 10-80, 12-60, 15-50, 15-40, 15-30, 18-30, 20-25, or 20-22 nucleotides in length.

The target region may have, for example, a GC content of from 30-70%, e.g., 30%, 35%, 40%, 45%, 50%, 55%, 60%, 65%, or 70%. The target region may have a melting temperature (Tm) of, for example, from about 45° C. to about 75° C., e.g., about 46° C., 47° C., 48° C., 49° C., 50° C., 51° C., 52° C., 53° C., 54° C., 55° C., 56° C., 57° C., 58° C., 59° C., 60° C., 61° C., 62° C., 63° C., 64° C., 65° C., 66° C., 67° C., 68° C., 69° C., 70° C., 71° C., 72° C., 73° C., 74° C., or 75° C.

In some embodiments, the target region includes a polyA or polydA sequence. Such a sequence may be useful for binding (e.g., a hybridizing to) a polyT or polyU oligonucleotide. In some embodiments, the target region includes at least 10 (e.g., at least 11, 12, 13, 14, 15, 16, 17, 18, 19, 20, 21, 22, 23, 24, 25, 26, 27, 28, 29, 30, or more, e.g., at least 25) consecutive adenines or deoxyadenines. In some embodiments, the target region contains a poly(A)₂₅.

### Particles

The oligonucleotides described herein may be conjugated (e.g., directly or indirectly) to a particle, e.g., a magnetic particle or a bead. In some embodiments, the oligonucleotide is conjugated to a plurality of particles. In some embodiments, a particle is conjugated to a plurality of oligonucleotides.

Magnetic particles include at least one component that is responsive to a magnetic force. A magnetic particle may be entirely magnetic or may contain components that are non-magnetic. A magnetic particle may be a magnetic bead, e.g., a substantially spherical magnetic bead. The magnetic particle may be entirely magnetic or may contain one or more magnetic cores surrounded by one or more additional materials, such as, for example, one or more functional groups and/or modifications for binding one or more target molecules. In some examples, a magnetic particle may contain a magnetic component and a surface modified with one or more silanol groups. Magnetic particles of this type may be used for binding target nucleic acid molecules.

A particle, e.g., a magnetic particle or a bead, may be porous, non-porous, hollow, solid, semi-solid, semi-fluidic, fluidic, and/or a combination thereof. In some instances, a particle, e.g., a bead, may be dissolvable or degradable. In some cases, a particle, e.g., a bead, may not be degradable. In some embodiments, the bead is composed of crosslinked agarose, e.g., SEPHAROSE® agarose.

A particle, e.g., a magnetic particle or a bead, may include natural and/or synthetic materials. For example, a particle, e.g., a bead, can include a natural polymer, a synthetic polymer or both natural and synthetic polymers. Examples of natural polymers include proteins and sugars such as deoxyribonucleic acid, rubber, cellulose, starch (e.g., amylose, amylopectin), proteins, enzymes, polysaccharides, silks, polyhydroxyalkanoates, chitosan, dextran, collagen, carrageenan, ispaghula, acacia, agar, gelatin, shellac, sterculia gum, xanthan gum, corn sugar gum, guar gum, gum karaya, agarose, alginic acid, alginate, or natural polymers thereof. Examples of synthetic polymers include acrylics, nylons, silicones, spandex, viscose rayon, polycarboxylic acids, polyvinyl acetate, polyacrylamide, polyacrylate, polyethylene glycol, polyurethanes, polylactic acid, silica, polystyrene, polyacrylonitrile, polybutadiene, polycarbonate, polyethylene, polyethylene terephthalate, poly(chlorotrifluoroethylene), poly(ethylene oxide), poly(ethylene terephthalate), polyethylene, polyisobutylene, poly(methyl methacrylate), poly(oxymethylene), polyformaldehyde, polypropylene, polystyrene, poly(tetrafluoroethylene), poly(vinyl acetate), poly(vinyl alcohol), poly(vinyl chloride), poly(vinylidene dichloride), poly(vinylidene difluoride), poly(vinyl fluoride) and/or combinations (e.g., co-polymers) thereof. Beads may also be formed from materials other than polymers, including lipids, micelles, ceramics, glass-ceramics, material composites, metals, other inorganic materials, and others.

Cross-linking may be permanent or reversible, depending upon the particular cross-linker used. Reversible cross-linking may allow for the polymer to linearize or dissociate under appropriate conditions. In some cases, reversible cross-linking may also allow for reversible attachment of a material bound to the surface of a bead.

Particles, e.g., beads or magnetic particles, may be of uniform size or heterogeneous size. In some cases, the diameter of a particle, e.g., a bead, may be at least about 1 μm, 5 μm, 10 μm, 20 μm, 30 μm, 40 μm, 50 μm, 60 μm, 70 μm, 80 μm, 90 μm, 100 μm, 250 μm, 500 μm, 1 mm, 2 mm, 3 mm, 4 mm, 5 mm, 6 mm, 7 mm, 8 mm, 9 mm, 10 mm, or greater. In some cases, a particle, e.g., a bead, may have a diameter of less than about 1 μm, 5 μm, 10 μm, 20 μm, 30 μm, 40 μm, 50 μm, 60 μm, 70 μm, 80 μm, 90 μm, 100 μm, 250 μm, 500 μm, 1 mm, 2 mm, 3 mm, 4 mm, 5 mm, 6 mm, 7 mm, 8 mm, 9 mm, 10 mm or less. In some cases, a particle, e.g., a bead, may have a diameter in the range of about 40-75 μm, 30-75 μm, 20-75 μm, 40-85 μm, 40-95 μm, 20-100 μm, 10-100 μm, 1-100 μm, 20-250 μm, or 20-500 μm, 500 μm-1 mm, 1 mm-2 mm, 1-5 mm, or 1-10 mm.

Particles may be of any suitable shape. Examples of particles, e.g., magnetic particles or beads, shapes include, but are not limited to, spherical, non-spherical, oval, oblong, amorphous, circular, cylindrical, and variations thereof.

### Linkers

In some embodiments, a linker is used to conjugate two or more components used in a composition or method described herein. For example, a linker may be used to conjugate an oligonucleotide to a particle (e.g., a bead), a target region to a linear polyribonucleotide or any combination or variation thereof. In some embodiments, the target region is conjugated to the linear polyribonucleotide with a chemical linker. In some embodiments, the oligonucleotide is conjugated to the particle with a chemical linker. The chemical linker may be conjugated to a 3′ end or a 5′ end of the oligonucleotide. Alternatively, the chemical linker may be conjugated to an interior region of the oligonucleotide. The particle may be, for example, a magnetic particle or a bead. The bead may be, e.g., a crosslinked agarose, e.g., a SEPHAROSE® bead. In some embodiments, an oligonucleotide is conjugated directly to a particle (e.g., a bead, e.g., a magnetic bead or crosslinked agarose, e.g., SEPHAROSE® bead).

A chemical linker provides space, rigidity, and/or flexibility between, for example, an oligonucleotide and a particle or a target region and a linear polyribonucleotide. In some embodiments, a linker may be a bond, e.g., a covalent bond, e.g., an amide bond, a disulfide bond, a C—O bond, a C—N bond, a N—N bond, a C—S bond, or any kind of bond created from a chemical reaction, e.g., chemical conjugation. In some embodiments, a linker includes no more than 250 atoms (e.g., 1-2, 1-4, 1-6, 1-8, 1-10, 1-12, 1-14, 1-16, 1-18, 1-20, 1-25, 1-30, 1-35, 1-40, 1-45, 1-50, 1-55, 1-60, 1-65, 1-70, 1-75, 1-80, 1-85, 1-90, 1-95, 1-100, 1-110, 1-120, 1-130, 1-140, 1-150, 1-160, 1-170, 1-180, 1-190, 1-200, 1-210, 1-220, 1-230, 1-240, or 1-250 atom(s); 250, 240, 230, 220, 210, 200, 190, 180, 170, 160, 150, 140, 130, 120, 110, 100, 95, 90, 85, 80, 75, 70, 65, 60, 55, 50, 45, 40, 35, 30, 28, 26, 24, 22, 20, 18, 16, 14, 12, 10, 9, 8, 7, 6, 5, 4, 3, 2, or 1 atom(s)). In some embodiments, a linker includes no more than 250 non-hydrogen atoms (e.g., 1-2, 1-4, 1-6, 1-8, 1-10, 1-12, 1-14, 1-16, 1-18, 1-20, 1-25, 1-30, 1-35, 1-40, 1-45, 1-50, 1-55, 1-60, 1-65, 1-70, 1-75, 1-80, 1-85, 1-90, 1-95, 1-100, 1-110, 1-120, 1-130, 1-140, 1-150, 1-160, 1-170, 1-180, 1-190, 1-200, 1-210, 1-220, 1-230, 1-240, or 1-250 non-hydrogen atom(s); 250, 240, 230, 220, 210, 200, 190, 180, 170, 160, 150, 140,130, 120, 110, 100, 95, 90, 85, 80, 75, 70, 65, 60, 55, 50, 45, 40, 35, 30, 28, 26, 24, 22, 20, 18, 16, 14, 12, 10, 9, 8, 7, 6, 5, 4, 3, 2, or 1 non-hydrogen atom(s)). In some embodiments, the backbone of a linker includes no more than 250 atoms (e.g., 1-2, 1-4, 1-6, 1-8, 1-10, 1-12, 1-14, 1-16, 1-18, 1-20, 1-25, 1-30, 1-35, 1-40, 1-45, 1-50, 1-55, 1-60, 1-65, 1-70, 1-75, 1-80, 1-85, 1-90, 1-95, 1-100, 1-110, 1-120, 1-130, 1-140, 1-150, 1-160, 1-170, 1-180, 1-190, 1-200, 1-210, 1-220, 1-230, 1-240, or 1-250 atom(s); 250, 240, 230, 220, 210, 200, 190, 180, 170, 160, 150, 140, 130, 120, 110, 100, 95, 90, 85, 80, 75, 70, 65, 60, 55, 50, 45, 40, 35, 30, 28, 26, 24, 22, 20, 18, 16, 14, 12, 10, 9, 8, 7, 6, 5, 4, 3, 2, or 1 atom(s)). The “backbone” of a linker refers to the atoms in the linker that together form the shortest path from one part of the conjugate to another part of the conjugate. The atoms in the backbone of the linker are directly involved in linking one part of the conjugate to another part of the conjugate. For examples, hydrogen atoms attached to carbons in the backbone of the linker are not considered as directly involved in linking one part of the conjugate to another part of the conjugate.

In some embodiments, a linker may include a synthetic group derived from, e.g., a synthetic polymer (e.g., a polyethylene glycol (PEG) polymer). The chemical linker may include, e.g., triethylene glycol (TEG). In some embodiments, a linker may include one or more amino acid residues. In some embodiments, a linker may be an amino acid sequence (e.g., a 1-25 amino acid, 1-10 amino acid, 1-9 amino acid, 1-8 amino acid, 1-7 amino acid, 1-6 amino acid, 1-5 amino acid, 1-4 amino acid, 1-3 amino acid, 1-2 amino acid, or 1 amino acid sequence). In some embodiments, a linker may include one or more optionally substituted C₁-C₂₀ alkylene, optionally substituted C₁-C₂₀ heteroalkylene (e.g., a PEG unit), optionally substituted C₂-C₂₀ alkenylene (e.g., C2 alkenylene), optionally substituted C₂-C₂₀ heteroalkenylene, optionally substituted C₂-C₂₀ alkynylene, optionally substituted C₂-C₂₀ heteroalkynylene, optionally substituted C₃-C₂₀cycloalkylene (e.g., cyclopropylene, cyclobutylene), optionally substituted C₂-C₂₀ heterocycloalkylene, optionally substituted C₄-C₂₀ cycloalkenylene, optionally substituted C₄-C₂₀ heterocycloalkenylene, optionally substituted C₈-C₂₀ cycloalkynylene, optionally substituted C₁-C₂₀ heterocycloalkynylene, optionally substituted C₅-C₁₈ arylene (e.g., C₆ arylene), optionally substituted C₃-C₁₅ heteroarylene (e.g., imidazole, pyridine), O, S, NRi (Ri is H, optionally substituted C₁-C₂₀ alkyl, optionally substituted C₁-C₂₀ heteroalkyl, optionally substituted C₂-C₂₀ alkenyl, optionally substituted C₂-C₂₀ heteroalkenyl, optionally substituted C₂-C₂₀ alkynyl, optionally substituted C₂-C₂₀ heteroalkynyl, optionally substituted C₃-C₂₀ cycloalkyl, optionally substituted C₂-C₂₀ heterocycloalkyl, optionally substituted C₄-C₂₀ cycloalkenyl, optionally substituted C₄-C₂₀ heterocycloalkenyl, optionally substituted C₈-C₂₀ cycloalkynyl, optionally substituted C₈-C₂₀ heterocycloalkynyl, optionally substituted C₅-C₁₅ aryl, or optionally substituted C₃-C₁₅ heteroaryl), P, carbonyl, thiocarbonyl, sulfonyl, phosphate, phosphoryl, or imino.

Covalent conjugation of two or more components in a conjugate using a linker may be accomplished using well-known organic chemical synthesis techniques and methods. Complementary functional groups on two components may react with each other to form a covalent bond. Examples of complementary reactive functional groups include, but are not limited to, e.g., maleimide and cysteine, amine and activated carboxylic acid, thiol and maleimide, activated sulfonic acid and amine, isocyanate and amine, azide and alkyne, and alkene and tetrazine. Site-specific conjugation to a polypeptide may accomplished using techniques known in the art.

### Resins

In some embodiments, the methods described herein include using a resin with a plurality of particles conjugated to an oligonucleotide that hybridizes to the target region. The methods may include using a column that includes the resin. The method may include collecting an eluate (e.g., not bound to the resin) that includes a portion of the sample that is not hybridized to the oligonucleotide from the plurality of polyribonucleotides in the sample. In some embodiments, the resin includes cross-linked poly[styrene-divinylbenzene], agarose, or SEPHAROSE® agarose.

Compositions and methods of the invention can use a surface linked to the oligonucleotide that contains a sequence configured to hybridize to a target region. Such oligonucleotides may include, e.g., polyT, polyU, or polyT/U. The surface of the resin refers to a part of a support structure (e.g., a substrate) that is accessible to contact with one or more reagents or oligonucleotides. The shape, form, materials, and modifications of the surface of the resin can be selected from a range of options depending on the application. In one embodiment, the surface of the resin is SEPHAROSE® agarose. In one embodiment, the surface of the resin is agarose

The surface of the resin can be substantially flat or planar. Alternatively, the surface of the resin can be rounded or contoured. Exemplary contours that can be included on a surface of the resin are wells, depressions, pillars, ridges, channels or the like.

Exemplary materials that can be used as a surface of the resin include, but are not limited to acrylics, carbon (e.g., graphite, carbon-fiber), cellulose (e.g., cellulose acetate), ceramics, controlled-pore glass, cross-linked polysaccharides (e.g., agarose or SEPHAROSE® agarose), gels, glass (e.g., modified or functionalized glass), gold (e.g., atomically smooth Au(I 11)), graphite, inorganic glasses, inorganic polymers, latex, metal oxides (e.g., SiO₂, TiO₂, stainless steel), metalloids, metals (e.g., atomically smooth Au(I 11)), mica, molybdenum sulfides, nanomaterials (e.g., highly oriented pyrolitic graphite (HOPG) nanosheets), nitrocellulose, NYLON™, optical fiber bundles, organic polymers, paper, plastics, polacryloylmorpholide, poly(4-methylbutene), polyethylene terephthalate), poly(vinyl butyrate), polybutylene, polydimethylsiloxane (PDMS), polyethylene, polyformaldehyde, polymethacrylate, polypropylene, polysaccharides, polystyrene, polyurethanes, polyvinylidene difluoride (PVDF), quartz, rayon, resins, rubbers, semiconductor material, silica, silicon (e.g., surface-oxidized silicon), sulfide, and TEFLON™. A single material or mixture of several different materials can form a resin useful in the invention.

In some embodiments, a surface of the resin includes a polymer.

In some embodiments, a surface of the resin includes SEPHAROSE® agarose. An example is shown below, where n is any positive integer:

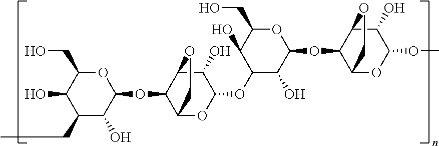

In some embodiments, a surface of the resin includes agarose. An example is shown below, where n is a positive integer:

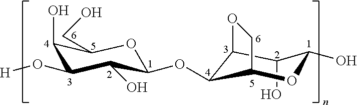

Structure of agarose: D-galactose and 3,6-anhydro-a-L-galactopyranose repeating Unit.

In some embodiments, a surface of the resin includes a polystyrene-based polymer. A polystyrene divinyl benzene copolymer synthesis schematic is shown below:

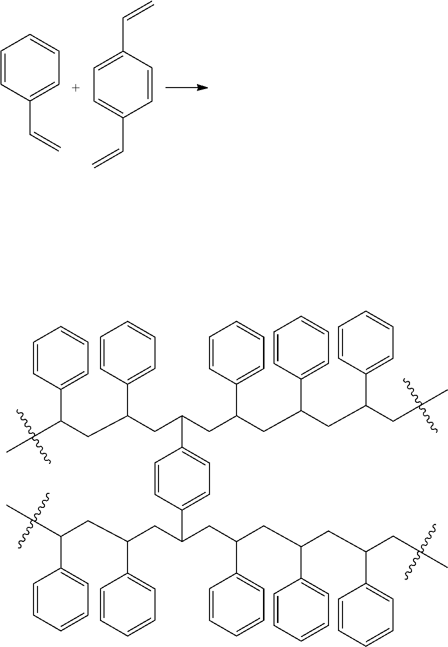

In some embodiments, a surface of the resin includes an acrylic based polymer. Poly (methylmethacrylate) is an example shown below, wherein n is any positive integer:

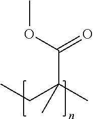

In some embodiments, a surface of the resin includes a dextran-based polymer. A Dextran example is shown below:

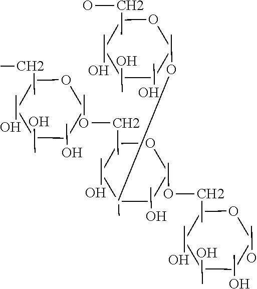

In some embodiments, a surface of the resin includes silica. An example is shown below:

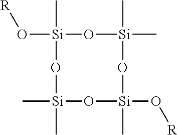

In some embodiments, a surface of the resin includes a polyacrylamide. An example cross-linked to N-N-methylenebisacrylamide is shown below:

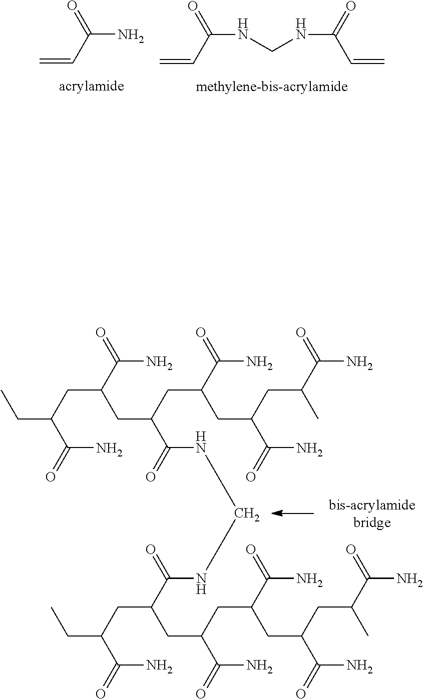

In some embodiments, a surface of the resin includes tentacle-based phases, e.g., methacrylate based.

A number of surfaces known in the art are suitable for use with the methods of the invention. Suitable surfaces may include materials including but not limited to borosilicate glass, agarose, SEPHAROSE agarose, magnetic beads, polystyrene, polyacrylamide, membranes, silica, semiconductor materials, silicon, organic polymers, ceramic, glass, metal, plastic polycarbonate, polycarbonate, polyethylene, polyethyleneglycol terephthalate, polymethylmethacrylate, polypropylene, polyvinylacetate, polyvinylchloride, polyvinylpyrrolidinone, and soda-lime glass.

In one embodiment, the surface of the resin is modified to contain channels, patterns, layers, or other configurations (e.g., a patterned surface). The surface can be in the form of a bead, box, column, cylinder, disc, dish (e.g., glass dish, PETRI dish), fiber, film, filter, microtiter plate (e.g., 96-well microtiter plate), multi-bladed stick, net, pellet, plate, ring, rod, roll, sheet, slide, stick, tray, tube, or vial. The surface can be a singular discrete body (e.g., a single tube, a single bead), any number of a plurality of surface bodies (e.g., a rack of 10 tubes, several beads), or combinations thereof (e.g., a tray includes a plurality of microtiter plates, a column filled with beads, a microtiter plate filed with beads).

In some embodiments, a surface can include a membrane-based resin matrix. In some embodiments, the surface of the resin includes a porous resin or a non-porous resin. Examples of porous resins can include additional agarose-based resins (e.g., cyanogen bromide activated SEPHAROSE® agarose (GE); WorkBeads™ 40 ACT and WorkBeads 40/10000 ACT (Bioworks)), methacrylate: (Tosoh 650M derivatives etc.), polystyrene divinylbenzene (Life Tech Poros media/GE Source media), fractogel, polyacrylamide, silica, controlled pore glass, dextran derivatives, acrylamide derivatives, additional polymers, and combinations thereof.

In some embodiments, a surface can include one or more pores. In some embodiments, pore sizes can be from 300 to 8,000 Angstroms, e.g., 500 to 4,000 Angstroms in size.

A resin as described herein includes a plurality of particles. Examples of particle sizes are 5 μm-500 μm, 20 μm-300 μm, and 50 μm-200 μm. In some embodiments, particle size can be 50 μm, 60 μm, 70 μm, 80 μm, 90 μm, 100 μm, 110 μm, 120 μm, 130 μm, 140 μm, 150 μm, 160 μm, 170 μm, 180 μm, 190 μm, or 200 μm.

An oligonucleotide can be immobilized, coated on, bound to, stuck, adhered, or attached to any of the forms of surfaces described herein (e.g., bead, box, column, cylinder, disc, dish (e.g., glass dish, PETRI dish), fiber, film, filter, microtiter plate (e.g., 96-well microtiter plate), multi-bladed stick, net, pellet, plate, ring, rod, roll, sheet, slide, stick, tray, tube, or vial).

In one embodiment, the surface is modified to contain chemically modified sites that can be used to attach (e.g., either covalently or non-covalently) the oligonucleotide to discrete sites or locations on the surface. Chemically modified sites include for example, the addition of a pattern of chemical functional groups including amino groups, carboxy groups, oxo groups and thiol groups, which can be used to covalently attach the oligonucleotide, which generally also contain corresponding reactive functional groups. Examples of surface functionalization are amino derivatives, thiol derivatives, aldehyde derivatives, formyl derivatives, azide derivatives (click chemistry), biotin derivatives, alkyne derivatives, hydroxyl derivatives, activated hydroxyls or derivatives, carboxylate derivatives, activated carboxylate derivates, activated carbonates, activated esters, NHS ester (succinimidyl), NHS carbonate (succinimidyl), Imidoester or derivated, cyanogen bromide derivatives, maleimide derivatives, haloacteyl derivatives, iodoacetamide/iodoacetyl derivatives, epoxide derivatives, streptavidin derivatives, tresyl derivatives, diene/conjugated diene derivatives (Diels-Alder type reaction), alkene derivatives, substituted phosphate derivatives, bromohydrin/halohydrin, substituted disulfides, pyridyl-disulfide derivatives, aryl azides, acyl azides, azlactone, hydrazide derivatives, halobenzene derivatives, nucleoside derivatives, branching/multi-functional linkers, dendrimeric functionalities, nucleoside derivatives, or any combination thereof.

In some embodiments, the binding capacity of the linked surface can be at least 1 mg/mL, 5 mg/mL, 10 mg/mL, 20 mg/mL, 30 mg/mL, 40 mg/mL, 50 mg/mL, or more.

In some embodiments, a column that includes the resin is configured bind at least 500 μg (e.g., at least 600 μg, 700 μg, 800 μg, 900 μg, 1 mg, 2 mg, 3 mg, 4 mg, 5 mg, 6 mg, 7 mg, 8 mg, 9 mg, 10 mg, 20 mg, 30 mg, 40 mg, 50 mg, 60 mg, 70 mg, 80 mg, 90 mg, 100 mg, 200 mg, 300 mg, 400 mg, 500 mg, 600 mg, 700 mg, 800 mg, 900 mg, 1,000 mg, or more) of polyribonucleotides, e.g., with the target region. In some embodiments, the column is configured to bind from 500 μg to 1,000 mg of polyribonucleotides, e.g., with the target region

### Compositions

As described herein, the invention features a composition that includes a population of polyribonucleotides produced by a method as described herein. The population may include, e.g., a circular polyribonucleotide lacking a target region, and the circular polyribonucleotide includes at least 1% (e.g., at least 5%, e.g., at least 10%, at least 20%, at least 30%, at least 40%, at least 50%, 60%, 70%, 80%, 90%, 95%, 97%, 99%, or more) (mol/mol) of the total polyribonucleotides in the composition. In some embodiments, the population has less than 50% (e.g., less than 40%, 30%, 20%, 10%, 5%, 4%, 3%, 2%, or 1%) (mol/mol) linear polyribonucleotides in the composition.

In other embodiments, the population includes, e.g., a polyribonucleotide in a first conformation having the target region and the polyribonucleotide in a second conformation having the target region, and the polyribonucleotide in the first conformation includes at least 1%, e.g., at least 5%, e.g., at least 10%, at least 20%, at least 30%, or at least 40% (e.g., at least 50%, 60%, 70%, 80%, 90%, 95%, 97%, 99%, or more) (mol/mol) of the total polyribonucleotides in the composition.

In some embodiments as described herein, the invention features a composition that includes a mixture of polyribonucleotides. A first subset of the mixture includes a circular polyribonucleotide lacking a target region, and a second subset of the plurality of the polyribonucleotides includes a linear polyribonucleotide having the target region. The first subset includes at least 1%, e.g., at least 5%, e.g., at least 10%, at least 20%, at least 30%, or at least 40% (e.g., at least 50%, 60%, 70%, 80%, 90%, 95%, 97%, 99%, or more) (mol/mol) of the total polyribonucleotides in the composition. In some embodiments, the linear polyribonucleotides include a variety of distinct linear polyribonucleotide species, e.g., each containing the target region.

In some embodiments as described herein, the invention features a composition that includes a polyribonucleotide having a target region and an oligonucleotide configured to hybridize to the target region, wherein the oligonucleotide is conjugated to a particle, e.g., via a linker.

In some embodiments of any of the compositions as described herein, the linear polyribonucleotide includes an intron or portion thereof. The target region may be located 5′ or 3′ to the intron or portion thereof.

In some embodiments, the polyribonucleotide may be a modified polyribonucleotide.

In an embodiment, a circular polyribonucleotide preparation (e.g., a circular polyribonucleotide pharmaceutical preparation or composition or an intermediate in the production of the circular polyribonucleotide preparation) is at least 30% (w/w), 40% (w/w), 50% (w/w), 60% (w/w), 70% (w/w), 80% (w/w), 85% (w/w), 90% (w/w), 91% (w/w), 92% (w/w), 93% (w/w), 94% (w/w), 95% (w/w), 96% (w/w), 97% (w/w), 98% (w/w), 99% (w/w), or 100% (w/w) pure on a mass basis. Purity may be measured by any one of a number of analytical techniques known to one skilled in the art, such as, but not limited to, the use of separation technologies such as chromatography (using a column, using a paper, using a gel, using HPLC, using UHPLC, etc., or by IC, by SEC, by reverse phase, by anion exchange, by mixed mode, etc.) or electrophoresis (UREA PAGE, chip-based, polyacrylamide gel, RNA, capillary, c-IEF, etc.) with or without pre- or post-separation derivatization methodologies using detection techniques based on mass spectrometry, UV-visible, fluorescence, light scattering, refractive index, or that use silver or dye stains or radioactive decay for detection. Alternatively, purity may be determined without the use of a separation technology by mass spectrometry, by microscopy, by circular dichroism (CD) spectroscopy, by UV or UV-vis spectrophotometry, by fluorometry (e.g., Qubit), by RNAse H analysis, by surface plasmon resonance (SPR), or by methods that utilize silver or dye stains or radioactive decay for detection.

In some embodiments, purity can be measured by biological test methodologies (e.g., cell-based or receptor-based tests). In some embodiments, at least 30% (w/w), 40% (w/w), 50% (w/w), 60% (w/w), 70% (w/w), 80% (w/w), 85% (w/w), 90% (w/w), 91% (w/w), 92% (w/w), 93% (w/w), 94% (w/w), 95% (w/w), 96% (w/w), 97% (w/w), 98% (w/w), 99% (w/w) or 100% (w/w) of the total of mass ribonucleotide in the a preparation described herein is contained in circular polyribonucleotide molecules. The percent may be measured by any one of a number of analytical techniques known to one skilled in the art such as, but not limited to, the use of a separation technology such as chromatography (using a column, using a paper, using a gel, using HPLC, using UHPLC, etc., or by IC, by SEC, by reverse phase, by anion exchange, by mixed mode, etc.) or electrophoresis (UREA PAGE, chip-based, polyacrylamide gel, RNA, capillary, c-IEF, etc.) with or without pre- or post-separation derivatization methodologies using detection techniques based on mass spectrometry, UV-visible, fluorescence, light scattering, refractive index, or that use silver or dye stains or radioactive decay for detection. Alternatively, purity may be determined without the use of separation technologies by mass spectrometry, by microscopy, by circular dichroism (CD) spectroscopy, by UV or UV-vis spectrophotometry, by fluorometry (e.g., Qubit), by RNAse H analysis, by surface plasmon resonance (SPR), or by methods that utilize silver or dye stains or radioactive decay for detection.

In an embodiment, a circular polyribonucleotide preparation (e.g., a circular polyribonucleotide pharmaceutical preparation or composition or an intermediate in the production of the circular polyribonucleotide preparation) has a circular polyribonucleotide concentration of at least 0.1 ng/mL, 0.5 ng/mL, 1 ng/mL, 5 ng/mL, 10 ng/mL, 50 ng/mL, 0.1 μg/mL, 0.5 μg/mL, 1 μg/mL, 2 μg/mL, 5 μg/mL, 10 μg/mL, 20 μg/mL, 30 μg/mL, 40 μg/mL, 50 μg/mL, 60 μg/mL, 70 μg/mL, 80 μg/mL, 100 μg/mL, 200 μg/mL, 300 μg/mL, 500 μg/mL, 1000 μg/mL, 5000 μg/mL, 10,000 μg/mL, 100,000 μg/mL, 200 mg/mL, 300 mg/mL, 400 mg/mL, 500 mg/mL, 600 mg/mL, 650 mg/mL, 700 mg/mL, or 750 mg/mL. In an embodiment, a circular polyribonucleotide preparation (e.g., a circular polyribonucleotide pharmaceutical preparation or composition or an intermediate in the production of the circular polyribonucleotide preparation) is substantially free of mononucleotide or has a mononucleotide content of no more than 1 μg/ml, 10 μg/ml, 0.1 ng/ml, 1 ng/ml, 5 ng/ml, 10 ng/ml, 15 ng/ml, 20 ng/ml, 25 ng/ml, 30 ng/ml, 35 ng/ml, 40 ng/ml, 50 ng/ml, 60 ng/ml, 70 ng/ml, 80 ng/ml, 90 ng/ml, 100 ng/ml, 200 ng/ml, 300 ng/ml, 400 ng/ml, 500 ng/ml, 1000 μg/mL, 5000 μg/mL, 10,000 μg/mL, or 100,000 μg/mL. In an embodiment, a circular polyribonucleotide preparation (e.g., a circular polyribonucleotide pharmaceutical preparation or composition or an intermediate in the production of the circular polyribonucleotide preparation) has a mononucleotide content from the limit of detection up to 1 μg/ml, 10 μg/ml, 0.1 ng/ml, 1 ng/ml, 5 ng/ml, 10 ng/ml, 15 ng/ml, 20 ng/ml, 25 ng/ml, 30 ng/ml, 35 ng/ml, 40 ng/ml, 50 ng/ml, 60 ng/ml, 70 ng/ml, 80 ng/ml, 90 ng/ml, 100 ng/ml, 200 ng/ml, 300 ng/ml, 400 ng/ml, 500 ng/ml, 1000 μg/mL, 5000 μg/mL, 10,000 μg/mL, or 100,000 μg/mL.

In an embodiment, a circular polyribonucleotide preparation (e.g., a circular polyribonucleotide pharmaceutical preparation or composition or an intermediate in the production of the circular polyribonucleotide preparation) has mononucleotide content no more than 0.1% (w/w), 0.2% (w/w), 0.3% (w/w), 0.4% (w/w), 0.5% (w/w), 0.6% (w/w), 0.7% (w/w), 0.8% (w/w), 0.9% (w/w), 1% (w/w), 2% (w/w), 3% (w/w), 4% (w/w), 5% (w/w), 6% (w/w), 7% (w/w), 8% (w/w), 9% (w/w), 10% (w/w), 15% (w/w), 20% (w/w), 25% (w/w), 30% (w/w), or any percentage therebetween of total nucleotides on a mass basis, wherein total nucleotide content is the total mass of deoxyribonucleotide molecules and ribonucleotide molecules.

In an embodiment, a circular polyribonucleotide preparation (e.g., a circular polyribonucleotide pharmaceutical preparation or composition or an intermediate in the production of the circular polyribonucleotide preparation) has a linear RNA content, e.g., linear RNA counterpart or RNA fragments, of no more than 1 ng/ml, 5 ng/ml, 10 ng/ml, 15 ng/ml, 20 ng/ml, 25 ng/ml, 30 ng/ml, 35 ng/ml, 40 ng/ml, 50 ng/ml, 6 Ong/ml, 70 ng/ml, 80 ng/ml, 90 ng/ml, 100 ng/ml, 200 ng/ml, 300 ng/ml, 400 ng/ml, 500 ng/ml, 600 ng/ml, 1 μg/ml, 10 μg/ml, 50 μg/ml, 100 μg/ml, 200 g/ml, 300 μg/ml, 400 μg/ml, 500 μg/ml, 600 μg/ml, 700 μg/ml, 800 μg/ml, 900 μg/ml, 1 mg/ml, 1.5 mg/ml, 2 mg/ml, 5 mg/mL, 10 mg/mL, 50 mg/mL, 100 mg/mL, 200 mg/mL, 300 mg/mL, 400 mg/mL, 500 mg/mL, 600 mg/mL, 650 mg/mL, 700 mg/mL, or 750 mg/mL. In an embodiment, a circular polyribonucleotide preparation (e.g., a circular polyribonucleotide pharmaceutical preparation or composition or an intermediate in the production of the circular polyribonucleotide preparation) has a linear RNA content, e.g., linear RNA counterpart or RNA fragments, from the limit of detection of up to 1 ng/ml, 5 ng/ml, 10 ng/ml, 15 ng/ml, 20 ng/ml, 25 ng/ml, 30 ng/ml, 35 ng/ml, 40 ng/ml, 50 ng/ml, 6 Ong/ml, 70 ng/ml, 80 ng/ml, 90 ng/ml, 100 ng/ml, 200 ng/ml, 300 ng/ml, 400 ng/ml, 500 ng/ml, 600 ng/ml, 1 μg/ml, 10 μg/ml, 50 μg/ml, 100 μg/ml, 200 g/ml, 300 μg/ml, 400 μg/ml, 500 μg/ml, 600 μg/ml, 700 μg/ml, 800 μg/ml, 900 μg/ml, 1 mg/ml, 1.5 mg/ml, 2 mg/ml, 5 mg/ml, 10 mg/ml, 50 mg/ml, 100 mg/ml, 200 mg/ml, 300 mg/ml, 400 mg/ml, 500 mg/ml, 600 mg/ml, 650 mg/ml, 700 mg/ml, or 750 mg/ml.

In an embodiment, a circular polyribonucleotide preparation (e.g., a circular polyribonucleotide pharmaceutical preparation or composition or an intermediate in the production of the circular polyribonucleotide preparation) has a nicked RNA content of no more than 10% (w/w), 9.9% (w/w), 9.8% (w/w), 9.7% (w/w), 9.6% (w/w), 9.5% (w/w), 9.4% (w/w), 9.3% (w/w), 9.2% (w/w), 9.1% (w/w), 9% (w/w), 8% (w/w), 7% (w/w), 6% (w/w), 5% (w/w), 4% (w/w), 3% (w/w), 2% (w/w), 1% (w/w), 0.5% (w/w), or 0.1% (w/w), or percentage therebetween. In an embodiment, a circular polyribonucleotide preparation (e.g., a circular polyribonucleotide pharmaceutical preparation or composition or an intermediate in the production of the circular polyribonucleotide preparation) has a nicked RNA content that as low as zero or is substantially free of nicked RNA.

In an embodiment, a circular polyribonucleotide preparation (e.g., a circular polyribonucleotide pharmaceutical preparation or composition or an intermediate in the production of the circular polyribonucleotide preparation) has a combined linear RNA and nicked RNA content of no more than 30% (w/w), 25% (w/w), 20% (w/w), 15% (w/w), 10% (w/w), 9% (w/w), 8% (w/w), 7% (w/w), 6% (w/w), 5% (w/w), 4% (w/w), 3% (w/w), 2% (w/w), 1% (w/w), 0.5% (w/w), or 0.1% (w/w), or percentage therebetween.

In an embodiment, a circular polyribonucleotide preparation (e.g., a circular polyribonucleotide pharmaceutical preparation or composition or an intermediate in the production of the circular polyribonucleotide preparation) has a combined nicked RNA and linear RNA content that is as low as zero or is substantially free of nicked and linear RNA.

In some embodiments, a circular polyribonucleotide preparation (e.g., a circular polyribonucleotide pharmaceutical preparation or composition or an intermediate in the production of the circular polyribonucleotide preparation) has a linear RNA content, e.g., linear RNA counterpart or RNA fragments, of no more than the detection limit of analytical methodologies, such as methods utilizing mass spectrometry, UV spectroscopic or fluorescence detectors, light scattering techniques, surface plasmon resonance (SPR) with or without the use of methods of separation including HPLC, by HPLC, chip or gel based electrophoresis with or without using either pre or post separation derivatization methodologies, methods of detection that use silver or dye stains or radioactive decay, or microscopy, visual methods or a spectrophotometer.

In an embodiment, a circular polyribonucleotide preparation (e.g., a circular polyribonucleotide pharmaceutical preparation or composition or an intermediate in the production of the circular polyribonucleotide preparation) has no more than 0.1% (w/w), 1% (w/w), 2% (w/w), 3% (w/w), 4% (w/w), 5% (w/w), 6% (w/w), 7% (w/w), 8% (w/w), 9% (w/w), 10% (w/w), 15% (w/w), 20% (w/w), 25% (w/w), 30% (w/w), 35% (w/w), 40% (w/w), 45% (w/w), 50% (w/w) of linear RNA, e.g., as measured by the methods in Example 2.

In some embodiments, the linear polyribonucleotide molecules of the circular polyribonucleotide preparation include the linear counterpart or a fragment thereof of the circular polyribonucleotide molecule. In some embodiments, the linear polyribonucleotide molecules of the circular polyribonucleotide preparation include the linear counterpart (e.g., a pre-circularized version). In some embodiments, the linear polyribonucleotide molecules of the circular polyribonucleotide preparation include a non-counterpart or fragment thereof to the circular polyribonucleotide. In some embodiments, the linear polyribonucleotide molecules of the circular polyribonucleotide preparation include a non-counterpart to the circular polyribonucleotide. In some embodiments, the linear polyribonucleotide molecules include a combination of the counterpart of the circular polyribonucleotide and a non-counterpart or fragment thereof of the circular polyribonucleotide. In some embodiments, the linear polyribonucleotide molecules include a combination of the counterpart of the circular polyribonucleotide and a non-counterpart of the circular polyribonucleotide. In some embodiments, a linear polyribonucleotide molecule fragment is a fragment that is at least 1, 2, 3, 4, 5, 6, 7, 8, 9, 10, 20, 30, 40, 50, 60, 70, 80, 90, 100, 200, 300, 400, 500, 1000, 2000, 3000, 4000, 5000, 6000, 7000, 8000, 9000, 10000, 11000, 12000, or more nucleotides in length, or any nucleotide number therebetween.

In some embodiments, a circular polyribonucleotide preparation (e.g., a circular polyribonucleotide pharmaceutical preparation or composition or an intermediate in the production of the circular polyribonucleotide preparation) has an A260/A280 absorbance ratio from about 1.6 to about 2.3, e.g., as measured by spectrophotometer. In some embodiments, the A260/A280 absorbance ratio is about 1.4, 1.5, 1.6, 1.7, 1.8, 1.9, 2.0, 2.1, 2.2, 2.3, 2.4, 2.5, or any number therebetween. In some embodiments, a circular polyribonucleotide (e.g., a circular polyribonucleotide pharmaceutical preparation or composition or an intermediate in the production of the circular polyribonucleotide) has an A260/A280 absorbance ratio greater than about 1.8, e.g., as measured by spectrophotometer. In some embodiments, the A260/A280 absorbance ratio is about 1.6, 1.7, 1.8, 1.9, 2.0, 2.1, 2.2, 2.3, 2.4, 2.5, 2.6, 2.7, 2.8, 2.9, or greater.

In some embodiments, a circular polyribonucleotide preparation (e.g., a circular polyribonucleotide pharmaceutical preparation or composition or an intermediate in the production of the circular polyribonucleotide preparation) is substantially free of an impurity or byproduct. In various embodiments, the level of at least one impurity or byproduct in a composition including the circular polyribonucleotide is reduced by at least 30% (w/w), at least 40% (w/w), at least 50% (w/w), at least 60% (w/w), at least 70% (w/w), at least 80% (w/w), at least 90% (w/w), or at least 95% (w/w) as compared to that of the composition prior to purification or treatment to remove the impurity or byproduct. In some embodiments, the level of at least one process-related impurity or byproduct is reduced by at least 30% (w/w), at least 40% (w/w), at least 50% (w/w), at least 60% (w/w), at least 70% (w/w), at least 80% (w/w), at least 90% (w/w), or at least 95% (w/w) as compared to that of the composition prior to purification or treatment to remove the impurity or byproduct. In some embodiments, the level of at least one product-related substance is reduced by at least 30% (w/w), at least 40% (w/w), at least 50% (w/w), at least 60% (w/w), at least 70% (w/w), at least 80% (w/w), at least 90% (w/w), or at least 95% (w/w) as compared to that of a composition prior to purification or treatment to remove the impurity or byproduct. In some embodiments, a circular polyribonucleotide preparation (e.g., a circular polyribonucleotide pharmaceutical preparation or composition or an intermediate in the production of the circular polyribonucleotide preparation) is further substantially free of a process-related impurity or byproduct. In some embodiments, the process-related impurity or byproduct includes a protein (e.g., a cell protein, such as a host cell protein), a deoxyribonucleic acid (e.g., a cell deoxyribonucleic acid, such as a host cell deoxyribonucleic acid), mono deoxyribonucleotide or dideoxyribonucleotide molecules, an enzyme (e.g., a nuclease, such as an endonuclease or exonuclease, or ligase), a reagent component, a gel component, or a chromatographic material. In some embodiments, the impurity or byproduct is selected from: a buffer reagent, a ligase, a nuclease, RNase inhibitor, RNase R, deoxyribonucleotide molecules, acrylamide gel debris, and mono deoxyribonucleotide molecules. In some embodiments, the pharmaceutical preparation includes protein (e.g., cell protein, such as a host cell protein) contamination of less than 0.1 ng, 1 ng, 5 ng, 10 ng, 15 ng, 20 ng, 25 ng, 30 ng, 35 ng, 40 ng, 50 ng, 60 ng, 70 ng, 80 ng, 90 ng, 100 ng, 200 ng, 300 ng, 400 ng, or 500 ng of protein contamination per milligram (mg) of the circular polyribonucleotide molecules.

In an embodiment, a circular polyribonucleotide preparation (e.g., a circular polyribonucleotide pharmaceutical preparation or composition or an intermediate in the production of the circular polyribonucleotide preparation) is substantially free of DNA content e.g., template DNA or cell DNA (e.g., host cell DNA), has a DNA content, as low as zero, or has a DNA content of no more than 1 μg/ml, 10 μg/ml, 0.1 ng/ml, 1 ng/ml, 5 ng/ml, 10 ng/ml, 15 ng/ml, 20 ng/ml, 25 ng/ml, 30 ng/ml, 35 ng/ml, 40 ng/ml, 50 ng/ml, 60 ng/ml, 70 ng/ml, 80 ng/ml, 90 ng/ml, 100 ng/ml, 200 ng/ml, 300 ng/ml, 400 ng/ml, 500 ng/ml, 1000 μg/mL, 5000 μg/mL, 10,000 μg/mL, or 100,000 μg/mL.

In an embodiment, a circular polyribonucleotide preparation (e.g., a circular polyribonucleotide pharmaceutical preparation or composition or an intermediate in the production of the circular polyribonucleotide preparation) is substantially free of DNA content, has a DNA content as low as zero, or has DNA content no more than 0.001% (w/w), 0.01% (w/w), 0.1% (w/w), 1% (w/w), 2% (w/w), 3% (w/w), 4% (w/w), 5% (w/w), 6% (w/w), 7% (w/w), 8% (w/w), 9% (w/w), 10% (w/w), 15% (w/w), 20% (w/w), 25% (w/w), 30% (w/w), 35% (w/w), 40% (w/w), 45% (w/w), 50% (w/w) of total nucleotides on a mass basis, wherein total nucleotide molecules is the total mass of deoxyribonucleotide content and ribonucleotide molecules. In an embodiment, a circular polyribonucleotide preparation (e.g., a circular polyribonucleotide pharmaceutical preparation or composition or an intermediate in the production of the circular polyribonucleotide preparation) is substantially free of DNA content, has DNA content as low as zero, or has DNA content no more than 0.001% (w/w), 0.01% (w/w), 0.1% (w/w), 1% (w/w), 2% (w/w), 3% (w/w), 4% (w/w), 5% (w/w), 6% (w/w), 7% (w/w), 8% (w/w), 9% (w/w), 10% (w/w), 15% (w/w), 20% (w/w), 25% (w/w), 30% (w/w), 35% (w/w), 40% (w/w), 45% (w/w), 50% (w/w) of total nucleotides on a mass basis as measured after a total DNA digestion by enzymes that digest nucleosides by quantitative liquid chromatography-mass spectrometry (LC-MS), in which the content of DNA is back calculated from a standard curve of each base (i.e., A, C, G, T) as measured by LC-MS.

In an embodiment, a circular polyribonucleotide preparation (e.g., a circular polyribonucleotide pharmaceutical preparation or composition or an intermediate in the production of the circular polyribonucleotide preparation) has a protein (e.g., cell protein (CP), e.g., enzyme, a production-related protein, e.g., carrier protein) contamination, impurities, or by-products of no more than 0.1 ng/ml, 1 ng/ml, 5 ng/ml, 10 ng/ml, 15 ng/ml, 20 ng/ml, 25 ng/ml, 30 ng/ml, 35 ng/ml, 40 ng/ml, 50 ng/ml, 60 ng/ml, 70 ng/ml, 80 ng/ml, 90 ng/ml, 100 ng/ml, 200 ng/ml, 300 ng/ml, 400 ng/ml, or 500 ng/ml. In an embodiment, a circular polyribonucleotide (e.g., a circular polyribonucleotide pharmaceutical preparation or composition or an intermediate in the production of the circular polyribonucleotide) has a protein (e.g., production-related protein such as a cell protein (CP), e.g., enzyme) contamination, impurities, or by-products from the limit of detection of up to 0.1 ng/ml, 1 ng/ml, 5 ng/ml, 10 ng/ml, 15 ng/ml, 20 ng/ml, 25 ng/ml, 30 ng/ml, 35 ng/ml, 40 ng/ml, 50 ng/ml, 60 ng/ml, 70 ng/ml, 80 ng/ml, 90 ng/ml, 100 ng/ml, 200 ng/ml, 300 ng/ml, 400 ng/ml, or 500 ng/ml.

In an embodiment, a circular polyribonucleotide preparation (e.g., a circular polyribonucleotide pharmaceutical preparation or composition or an intermediate in the production of the circular polyribonucleotide preparation) has a protein (e.g., production-related protein such as a cell protein (CP), e.g., enzyme) contamination, impurities, or by-products of less than 0.1 ng, 1 ng, 5 ng, 10 ng, 15 ng, 20 ng, 25 ng, 30 ng, 35 ng, 40 ng, 50 ng, 60 ng, 70 ng, 80 ng, 90 ng, 100 ng, 200 ng, 300 ng, 400 ng, or 500 ng per milligram (mg) of the circular polyribonucleotide. In an embodiment, a circular polyribonucleotide (e.g., a circular polyribonucleotide pharmaceutical preparation or composition or an intermediate in the production of the circular polyribonucleotide) has a protein (e.g., production-related protein such as a cell protein (CP), e.g., enzyme) contamination, impurities, or by-products from the level of detection up to 0.1 ng, 1 ng, 5 ng, 10 ng, 15 ng, 20 ng, 25 ng, 30 ng, 35 ng, 40 ng, 50 ng, 60 ng, 70 ng, 80 ng, 90 ng, 100 ng, 200 ng, 300 ng, 400 ng, or 500 ng per milligram (mg) of the circular polyribonucleotide.

In an embodiment, a circular polyribonucleotide preparation (e.g., a circular polyribonucleotide pharmaceutical preparation or composition or an intermediate in the production of the circular polyribonucleotide preparation) has low levels or is substantially absent of endotoxins, e.g., as measured by the Limulus amebocyte lysate (LAL) test. In some embodiments, the pharmaceutical preparation or compositions or an intermediate in the production of the circular polyribonucleotides includes less than 20 EU/kg (weight), 10 EU/kg, 5 EU/kg, 1 EU/kg endotoxin, or lacks endotoxin as measured by the Limulus amebocyte lysate test. In an embodiment, a circular polyribonucleotide composition has low levels or absence of a nuclease or a ligase.

In some embodiments, a circular polyribonucleotide preparation (e.g., a circular polyribonucleotide pharmaceutical preparation or composition or an intermediate in the production of the circular polyribonucleotide preparation) includes no greater than about 50% (w/w), 45% (w/w), 40% (w/w), 35% (w/w), 30% (w/w), 25% (w/w), 20% (w/w), 19% (w/w), 18% (w/w), 17% (w/w), 16% (w/w), 15% (w/w), 14% (w/w), 13% (w/w), 12% (w/w), 11% (w/w), 10% (w/w), 9% (w/w), 8% (w/w), 7% (w/w), 6% (w/w), 5% (w/w), 4% (w/w), 3% (w/w), 2% (w/w), 1% (w/w) of at least one enzyme, e.g., polymerase, e.g., RNA polymerase.

In an embodiment, a circular polyribonucleotide preparation (e.g., a circular polyribonucleotide pharmaceutical preparation or composition or an intermediate in the production of the circular polyribonucleotide preparation) is sterile or substantially free of microorganisms, e.g., the composition or preparation supports the growth of fewer than 100 viable microorganisms as tested under aseptic conditions, the composition or preparation meets the standard of USP <71>, and/or the composition or preparation meets the standard of USP <85>. In some embodiments, the pharmaceutical preparation includes a bioburden of less than 100 CFU/100 ml, 50 CFU/100 ml, 40 CFU/100 ml, 30 CFU/100 ml, 200 CFU/100 ml, 10 CFU/100 ml, or 10 CFU/100 ml before sterilization.

In some embodiments, the circular polyribonucleotide preparation can be further purified using known techniques in the art for removing impurities or byproduct, such as column chromatography or pH/vial inactivation.

In some embodiments, a total weight of polyribonucleotides in the composition includes at least 500 μg (e.g., at least 600 μg, 700 μg, 800 μg, 900 μg, 1 mg, 2 mg, 3 mg, 4 mg, 5 mg, 6 mg, 7 mg, 8 mg, 9 mg, 10 mg, 20 mg, 30 mg, 40 mg, 50 mg, 60 mg, 70 mg, 80 mg, 90 mg, 100 mg, 200 mg, 300 mg, 400 mg, 500 mg, 600 mg, 700 mg, 800 mg, 900 mg, 1,000 mg, or more). In some embodiments, the total weight of polyribonucleotides in the population of polyribonucleotides is from 500 μg to 1000 mg.

### Polynucleotides

The present invention features polyribonucleotides that are used in methods of separation and/or purification and present in compositions described herein. The polyribonucleotides described herein may be linear polyribonucleotides, circular polyribonucleotides or a combination thereof. In some embodiments, a circular polyribonucleotide is produced from a linear polyribonucleotide (e.g., by splicing compatible ends of the linear polyribonucleotide). In some embodiments, a linear polyribonucleotide is transcribed from a deoxyribonucleotide template (e.g., a vector, a linearized vector, or a cDNA). Accordingly, the invention features linear deoxyribonucleotides, circular deoxyribonucleotides, linear polyribonucleotides, and circular polyribonucleotides and compositions thereof useful in the production of polyribonucleotides.

### Linear Polyribonucleotides

The present invention features linear polyribonucleotides that may include one or more of the following: a 3′ intron fragment; a 3′ splice site; a 3′ exon; a polyribonucleotide cargo; a 5′ exon; a 5′ splice site; and a 5′ intron fragment. In some embodiments, the 3′ intron fragment corresponds to a 3′ portion of a catalytic Group I intron, for example, a catalytic Group I intron from a cyanobacterium Anabaena pre-tRNA-Leu gene, a Tetrahymena pre-rRNA, a T4 phage td gene, or a variant thereof. In some embodiments, the 5′ intron fragment corresponds to a 5′ portion of a catalytic Group I intron, for example, a catalytic Group I intron from a cyanobacterium Anabaena pre-tRNA-Leu gene, a Tetrahymena pre-rRNA, a T4 phage td gene, or a variant thereof.

The linear polyribonucleotide may include additional elements, e.g., outside of or between any of elements described above. For example, any of the above elements may be separated by a spacer sequence, as described herein. A target region as described herein may be present in any region of the linear polyribonucleotide as described herein. In some embodiments, the target region is present within an intron or portion thereof.

In certain embodiments, provided herein is a method of generating a linear polyribonucleotide by performing transcription in a cell-free system (e.g., in vitro transcription) using a deoxyribonucleotide (e.g., a vector, linearized vector, or cDNA) provided herein as a template (e.g., a vector, linearized vector, or cDNA provided herein with an RNA polymerase promoter positioned upstream of the region that codes for the linear polyribonucleotide).

A deoxyribonucleotide template may be transcribed to a produce a linear polyribonucleotide containing the components described herein. Upon expression, the linear polyribonucleotide may produce a splicing-compatible polyribonucleotide, which may be spliced in order to produce a circular polyribonucleotide, e.g., for subsequent use.

In some embodiments, the linear polyribonucleotide is from 50 to 20,000, e.g., 300 to 20,000 (e.g., 50, 60, 70, 80, 90, 100, 150, 200, 250, 300, 400, 500, 600, 700, 800, 900, 1,000, 1,100, 1,200, 1,300, 1,400, 1,500, 1,600, 1,700, 1,800, 1,900, 2,000, 2,500, 3,000, 3,500, 4,000, 5,000, 6,000, 7,000, 8,000, 9,000, 10,000, 11,000, 12,000, 13,000, 14,000, 15,000, 16,000, 17,000, 18,000, 19,000, or 20,000) ribonucleotides in length. The linear polyribonucleotide may be, e.g., at least 500, at least 1,000, at least 2,000, at least 3,000, at least 4,000, or at least 5,000 ribonucleotides in length.

### Circular Polyribonucleotides

In some embodiments, the invention features a circular polyribonucleotide. The circular polyribonucleotide may include a splice junction joining a 5′ exon and a 3′ exon. A target region as described herein may be present in any region of the circular polyribonucleotide as described herein. The circular polyribonucleotide may lack in an intron, e.g., after splicing.

The circular polynucleotide may further include a polyribonucleotide cargo. The polyribonucleotide cargo may include an expression sequence, a non-coding sequence, or a combination of an expression sequence and a non-coding sequence. The polyribonucleotide cargo may include an expression sequence encoding a polypeptide. The polyribonucleotide may include an IRES operably linked to an expression sequence encoding a polypeptide. In some embodiments, the circular polyribonucleotide further includes a spacer region between the IRES and the 5′ exon fragment or the 3′ exon fragment. The spacer region may be, e.g., at least 5 (e.g., at least 10, at least 15, at least 20) ribonucleotides in length ribonucleotides in length. The spacer region may be, e.g., from 5 to 500 (e.g., 10, 20, 30, 40, 50, 60, 70, 80, 90, 100, 150, 200, 250, 300, 350, 400, 450, or 500) ribonucleotides. In some embodiments, the spacer region includes a polyA sequence. In some embodiments, the spacer region includes a polyA-C, polyA-G, polyA-U, or other heterogenous or random sequence.

In some embodiments, the circular polyribonucleotide is at least about 20 nucleotides, at least about 30 nucleotides, at least about 40 nucleotides, at least about 50 nucleotides, at least about 75 nucleotides, at least about 100 nucleotides, at least about 200 nucleotides, at least about 300 nucleotides, at least about 400 nucleotides, at least about 500 nucleotides, at least about 1,000 nucleotides, at least about 2,000 nucleotides, at least about 5,000 nucleotides, at least about 6,000 nucleotides, at least about 7,000 nucleotides, at least about 8,000 nucleotides, at least about 9,000 nucleotides, at least about 10,000 nucleotides, at least about 12,000 nucleotides, at least about 14,000 nucleotides, at least about 15,000 nucleotides, at least about 16,000 nucleotides, at least about 17,000 nucleotides, at least about 18,000 nucleotides, at least about 19,000 nucleotides, or at least about 20,000 nucleotides.

In some embodiments, the circular polyribonucleotide may be of a sufficient size to accommodate a binding site for a ribosome. In some embodiments, the size of a circular polyribonucleotide is a length sufficient to encode useful polypeptides, and thus, lengths of at least 20,000 nucleotides, at least 15,000 nucleotides, at least 10,000 nucleotides, at least 7,500 nucleotides, or at least 5,000 nucleotides, at least 4,000 nucleotides, at least 3,000 nucleotides, at least 2,000 nucleotides, at least 1,000 nucleotides, at least 500 nucleotides, at least 1400 nucleotides, at least 300 nucleotides, at least 200 nucleotides, at least 100 nucleotides may be produced.

In some embodiments, the circular polyribonucleotide includes one or more elements described herein. In some embodiments, the elements may be separated from one another by a spacer sequence. In some embodiments, the elements may be separated from one another by 1 ribonucleotide, 2 nucleotides, about 5 nucleotides, about 10 nucleotides, about 15 nucleotides, about 20 nucleotides, about 30 nucleotides, about 40 nucleotides, about 50 nucleotides, about 60 nucleotides, about 80 nucleotides, about 100 nucleotides, about 150 nucleotides, about 200 nucleotides, about 250 nucleotides, about 300 nucleotides, about 400 nucleotides, about 500 nucleotides, about 600 nucleotides, about 700 nucleotides, about 800 nucleotides, about 900 nucleotides, about 1000 nucleotides, up to about 1 kb, at least about 1000 nucleotides, or any amount of nucleotides therebetween. In some embodiments, one or more elements are contiguous with one another, e.g., lacking a spacer element.

In some embodiments, the circular polyribonucleotide may include one or more repetitive elements. In some embodiments, the circular polyribonucleotide includes one or more modifications described herein. In one embodiment, the circular polyribonucleotide contains at least one nucleoside modification. In one embodiment, up to 100% of the nucleosides of the circular polyribonucleotide are modified. In one embodiment, at least one nucleoside modification is a uridine modification or an adenosine modification.

As a result of its circularization, the circular polyribonucleotide may include certain characteristics that distinguish it from a linear polyribonucleotide. For example, the circular polyribonucleotide may contain a target region that is more or less accessible than a linear polyribonucleotide. In some embodiments, the circular polyribonucleotide may be less susceptible to degradation by exonuclease as compared to a linear polyribonucleotide. As such, the circular polyribonucleotide may be more stable than a linear polyribonucleotide, especially when incubated in the presence of an exonuclease. The increased stability of the circular polyribonucleotide compared with a linear polyribonucleotide makes circular polyribonucleotide more useful as a cell transforming reagent to produce polypeptides and can be stored more easily and for longer than a linear polyribonucleotide. The stability of the circular polyribonucleotide treated with exonuclease can be tested using methods standard in art which determine whether RNA degradation has occurred (e.g., by gel electrophoresis). Moreover, unlike a linear polyribonucleotide, the circular polyribonucleotide may be less susceptible to dephosphorylation when the circular polyribonucleotide is incubated with phosphatase, such as calf intestine phosphatase.

### Polyribonucleotide Cargo

A polyribonucleotide cargo described herein includes any sequence including at least one polyribonucleotide. In some embodiments, the polyribonucleotide cargo includes an expression sequence, a non-coding sequence, or an expression sequence and a non-coding sequence. In some embodiments, the polyribonucleotide cargo includes an expression sequence encoding a polypeptide. In some embodiments, the polyribonucleotide cargo includes an IRES operably linked to an expression sequence encoding a polypeptide. In some embodiments, the polyribonucleotide cargo includes an expression sequence that encodes a polypeptide that has a biological effect on a subject.

A polyribonucleotide cargo may, for example, include at least about 40 nucleotides, at least about 50 nucleotides, at least about 75 nucleotides, at least about 100 nucleotides, at least about 200 nucleotides, at least about 300 nucleotides, at least about 400 nucleotides, at least about 500 nucleotides, at least about 1,000 nucleotides, at least about 2,000 nucleotides, at least about 5,000 nucleotides, at least about 6,000 nucleotides, at least about 7,000 nucleotides, at least about 8,000 nucleotides, at least about 9,000 nucleotides, at least about 10,000 nucleotides, at least about 12,000 nucleotides, at least about 14,000 nucleotides, at least about 15,000 nucleotides, at least about 16,000 nucleotides, at least about 17,000 nucleotides, at least about 18,000 nucleotides, at least about 19,000 nucleotides, or at least about 20,000 nucleotides. In some embodiments, the polyribonucleotides cargo includes from 1-20,000 nucleotides, 1-10,000 nucleotides, 1-5,000 nucleotides, 100-20,000 nucleotide, 100-10,000 nucleotides, 100-5,000 nucleotides, 500-20,000 nucleotides, 500-10,000 nucleotides, 500-5,000 nucleotides, 1,000-20,000 nucleotides, 1,000-10,000 nucleotides, or 1,000-5,000 nucleotides.

In embodiments, the polyribonucleotide cargo includes one or multiple expression (or coding) sequences, wherein each expression (or coding) sequence encodes a polypeptide. In embodiments, the polyribonucleotide cargo includes ones or multiple noncoding sequences. In embodiments, the polyribonucleotide cargo consists entirely of non-coding sequence(s). In embodiments, the polyribonucleotide cargo includes a combination of expression (or coding) and noncoding sequences.

In some embodiments, polyribonucleotides made as described herein are used as effectors in therapy or agriculture. For example, a circular polyribonucleotide made by the methods described herein (e.g., the cell-free methods described herein) may be administered to a subject (e.g., in a pharmaceutical, veterinary, or agricultural composition). In another example, a circular polyribonucleotide made by the methods described herein (e.g., the cell-free methods described herein) may be delivered to a cell.

In some embodiments, the polyribonucleotide includes any feature, or any combination of features as disclosed in PCT Publication No. WO2019/118919, which is hereby incorporated by reference in its entirety.

In some embodiments, the polyribonucleotide cargo includes an open reading frame. In some embodiments, the open reading frame is operably linked to an IRES. The open reading frame may encode an RNA or a polypeptide. In some embodiments, the open reading frame encodes a polypeptide and the polyribonucleotide (e.g., circular polyribonucleotide) provides increased expression (e.g., by at least 5%, 10%, 15%, 20%, 25%, 30%, 40%, 50%, 60%, 70%, 80%, 90%, 100%, or more) of the polypeptide, e.g., as compared to a linear polyribonucleotide encoding the polypeptide. In some embodiments, increased purity of the polyribonucleotide, e.g., a circular polyribonucleotide, results in increased expression (e.g., by at least 5%, 10%, 15%, 20%, 25%, 30%, 40%, 50%, 60%, 70%, 80%, 90%, 100%, or more) of the polypeptide, e.g., as compared to a population of circular and linear polyribonucleotides.

### Polypeptide Expression Sequences

In some embodiments, the polyribonucleotide described herein (e.g., the polyribonucleotide cargo of a circular polyribonucleotide) includes one or more expression (or coding) sequences, wherein each expression sequence encodes a polypeptide. In some embodiments, the circular polyribonucleotide includes two, three, four, five, six, seven, eight, nine, ten or more expression (or coding) sequences.

Each encoded polypeptide may be linear or branched. In embodiments, the polypeptide has a length from about 5 to about 40,000 amino acids, about 15 to about 35,000 amino acids, about 20 to about 30,000 amino acids, about 25 to about 25,000 amino acids, about 50 to about 20,000 amino acids, about 100 to about 15,000 amino acids, about 200 to about 10,000 amino acids, about 500 to about 5,000 amino acids, about 1,000 to about 2,500 amino acids, or any range therebetween. In some embodiments, the polypeptide has a length of less than about 40,000 amino acids, less than about 35,000 amino acids, less than about 30,000 amino acids, less than about 25,000 amino acids, less than about 20,000 amino acids, less than about 15,000 amino acids, less than about 10,000 amino acids, less than about 9,000 amino acids, less than about 8,000 amino acids, less than about 7,000 amino acids, less than about 6,000 amino acids, less than about 5,000 amino acids, less than about 4,000 amino acids, less than about 3,000 amino acids, less than about 2,500 amino acids, less than about 2,000 amino acids, less than about 1,500 amino acids, less than about 1,000 amino acids, less than about 900 amino acids, less than about 800 amino acids, less than about 700 amino acids, less than about 600 amino acids, less than about 500 amino acids, less than about 400 amino acids, less than about 300 amino acids, or less may be useful.

Polypeptides included herein may include naturally occurring polypeptides or non-naturally occurring polypeptides. In some embodiments, the polypeptide is or includes a functional fragment or variant of a reference polypeptide (e.g., an enzymatically active fragment or variant of an enzyme). For example, the polypeptide may be a functionally active variant of any of the polypeptides described herein with at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identity, e.g., over a specified region or over the entire sequence, to a sequence of a polypeptide described herein or a naturally occurring polypeptide. In some instances, the polypeptide may have at least 50% (e.g., at least 50%, 60%, 70%, 80%, 90%, 95%, 97%, 99%, or greater) identity to a protein of interest.

Some examples of a polypeptide include, but are not limited to, a fluorescent tag or marker, an antigen, a therapeutic polypeptide, a plant-modifying polypeptide, or a polypeptide for agricultural applications.

A therapeutic polypeptide may be a hormone, a neurotransmitter, a growth factor, an enzyme (e.g., oxidoreductase, metabolic enzyme, mitochondrial enzyme, oxygenase, dehydrogenase, ATP-independent enzyme, lysosomal enzyme, desaturase), a cytokine, an antigen binding polypeptide (e.g., antigen binding antibody or antibody-like fragments, such as single chain antibodies, nanobodies or other Ig heavy chain or light chain containing polypeptides), an Fc fusion protein, an anticoagulant, a blood factor, a bone morphogenetic protein, an interferon, an interleukin, and a thrombolytic.

A polypeptide for agricultural applications may be a bacteriocin, a lysin, an antimicrobial polypeptide, an antifungal polypeptide, a nodule C-rich peptide, a bacteriocyte regulatory peptide, a peptide toxin, a pesticidal polypeptide (e.g., insecticidal polypeptide or nematocidal polypeptide), an antigen binding polypeptide (e.g., antigen binding antibody or antibody-like fragments, such as single chain antibodies, nanobodies or other Ig heavy chain or light chain containing polypeptides), an enzyme (e.g., nuclease, amylase, cellulase, peptidase, lipase, chitinase), a peptide pheromone, and a transcription factor.

In some cases, the polyribonucleotide expresses a non-human protein.

In some embodiments, the polyribonucleotide expresses an antibody, e.g., an antibody fragment, or a portion thereof. In some embodiments, the antibody expressed by the circular polyribonucleotide can be of any isotype, such as IgA, IgD, IgE, IgG, IgM. In some embodiments, the circular polyribonucleotide expresses a portion of an antibody, such as a light chain, a heavy chain, a Fc fragment, a CDR (complementary determining region), a Fv fragment, or a Fab fragment, a further portion thereof. In some embodiments, the circular polyribonucleotide expresses one or more portions of an antibody. For instance, the circular polyribonucleotide can include more than one expression sequence, each of which expresses a portion of an antibody, and the sum of which can constitute the antibody. In some cases, the circular polyribonucleotide includes one expression sequence coding for the heavy chain of an antibody, and another expression sequence coding for the light chain of the antibody. In some cases, when the circular polyribonucleotide is expressed in a cell or a cell-free environment, the light chain and heavy chain can be subject to appropriate modification, folding, or other post-translation modification to form a functional antibody.

In embodiments, polypeptides include multiple polypeptides, e.g., multiple copies of one polypeptide sequence, or multiple different polypeptide sequences. In embodiments, multiple polypeptides are connected by linker amino acids or spacer amino acids.

In embodiments, the polynucleotide cargo includes a sequence encoding a signal peptide. Many signal peptide sequences have been described, for example, the Tat (Twin-arginine translocation) signal sequence is typically an N-terminal peptide sequence containing a consensus SRRxFLK “twin-arginine” motif, which serves to translocate a folded protein containing such a Tat signal peptide across a lipid bilayer. See also, e.g., the Signal Peptide Database publicly available at www[dot]signalpeptide[dot]de. Signal peptides are also useful for directing a protein to specific organelles; see, e.g., the experimentally determined and computationally predicted signal peptides disclosed in the Spdb signal peptide database, publicly available at proline.bic.nus.edu.sg/spdb.

In embodiments, the polynucleotide cargo includes sequence encoding a cell-penetrating peptide (CPP). Hundreds of CPP sequences have been described; see, e.g., the database of cell-penetrating peptides, CPPsite, publicly available at crdd.osdd.net/raghava/cppsite/. An example of a commonly used CPP sequence is a poly-arginine sequence, e.g., octoarginine or nonoarginine, which can be fused to the C-terminus of the CGI peptide.

In embodiments, the polynucleotide cargo includes sequence encoding a self-assembling peptide; see, e.g., Miki et al. (2021) Nature Communications, 21:3412, DOI: 10.1038/s41467-021-23794-6.

In some embodiments, the expression sequence includes a poly-A sequence (e.g., at the 3′ end of an expression sequence). In some embodiments, the length of a poly-A sequence is greater than 10 nucleotides in length. In one embodiment, the poly-A sequence is greater than 15 nucleotides in length (e.g., at least or greater than about 10, 15, 20, 25, 30, 35, 40, 45, 50, 55, 60, 70, 80, 90, 100, 120, 140, 160, 180, 200, 250, 300, 350, 400, 450, 500, 600, 700, 800, 900, 1,000, 1,100, 1,200, 1,300, 1,400, 1,500, 1,600, 1,700, 1,800, 1,900, 2,000, 2,500, and 3,000 nucleotides). In some embodiments, the poly-A sequence is designed according to the descriptions of the poly-A sequence in [0202]-[0204] of International Patent Publication No. WO2019/118919A1, which is incorporated herein by reference in its entirety. In some embodiments, the expression sequence lacks a poly-A sequence (e.g., at the 3′ end of an expression sequence).

In some embodiments, a circular polyribonucleotide includes a polyA, lacks a polyA, or has a modified polyA to modulate one or more characteristics of the circular polyribonucleotide. In some embodiments, the circular polyribonucleotide lacking a polyA or having modified polyA improves one or more functional characteristics, e.g., immunogenicity (e.g., the level of one or more marker of an immune or inflammatory response), half-life, and/or expression efficiency.

### Therapeutic Polypeptides

In some embodiments, a polyribonucleotide described herein (e.g., the polyribonucleotide cargo of the circular polyribonucleotide) includes at least one expression sequence encoding a therapeutic polypeptide. A therapeutic polypeptide is a polypeptide that when administered to or expressed in a subject provides some therapeutic benefit. Administration to a subject or expression in a subject of a therapeutic polypeptide may be used to treat or prevent a disease, disorder, or condition or a symptom thereof. In some embodiments, the circular polyribonucleotide encodes two, three, four, five, six, seven, eight, nine, ten or more therapeutic polypeptides.

In some embodiments, the polyribonucleotide includes an expression sequence encoding a therapeutic protein. The protein may treat the disease in the subject in need thereof. In some embodiments, the therapeutic protein can compensate for a mutated, under-expressed, or absent protein in the subject in need thereof. In some embodiments, the therapeutic protein can target, interact with, or bind to a cell, tissue, or virus in the subject in need thereof.

A therapeutic polypeptide can be a polypeptide that can be secreted from a cell, or localized to the cytoplasm, nucleus, or membrane compartment of a cell.

A therapeutic polypeptide may be a hormone, a neurotransmitter, a growth factor, an enzyme (e.g., oxidoreductase, metabolic enzyme, mitochondrial enzyme, oxygenase, dehydrogenase, ATP-independent enzyme, lysosomal enzyme, desaturase), a cytokine, a transcription factor, an antigen binding polypeptide (e.g., antigen binding antibody or antibody-like fragments, such as single chain antibodies, nanobodies or other Ig heavy chain or light chain containing polypeptides), an Fc fusion protein, an anticoagulant, a blood factor, a bone morphogenetic protein, an interferon, an interleukin, a thrombolytic, an antigen (e.g., a tumor, viral, or bacterial antigen), a nuclease (e.g., an endonuclease such as a Cas protein, e.g., Cas9), a membrane protein (e.g., a chimeric antigen receptor (CAR), a transmembrane receptor, a G-protein-coupled receptor (GPCR), a receptor tyrosine kinase (RTK), an antigen receptor, an ion channel, or a membrane transporter), a secreted protein, a gene editing protein (e.g., a CRISPR-Cas, TALEN, or zinc finger), or a gene writing protein (see, e.g., International Patent Publication No. WO2020/047124, incorporated in its entirety herein by reference).

In some embodiments, the therapeutic polypeptide is an antibody, e.g., a full-length antibody, an antibody fragment, or a portion thereof. In some embodiments, the antibody expressed by the polyribonucleotide (e.g., circular polyribonucleotide) can be of any isotype, such as IgA, IgD, IgE, IgG, IgM. In some embodiments, the polyribonucleotide expresses a portion of an antibody, such as a light chain, a heavy chain, a Fc fragment, a CDR (complementary determining region), a Fv fragment, or a Fab fragment, a further portion thereof. In some embodiments, the polyribonucleotide expresses one or more portions of an antibody. For instance, the polyribonucleotide can include more than one expression sequence, each of which expresses a portion of an antibody, and the sum of which can constitute the antibody. In some cases, the polyribonucleotide includes one expression sequence coding for the heavy chain of an antibody, and another expression sequence coding for the light chain of the antibody. When the polyribonucleotide is expressed in a cell, the light chain and heavy chain can be subject to appropriate modification, folding, or other post-translation modification to form a functional antibody.

In some embodiments, polyribonucleotides made as described herein (e.g., circular polyribonucleotides) are used as effectors in therapy or agriculture. For example, a polyribonucleotide made by the methods described herein may be administered to a subject (e.g., in a pharmaceutical, veterinary, or agricultural composition). In embodiments, the subject is a vertebrate animal (e.g., mammal, bird, fish, reptile, or amphibian). In embodiments, the subject is a human. In embodiments, the method subject is a non-human mammal. In embodiments, the subject is a non-human mammal such as a non-human primate (e.g., monkeys, apes), ungulate (e.g., cattle, buffalo, sheep, goat, pig, camel, llama, alpaca, deer, horses, donkeys), carnivore (e.g., dog, cat), rodent (e.g., rat, mouse), or lagomorph (e.g., rabbit). In embodiments, the subject is a bird, such as a member of the avian taxa Galliformes (e.g., chickens, turkeys, pheasants, quail), Anseriformes (e.g., ducks, geese), Paleaognathae (e.g., ostriches, emus), Columbiformes (e.g., pigeons, doves), or Psittaciformes (e.g., parrots). In embodiments, the subject is an invertebrate such as an arthropod (e.g., insects, arachnids, crustaceans), a nematode, an annelid, a helminth, or a mollusc. In embodiments, the subject is an invertebrate agricultural pest or an invertebrate that is parasitic on an invertebrate or vertebrate host. In embodiments, the subject is a plant, such as an angiosperm plant (which can be a dicot or a monocot) or a gymnosperm plant (e.g., a conifer, a cycad, a gnetophyte, a Ginkgo), a fern, horsetail, clubmoss, or a bryophyte. In embodiments, the subject is a eukaryotic alga (unicellular or multicellular). In embodiments, the subject is a plant of agricultural or horticultural importance, such as row crop plants, fruit-producing plants and trees, vegetables, trees, and ornamental plants including ornamental flowers, shrubs, trees, groundcovers, and turf grasses.

### Plant-Modifying Polypeptides

In some embodiments, the polyribonucleotide described herein (e.g., the polyribonucleotide cargo of the polyribonucleotide) includes at least one expression sequence encoding a plant-modifying polypeptide. A plant-modifying polypeptide refers to a polypeptide that can alter the genetic properties (e.g., increase gene expression, decrease gene expression, or otherwise alter the nucleotide sequence of DNA or RNA), epigenetic properties, or physiological or biochemical properties of a plant in a manner that results in change in the plant's physiology or phenotype, e.g., an increase or decrease in the plant's fitness. In some embodiments, the polyribonucleotide encodes two, three, four, five, six, seven, eight, nine, ten or more different plant-modifying polypeptides, or multiple copies of one or more plant-modifying polypeptides. A plant-modifying polypeptide may change the physiology or phenotype of or increase or decrease the fitness of a variety of plants or can be one that effects such change(s) in one or more specific plants (e.g., a specific species or genera of plants).

Examples of polypeptides that can be used herein can include an enzyme (e.g., a metabolic recombinase, a helicase, an integrase, a RNAse, a DNAse, or a ubiquitination protein), a pore-forming protein, a signaling ligand, a cell penetrating peptide, a transcription factor, a receptor, an antibody, a nanobody, a gene editing protein (e.g., CRISPR-Cas endonuclease, TALEN, or zinc finger), riboprotein, a protein aptamer, or a chaperone.

### Agricultural Polypeptides

In some embodiments, the polyribonucleotide described herein (e.g., the polyribonucleotide cargo of the polyribonucleotide) includes at least one expression sequence encoding an agricultural polypeptide. An agricultural polypeptide is a polypeptide that is suitable for an agricultural use. In embodiments, an agricultural polypeptide is applied to a plant or seed (e.g., by foliar spray, dusting, injection, or seed coating) or to the plant's environment (e.g., by soil drench or granular soil application), resulting in an alteration of the plant's physiology, phenotype, or fitness. Embodiments of an agricultural polypeptide include polypeptides that alter a level, activity, or metabolism of one or more microorganisms that are resident in or on a plant or non-human animal host, the alteration resulting in an increase in the host's fitness. In some embodiments the agricultural polypeptide is a plant polypeptide. In some embodiments, the agricultural polypeptide is an insect polypeptide. In some embodiments, the agricultural polypeptide has a biological effect when contacted with a non-human vertebrate animal, invertebrate animal, microbial, or plant cell.

In some embodiments, the polyribonucleotide encodes two, three, four, five, six, seven, eight, nine, ten or more agricultural polypeptides, or multiple copies of one or more agricultural polypeptides.

Embodiments of polypeptides useful in agricultural applications include, for example, bacteriocins, lysins, antimicrobial peptides, nodule C-rich peptides, and bacteriocyte regulatory peptides. Such polypeptides can be used to alter the level, activity, or metabolism of target microorganisms for increasing the fitness of insects, such as honeybees and silkworms. Embodiments of agriculturally useful polypeptides include peptide toxins, such as those naturally produced by entomopathogenic bacteria (e.g., Bacillus thuringiensis, Photorhabdus luminescens, Serratia entomophila, or Xenorhabdus nematophila), as is known in the art. Embodiments of agriculturally useful polypeptides include polypeptides (including small peptides such as cyclodipeptides or diketopiperazines) for controlling agriculturally important pests or pathogens, e.g., antimicrobial polypeptides or antifungal polypeptides for controlling diseases in plants, or pesticidal polypeptides (e.g., insecticidal polypeptides or nematicidal polypeptides) for controlling invertebrate pests such as insects or nematodes. Embodiments of agriculturally useful polypeptides include antibodies, nanobodies, and fragments thereof, e.g., antibody or nanobody fragments that retain at least some (e.g., at least 10%) of the specific binding activity of the intact antibody or nanobody. Embodiments of agriculturally useful polypeptides include transcription factors, e.g., plant transcription factors; see, e.g., the “AtTFDB” database listing the transcription factor families identified in the model plant Arabidopsis thaliana), publicly available at agris-knowledgebase[dot]org/AtTFDB/. Embodiments of agriculturally useful polypeptides include nucleases, for example, exonucleases or endonucleases (e.g., Cas nucleases such as Cas9 or Cas12a). Embodiments of agriculturally useful polypeptides further include cell-penetrating peptides, enzymes (e.g., amylases, cellulases, peptidases, lipases, chitinases), peptide pheromones (for example, yeast mating pheromones, invertebrate reproductive and larval signaling pheromones, see, e.g., Altstein (2004) Peptides, 25:1373-1376).

As used herein “increasing fitness” or “promoting fitness” of a subject refers to any favorable alteration in physiology, or of any activity carried out by a subject organism, as a consequence of administration of a peptide or polypeptide described herein, including, but not limited to, any one or more of the following desired effects: (1) increased tolerance of biotic or abiotic stress; (2) increased yield or biomass; (3) modified flowering time; (4) increased resistance to pests or pathogens; (4) increased resistance to herbicides; (5) increasing a population of a subject organism; (6) increasing the reproductive rate of a subject organism; (7) increasing the mobility of a subject organism; (8) increasing the body weight of a subject organism; (9) increasing the metabolic rate or activity of a subject organism; (10) increasing pollination; (11) increasing production of subject organism; (12) increasing nutrient content of the subject organism; (13) increasing a subject organism's resistance to pesticides; or (14) increasing health or reducing disease of a subject organism such as a human or non-human animal. An increase in host fitness can be determined in comparison to a subject organism to which the modulating agent has not been administered. Conversely, “decreasing fitness” of a subject refers to any unfavorable alteration in physiology, or of any activity carried out by a subject organism, as a consequence of administration of a peptide or polypeptide described herein, including, but not limited to, a decrease in any one or more of the effects listed above.

### Internal Ribosomal Entry Site

In some embodiments, a polyribonucleotide described herein includes one or more internal ribosome entry site (IRES) elements. In some embodiments, the IRES is operably linked to one or more expression sequences (e.g., each IRES is operably linked to one or more expression sequences. In embodiments, the IRES is located between a heterologous promoter and the 5′ end of a coding sequence.

A suitable IRES element to include in a polyribonucleotide includes an RNA sequence capable of engaging a eukaryotic ribosome. In some embodiments, the IRES element is at least about 5 nt, at least about 8 nt, at least about 9 nt, at least about 10 nt, at least about 15 nt, at least about 20 nt, at least about 25 nt, at least about 30 nt, at least about 40 nt, at least about 50 nt, at least about 100 nt, at least about 200 nt, at least about 250 nt, at least about 350 nt, or at least about 500 nt.

In some embodiments, the IRES element is derived from the DNA of an organism including, but not limited to, a virus, a mammal, and a Drosophila. Such viral DNA may be derived from, but is not limited to, picornavirus complementary DNA (cDNA), with encephalomyocarditis virus (EMCV) cDNA and poliovirus cDNA. In one embodiment, Drosophila DNA from which an IRES element is derived includes, but is not limited to, an Antennapedia gene from Drosophila melanogaster.

In some embodiments, the IRES sequence is an IRES sequence of Taura syndrome virus, Triatoma virus, Theiler's encephalomyelitis virus, simian Virus 40, Solenopsis invicta virus 1, Rhopalosiphum padi virus, Reticuloendotheliosis virus, fuman poliovirus 1, Plautia stall intestine virus, Kashmir bee virus, Human rhinovirus 2 (HRV-2), Homalodisca coagulata virus-1, Human Immunodeficiency Virus type 1, Homalodisca coagulata virus-1, Himetobi P virus, Hepatitis C virus, Hepatitis A virus, Hepatitis GB virus, foot and mouth disease virus, Human enterovirus 71, Equine rhinitis virus, Ectropis obliqua picorna-like virus, Encephalomyocarditis virus (EMCV), Drosophila C Virus, Crucifer tobamo virus, Cricket paralysis virus, Bovine viral diarrhea virus 1, Black Queen Cell Virus, Aphid lethal paralysis virus, Avian encephalomyelitis virus (AEV), Acute bee paralysis virus, Hibiscus chlorotic ringspot virus, Classical swine fever virus, Human FGF2, Human SFTPA1, Human AML1/RUNX1, Drosophila antennapedia, Human AQP4, Human AT1R, Human BAG-I, Human BCL2, Human BiP, Human c-IAPI, Human c-myc, Human eIF4G, Mouse NDST4L, Human LEF1, Mouse HIF1 alpha, Human n.myc, Mouse Gtx, Human p27kipl, Human PDGF2/c-sis, Human p53, Human Pim-1, Mouse Rbm3, Drosophila reaper, Canine Scamper, Drosophila Ubx, Human UNR, Mouse UtrA, Human VEGF-A, Human XIAP, Salivirus, Cosavirus, Parechovirus, Drosophila hairless, S. cerevisiae TFIID, S. cerevisiae YAP1, Human c-src, Human FGF-1, Simian picomavirus, Turnip crinkle virus, Aichivirus, Crohivirus, Echovirus 11, an aptamer to eIF4G, Coxsackievirus B3 (CVB3) or Coxsackievirus A (CVB1/2). In yet another embodiment, the IRES is an IRES sequence of Coxsackievirus B3 (CVB3). In a further embodiment, the IRES is an IRES sequence of Encephalomyocarditis virus (EMCV). In a further embodiment, the IRES is an IRES sequence of Theiler's encephalomyelitis virus.

In some embodiments, the IRES sequence has a modified sequence in comparison to the wild-type IRES sequence. In some embodiments, when the last nucleotide of the wild-type IRES is not a cytosine nucleic acid residue, the last nucleotide of the wild-type IRES sequence is modified such that it is a cytosine residue. For example, in some embodiments, the IRES sequence is a a CVB3 IRES sequence wherein the terminal adenosine residue is modified to cytosine residue.

In some embodiments, the IRES sequence is an Enterovirus 71 (EV17) IRES, wherein the terminal guanosine residue of the EV17 IRES sequence is modified to a cytosine residue.

In some embodiments, the polyribonucleotide includes at least one IRES flanking at least one (e.g., 2, 3, 4, 5 or more) expression sequence. In some embodiments, the IRES flanks both sides of at least one (e.g., 2, 3, 4, 5 or more) expression sequence. In some embodiments, the polyribonucleotide includes one or more IRES sequences on one or both sides of each expression sequence, leading to separation of the resulting peptide(s) and or polypeptide(s).

In some embodiments, a polyribonucleotide described herein includes an IRES (e.g., an IRES operably linked to a coding region). For example, the polyribonucleotide may include any IRES as described in Chen et al. MOL. CELL 81(20):4300-18, 2021; Jopling et al. ONCOGENE 20:2664-70, 2001; Baranick et al. PNAS 105(12):4733-38, 2008; Lang et al. MOLECULAR BIOLOGY OF THE CELL 13(5):1792-1801, 2002; Dorokhov et al. PNAS 99(8):5301-06, 2002; Wang et al. NUCLEIC ACIDS RESEARCH 33(7):2248-58, 2005; Petz et al. NUCLEIC ACIDS RESEARCH 35(8):2473-82, 2007; Chen et al. SCIENCE 268:415-417, 1995; Fan et al. NATURE COMMUNICATION 13(1):3751-3765, 2022, and International Publication No. WO2021/263124, each of which is hereby incorporated by reference in their entirety.

### Regulatory Elements

In some embodiments, the polyribonucleotide described herein (e.g., the polyribonucleotide cargo of the polyribonucleotide) includes one or more regulatory elements. In some embodiments, the polyribonucleotide includes a regulatory element, e.g., a sequence that modifies expression of an expression sequence within the polyribonucleotide.

A regulatory element may include a sequence that is located adjacent to an expression sequence that encodes an expression product. A regulatory element may be linked operatively to the adjacent sequence. A regulatory element may increase an amount of product expressed as compared to an amount of the expressed product when no regulatory element exists. In addition, one regulatory element can increase the amount or number of products expressed for multiple expression sequences attached in tandem. Hence, one regulatory element can enhance the expression of one or more expression sequences. Multiple regulatory elements can also be used, for example, to differentially regulate expression of different expression sequences.

In some embodiments, the regulatory element is a translation modulator. A translation modulator can modulate translation of the expression sequence in the polyribonucleotide. A translation modulator can be a translation enhancer or suppressor. In some embodiments, the polyribonucleotide includes at least one translation modulator adjacent to at least one expression sequence. In some embodiments, the polyribonucleotide includes a translation modulator adjacent each expression sequence. In some embodiments, the translation modulator is present on one or both sides of each expression sequence, leading to separation of the expression products, e.g., peptides and or polypeptides.

In some embodiments, the regulatory element is a microRNA (miRNA) or a miRNA binding site.

Further examples of regulatory elements are described, e.g., in paragraphs [0154]-[0161] of International Patent Publication No. WO2019/118919, which is hereby incorporated by reference in its entirety.

### Translation Initiation Sequences

In some embodiments, the polyribonucleotide described herein (e.g., the polyribonucleotide cargo of the polyribonucleotide) includes at least one translation initiation sequence. In some embodiments, the polyribonucleotide includes a translation initiation sequence operably linked to an expression sequence.

In some embodiments, the polyribonucleotide encodes a polypeptide and may include a translation initiation sequence, e.g., a start codon. In some embodiments, the translation initiation sequence includes a Kozak or Shine-Dalgamo sequence. In some embodiments, the polyribonucleotide includes the translation initiation sequence, e.g., Kozak sequence, adjacent to an expression sequence. In some embodiments, the translation initiation sequence is a non-coding start codon. In some embodiments, the translation initiation sequence, e.g., Kozak sequence, is present on one or both sides of each expression sequence, leading to separation of the expression products. In some embodiments, the polyribonucleotide includes at least one translation initiation sequence adjacent to an expression sequence. In some embodiments, the translation initiation sequence provides conformational flexibility to the polyribonucleotide. In some embodiments, the translation initiation sequence is within a substantially single stranded region of the polyribonucleotide. Further examples of translation initiation sequences are described in paragraphs [0163]-[0165] of International Patent Publication No. WO2019/118919, which is hereby incorporated by reference in its entirety.

The polyribonucleotide may include more than 1 start codon such as, but not limited to, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 25, at least 30, at least 35, at least 40, at least 50, at least 60 or more than 60 start codons. Translation may initiate on the first start codon or may initiate downstream of the first start codon.

In some embodiments, the polyribonucleotide may initiate at a codon which is not the first start codon, e.g., AUG. Translation of the polyribonucleotide may initiate at an alternative translation initiation sequence, such as, but not limited to, ACG, AGG, AAG, CTG/CUG, GTG/GUG, ATA/AUA, ATT/AUU, TTG/UUG. In some embodiments, translation begins at an alternative translation initiation sequence under selective conditions, e.g., stress induced conditions. As a non-limiting example, the translation of the polyribonucleotide may begin at alternative translation initiation sequence, such as ACG. As another non-limiting example, the polyribonucleotide translation may begin at alternative translation initiation sequence, CTG/CUG. As another non-limiting example, the polyribonucleotide translation may begin at alternative translation initiation sequence, GTG/GUG. As another non-limiting example, the polyribonucleotide may begin translation at a repeat-associated non-AUG (RAN) sequence, such as an alternative translation initiation sequence that includes short stretches of repetitive RNA e.g., CGG, GGGGCC, CAG, CTG.

### Termination Elements

In some embodiments, the polyribonucleotide described herein (e.g., the polyribonucleotide cargo of the polyribonucleotide) includes least one termination element. In some embodiments, the polyribonucleotide includes a termination element operably linked to an expression sequence. In some embodiments, the polynucleotide lacks a termination element.

In some embodiments, the polyribonucleotide includes one or more expression sequences, and each expression sequence may or may not have a termination element. In some embodiments, the polyribonucleotide includes one or more expression sequences, and the expression sequences lack a termination element, such that the polyribonucleotide is continuously translated. Exclusion of a termination element may result in rolling circle translation or continuous expression of expression product.

In some embodiments, the circular polyribonucleotide includes one or more expression sequences, and each expression sequence may or may not have a termination element. In some embodiments, the circular polyribonucleotide includes one or more expression sequences, and the expression sequences lack a termination element, such that the circular polyribonucleotide is continuously translated. Exclusion of a termination element may result in rolling circle translation or continuous expression of expression product, e.g., peptides or polypeptides, due to lack of ribosome stalling or fall-off. In such an embodiment, rolling circle translation expresses a continuous expression product through each expression sequence. In some other embodiments, a termination element of an expression sequence can be part of a stagger element. In some embodiments, one or more expression sequences in the circular polyribonucleotide includes a termination element. However, rolling circle translation or expression of a succeeding (e.g., second, third, fourth, fifth, etc.) expression sequence in the circular polyribonucleotide is performed. In such instances, the expression product may fall off the ribosome when the ribosome encounters the termination element, e.g., a stop codon, and terminates translation. In some embodiments, translation is terminated while the ribosome, e.g., at least one subunit of the ribosome, remains in contact with the circular polyribonucleotide.

In some embodiments, the circular polyribonucleotide includes a termination element at the end of one or more expression sequences. In some embodiments, one or more expression sequences includes two or more termination elements in succession. In such embodiments, translation is terminated and rolling circle translation is terminated. In some embodiments, the ribosome completely disengages with the circular polyribonucleotide. In some such embodiments, production of a succeeding (e.g., second, third, fourth, fifth, etc.) expression sequence in the circular polyribonucleotide may require the ribosome to reengage with the circular polyribonucleotide prior to initiation of translation. Generally, termination elements include an in-frame nucleotide triplet that signals termination of translation, e.g., UAA, UGA, UAG. In some embodiments, one or more termination elements in the circular polyribonucleotide are frame-shifted termination elements, such as but not limited to, off-frame or −1 and +1 shifted reading frames (e.g., hidden stop) that may terminate translation. Frame-shifted termination elements include nucleotide triples, TAA, TAG, and TGA that appear in the second and third reading frames of an expression sequence. Frame-shifted termination elements may be important in preventing misreads of mRNA, which is often detrimental to the cell. In some embodiments, the termination element is a stop codon.

Further examples of termination elements are described in paragraphs [0169]-[0170] of International Patent Publication No. WO2019/118919, which is hereby incorporated by reference in its entirety.

### Untranslated Regions

In some embodiments, a circular polyribonucleotide includes untranslated regions (UTRs). UTRs of a genomic region including a gene may be transcribed but not translated. In some embodiments, a UTR may be included upstream of the translation initiation sequence of an expression sequence described herein. In some embodiments, a UTR may be included downstream of an expression sequence described herein. In some instances, one UTR for a first expression sequence is the same as or continuous with or overlapping with another UTR for a second expression sequence.

Exemplary untranslated regions are described in paragraphs [0197]-[201] of International Patent Publication No. WO2019/118919, which is hereby incorporated by reference in its entirety.

In some embodiments, a circular polyribonucleotide includes a poly-A sequence. Exemplary poly-A sequences are described in paragraphs [0202]-[0205] of International Patent Publication No. WO2019/118919, which is hereby incorporated by reference in its entirety. In some embodiments, a circular polyribonucleotide lacks a poly-A sequence.

In some embodiments, a circular polyribonucleotide includes a UTR with one or more stretches of Adenosines and Uridines embedded within. These AU rich signatures may increase turnover rates of the expression product.

Introduction, removal, or modification of UTR AU rich elements (AREs) may be useful to modulate the stability, or immunogenicity (e.g., the level of one or more marker of an immune or inflammatory response) of the circular polyribonucleotide. When engineering specific circular polyribonucleotides, one or more copies of an ARE may be introduced to the circular polyribonucleotide and the copies of an ARE may modulate translation and/or production of an expression product. Likewise, AREs may be identified and removed or engineered into the circular polyribonucleotide to modulate the intracellular stability and thus affect translation and production of the resultant protein.

It should be understood that any UTR from any gene may be incorporated into the respective flanking regions of the circular polyribonucleotide.

In some embodiments, a circular polyribonucleotide lacks a 5′-UTR and is competent for protein expression from its one or more expression sequences. In some embodiments, the circular polyribonucleotide lacks a 3′-UTR and is competent for protein expression from its one or more expression sequences. In some embodiments, the circular polyribonucleotide lacks a poly-A sequence and is competent for protein expression from its one or more expression sequences. In some embodiments, the circular polyribonucleotide lacks a termination element and is competent for protein expression from its one or more expression sequences. In some embodiments, the circular polyribonucleotide lacks an internal ribosomal entry site and is competent for protein expression from its one or more expression sequences. In some embodiments, the circular polyribonucleotide lacks a cap and is competent for protein expression from its one or more expression sequences. In some embodiments, the circular polyribonucleotide lacks a 5′-UTR, a 3′-UTR, and an IRES, and is competent for protein expression from its one or more expression sequences. In some embodiments, the circular polyribonucleotide includes one or more of the following sequences: a sequence that encodes one or more miRNAs, a sequence that encodes one or more replication proteins, a sequence that encodes an exogenous gene, a sequence that encodes a therapeutic, a regulatory element (e.g., translation modulator, e.g., translation enhancer or suppressor), a translation initiation sequence, one or more regulatory nucleic acids that targets endogenous genes (e.g., siRNA, lncRNAs, shRNA), and a sequence that encodes a therapeutic mRNA or protein.

In some embodiments, a circular polyribonucleotide lacks a 5′-UTR. In some embodiments, the circular polyribonucleotide lacks a 3′-UTR. In some embodiments, the circular polyribonucleotide lacks a poly-A sequence. In some embodiments, the circular polyribonucleotide lacks a termination element. In some embodiments, the circular polyribonucleotide lacks an internal ribosomal entry site. In some embodiments, the circular polyribonucleotide lacks degradation susceptibility by exonucleases. In some embodiments, the fact that the circular polyribonucleotide lacks degradation susceptibility can mean that the circular polyribonucleotide is not degraded by an exonuclease, or only degraded in the presence of an exonuclease to a limited extent, e.g., that is comparable to or similar to in the absence of exonuclease. In some embodiments, the circular polyribonucleotide is not degraded by exonucleases. In some embodiments, the circular polyribonucleotide has reduced degradation when exposed to exonuclease. In some embodiments, the circular polyribonucleotide lacks binding to a cap-binding protein. In some embodiments, the circular polyribonucleotide lacks a 5′ cap.

### Stagger Elements

In some embodiments, the circular polyribonucleotide includes at least one stagger element adjacent to an expression sequence. In some embodiments, the circular polyribonucleotide includes a stagger element adjacent to each expression sequence. In some embodiments, the stagger element is present on one or both sides of each expression sequence, leading to separation of the expression products, e.g., peptide(s) and or polypeptide(s). In some embodiments, the stagger element is a portion of the one or more expression sequences. In some embodiments, the circular polyribonucleotide includes one or more expression sequences, and each of the one or more expression sequences is separated from a succeeding expression sequence by a stagger element on the circular polyribonucleotide. In some embodiments, the stagger element prevents generation of a single polypeptide (a) from two rounds of translation of a single expression sequence or (b) from one or more rounds of translation of two or more expression sequences. In some embodiments, the stagger element is a sequence separate from the one or more expression sequences. In some embodiments, the stagger element includes a portion of an expression sequence of the one or more expression sequences.

Examples of stagger elements are described in paragraphs [0172]-[0175] of International Patent Publication No. WO2019/118919, which is hereby incorporated by reference in its entirety.

### Non-Coding Sequences

In some embodiments, the polyribonucleotide described herein (e.g., the polyribonucleotide cargo of the polyribonucleotide) includes one or more non-coding sequence, e.g., a sequence that does not encode the expression of polypeptide. In some embodiments, the polyribonucleotide includes two, three, four, five, six, seven, eight, nine, ten or more than ten non-coding sequences. In some embodiments, the polyribonucleotide does not encode a polypeptide expression sequence.

Noncoding sequences can be natural or synthetic sequences. In some embodiments, a noncoding sequence can alter cellular behavior, such as e.g., lymphocyte behavior. In some embodiments, the noncoding sequences are antisense to cellular RNA sequences.

In some embodiments, the polyribonucleotide includes regulatory nucleic acids that are RNA or RNA-like structures typically from about 5-500 base pairs (bp) (depending on the specific RNA structure, e.g., miRNA 5-30 bp, lncRNA 200-500 bp) and may have a nucleobase sequence identical (complementary) or nearly identical (substantially complementary) to a coding sequence in an expressed target gene within the cell. In embodiments, the circular polyribonucleotide includes regulatory nucleic acids that encode an RNA precursor that can be processed to a smaller RNA, e.g., a miRNA precursor, which can be from about 50 to about 1000 bp, that can be processed to a smaller miRNA intermediate or a mature miRNA.

Long non-coding RNAs (lncRNA) are defined as non-protein coding transcripts longer than 100 nucleotides. Many lncRNAs are characterized as tissue specific. Divergent lncRNAs that are transcribed in the opposite direction to nearby protein-coding genes include a significant proportion (e.g., about 20% of total lncRNAs in mammalian genomes) and possibly regulate the transcription of the nearby gene. In one embodiment, the polyribonucleotide provided herein includes a sense strand of a lncRNA. In one embodiment, the polyribonucleotide provided herein includes an antisense strand of a lncRNA.

### Protein-Binding Sequences

In some embodiments, a circular polyribonucleotide includes one or more protein binding sites that enable a protein, e.g., a ribosome, to bind to an internal site in the RNA sequence. By engineering protein binding sites, e.g., ribosome binding sites, into the circular polyribonucleotide, the circular polyribonucleotide may evade or have reduced detection by the host's immune system, have modulated degradation, or modulated translation, by masking the circular polyribonucleotide from components of the host's immune system.

In some embodiments, a circular polyribonucleotide includes at least one immunoprotein binding site, for example to evade immune responses, e.g., CTL (cytotoxic T lymphocyte) responses. In some embodiments, the immunoprotein binding site is a nucleotide sequence that binds to an immunoprotein and aids in masking the circular polyribonucleotide as exogenous. In some embodiments, the immunoprotein binding site is a nucleotide sequence that binds to an immunoprotein and aids in hiding the circular polyribonucleotide as exogenous or foreign.

Traditional mechanisms of ribosome engagement to linear RNA involve ribosome binding to the capped 5′ end of an RNA. From the 5′ end, the ribosome migrates to an initiation codon, whereupon the first peptide bond is formed. According to the present disclosure, internal initiation (i.e., cap-independent) of translation of the circular polyribonucleotide does not require a free end or a capped end. Rather, a ribosome binds to a non-capped internal site, whereby the ribosome begins polypeptide elongation at an initiation codon. In some embodiments, the circular polyribonucleotide includes one or more RNA sequences including a ribosome binding site, e.g., an initiation codon.

Natural 5′UTRs bear features which play roles in for translation initiation. They harbor signatures like Kozak sequences which are commonly known to be involved in the process by which the ribosome initiates translation of many genes. Kozak sequences have the consensus CCR(A/G)CCAUGG (SEQ ID NO: 1), where R is a purine (adenine or guanine) three bases upstream of the start codon (AUG), which is followed by another ‘G’. 5 ′UTR also have been known to form secondary structures which are involved in elongation factor binding.

In some embodiments, a circular polyribonucleotide encodes a protein binding sequence that binds to a protein. In some embodiments, the protein binding sequence targets or localizes the circular polyribonucleotide to a specific target. In some embodiments, the protein binding sequence specifically binds an arginine-rich region of a protein.

In some embodiments, the protein binding site includes, but is not limited to, a binding site to the protein such as ACIN1, AGO, APOBEC3F, APOBEC3G, ATXN2, AUH, BCCIP, CAPRIN1, CELF2, CPSF1, CPSF2, CPSF6, CPSF7, CSTF2, CSTF2T, CTCF, DDX21, DDX3, DDX3X, DDX42, DGCR8, EIF3A, EIF4A3, EIF4G2, ELAVL1, ELAVL3, FAM120A, FBL, FIP1 L1, FKBP4, FMR1, FUS, FXR1, FXR2, GNL3, GTF2F1, HNRNPA1, HNRNPA2B1, HNRNPC, HNRNPK, HNRNPL, HNRNPM, HNRNPU, HNRNPUL1, IGF2BP1, IGF2BP2, IGF2BP3, ILF3, KHDRBS1, LARP7, LIN28A, LIN28B, m6A, MBNL2, METTL3, MOV10, MSI1, MSI2, NONO, NONO-, NOP58, NPM1, NUDT21, PCBP2, POLR2A, PRPF8, PTBP1, RBFOX2, RBM10, RBM22, RBM27, RBM47, RNPS1, SAFB2, SBDS, SF3A3, SF3B4, SIRT7, SLBP, SLTM, SMNDC1, SND1, SRRM4, SRSF1, SRSF3, SRSF7, SRSF9, TAF15, TARDBP, TIA1, TNRC6A, TOP3B, TRA2A, TRA2B, U2AF1, U2AF2, UNK, UPF1, WDR33, XRN2, YBX1, YTHDC1, YTHDF1, YTHDF2, YWHAG, ZC3H7B, PDK1, AKT1, and any other protein that binds RNA.

### Spacer Sequences

In some embodiments, a polyribonucleotide described herein includes one or more spacer sequences. A spacer refers to any contiguous nucleotide sequence (e.g., of one or more nucleotides) that provides distance or flexibility between two adjacent polynucleotide regions. Spacers may be present in between any of the nucleic acid elements described herein. Spacer may also be present within a nucleic acid element described herein.

The spacer may be, e.g., at least 5 (e.g., at least 10, at least 15, at least 20) ribonucleotides in length. In some embodiments, each spacer region is at least 5 (e.g., at least 10, at least 15, at least 20) ribonucleotides in length. Each spacer region may be, e.g., from 5 to 500 (e.g., 10, 20, 30, 40, 50, 60, 70, 80, 90, 100, 150, 200, 250, 300, 350, 400, 450, or 500) ribonucleotides in length. The first spacer region, the second spacer region, or the first spacer region and the second spacer region may include a polyA sequence. The first spacer region, the second spacer region, or the first spacer region and the second spacer region may include a polyA-C sequence. In some embodiments, the first spacer region, the second spacer region, or the first spacer region and the second spacer region includes a polyA-G sequence. In some embodiments, the first spacer region, the second spacer region, or the first spacer region and the second spacer region includes a polyA-T sequence. In some embodiments, the first spacer region, the second spacer region, or the first spacer region and the second spacer region includes a random sequence.

Spacers may also be present within a nucleic acid region described herein. For example, a polynucleotide cargo region may include one or multiple spacers. Spacers may separate regions within the polynucleotide cargo.

In some embodiments, the spacer sequence can be, for example, at least 10 nucleotides in length, at least 15 nucleotides in length, or at least 30 nucleotides in length. In some embodiments, the spacer sequence is at least 7, 8, 9, 10, 11, 12, 13, 14, 15, 16, 17, 18, 19, 20, 25 or 30 nucleotides in length. In some embodiments, the spacer sequence is no more than 100, 90, 80, 70, 60, 50, 45, 40, 35 or 30 nucleotides in length. In some embodiments the spacer sequence is from 20 to 50 nucleotides in length. In certain embodiments, the spacer sequence is 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 nucleotides in length.

The spacer sequences can be polyA sequences, polyA-C sequences, polyC sequences, or poly-U sequences.

In some embodiments, the spacer sequences can be polyA-T, polyA-C, polyA-G, or a random sequence.

A spacer sequences may be used to separate an IRES from adjacent structural elements to martini the structure and function of the IRES or the adjacent element. A spacer can be specifically engineered depending on the IRES. In some embodiments, an RNA folding computer software, such as RNAFold, can be utilized to guide designs of the various elements of the vector, including the spacers.

In some embodiments, the polyribonucleotide includes a 5′ spacer sequence (e.g., between the 5′ annealing region and the polyribonucleotide cargo). In some embodiments, the 5′ spacer sequence is at least 10 nucleotides in length. In another embodiment, the 5′ spacer sequence is at least 15 nucleotides in length. In a further embodiment, the 5′ spacer sequence is at least 30 nucleotides in length. In some embodiments, the 5′ spacer sequence is at least 7, 8, 9, 10, 11, 12, 13, 14, 15, 16, 17, 18, 19, 20, 25 or 30 nucleotides in length. In some embodiments, the 5′ spacer sequence is no more than 100, 90, 80, 70, 60, 50, 45, 40, 35 or 30 nucleotides in length. In some embodiments the 5′ spacer sequence is between 20 and 50 nucleotides in length. In certain embodiments, the 5′ spacer sequence is 10, 11, 12, 13, 14, 15, 16, 17, 18, 19, 20, 21, 22, 23, 24, 25, 26, 27, 28, 29, 30, 31, 32, 33, 34, 35, 3637, 38, 39, 40, 41, 42, 43, 44, 45, 46, 47, 48, 49 or 50 nucleotides in length. In one embodiment, the 5′ spacer sequence is a polyA sequence. In another embodiment, the 5′ spacer sequence is a polyA-C sequence. In some embodiments, the 5′ spacer sequence includes a polyA-G sequence. In some embodiments, the 5′ spacer sequence includes a polyA-T sequence. In some embodiments, the 5′ spacer sequence includes a random sequence.

In some embodiments, the polyribonucleotide includes a 3′ spacer sequence (e.g., between the 3′ annealing region and the polyribonucleotide cargo). In some embodiments, the 3′ spacer sequence is at least 10 nucleotides in length. In another embodiment, the 3′ spacer sequence is at least 15 nucleotides in length. In a further embodiment, the 3′ spacer sequence is at least 30 nucleotides in length. In some embodiments, the 3′ spacer sequence is at least 7, 8, 9, 10, 11, 12, 13, 14, 15, 16, 17, 18, 19, 20, 25 or 30 nucleotides in length. In some embodiments, the 3′ spacer sequence is no more than 100, 90, 80, 70, 60, 50, 45, 40, 35 or 30 nucleotides in length. In some embodiments the 3′ spacer sequence is from 20 to 50 nucleotides in length. In certain embodiments, the 3′ spacer sequence is 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 nucleotides in length. In one embodiment, the 3′ spacer sequence is a polyA sequence. In another embodiment, the 5′ spacer sequence is a polyA-C sequence. In some embodiments, the 5′ spacer sequence includes a polyA-G sequence. In some embodiments, the 5′ spacer sequence includes a polyA-T sequence. In some embodiments, the 5′ spacer sequence includes a random sequence.

In one embodiment, the polyribonucleotide includes a 5′ spacer sequence, but not a 3′ spacer sequence. In another embodiment, the polyribonucleotide includes a 3′ spacer sequence, but not a 5′ spacer sequence. In another embodiment, the polyribonucleotide includes neither a 5′ spacer sequence, nor a 3′ spacer sequence. In another embodiment, the polyribonucleotide does not include an IRES sequence. In a further embodiment, the polyribonucleotide does not include an IRES sequence, a 5′ spacer sequence or a 3′ spacer sequence.

In some embodiments, the spacer sequence includes at least 3 ribonucleotides, at least 4 ribonucleotides, at least 5 ribonucleotides, at least about 8 ribonucleotides, at least about 10 ribonucleotides, at least about 12 ribonucleotides, at least about 15 ribonucleotides, at least about 20 ribonucleotides, at least about 25 ribonucleotides, at least about 30 ribonucleotides, at least about 40 ribonucleotides, at least about 50 ribonucleotides, at least about 60 ribonucleotides, at least about 70 ribonucleotides, at least about 80 ribonucleotides, at least about 90 ribonucleotides, at least about 100 ribonucleotides, at least about 120 ribonucleotides, at least about 150 ribonucleotides, at least about 200 ribonucleotides, at least about 250 ribonucleotides, at least about 300 ribonucleotides, at least about 400 ribonucleotides, at least about 500 ribonucleotides, at least about 600 ribonucleotides, at least about 700 ribonucleotides, at least about 800 ribonucleotides, at least about 900 ribonucleotides, or at least about 100 ribonucleotides.

### Bioreactors

In some embodiments, any method of purifying a polyribonucleotide (e.g., circular polyribonucleotide) described herein may be performed in a bioreactor. A bioreactor refers to any vessel in which a chemical or biological process is carried out which involves organisms or biochemically active substances derived from such organisms. Bioreactors may be compatible with the cell-free methods for purifying or producing circular RNA described herein. A vessel for a bioreactor may include a culture flask, a dish, or a bag that may be single use (disposable), autoclavable, or sterilizable. A bioreactor may be made of glass, or it may be polymer-based, or it may be made of other materials.

Examples of bioreactors include, without limitation, stirred tank (e.g., well mixed) bioreactors and tubular (e.g., plug flow) bioreactors, airlift bioreactors, membrane stirred tanks, spin filter stirred tanks, vibromixers, fluidized bed reactors, and membrane bioreactors. The mode of operating the bioreactor may be a batch or continuous processes. A bioreactor is continuous when the reagent and product streams are continuously being fed and withdrawn from the system. A batch bioreactor may have a continuous recirculating flow, but no continuous feeding of reagents or product harvest.

Some methods of the present disclosure are directed to large-scale production of polyribonucleotides. For large-scale production methods, the method may be performed in a volume of 1 liter (L) to 50 L, or more (e.g., 5 L, 10 L, 15 L, 20 L, 25 L, 30 L, 35 L, 40 L, 45 L, 50 L, or more). In some embodiments, the method may be performed in a volume of 5 L to 10 L, 5 L to 15 L, 5 L to 20 L, 5 L to 25 L, 5 L to 30 L, 5 L to 35 L, 5 L to 40 L, 5 L to 45 L, 10 L to 15 L, 10 L to 20 L, 10 L to 25 L, 20 L to 30 L, 10 L to 35 L, 10 L to 40 L, 10L to 45 L, 10L to 50 L, 15L to 20 L, 15L to 25 L, 15 L to 30 L, 15 L to 35 L, 15 L to 40 L, 15 L to 45 L, or 15 to 50 L.

In some embodiments, a bioreactor may produce at least 1 g of RNA. In some embodiments, a bioreactor may produce 1-200 g of RNA (e.g., 1-10 g, 1-20 g, 1-50 g, 10-50 g, 10-100 g, 50-100 g, of 50-200 g of RNA). In some embodiments, the amount produced is measured per liter (e.g., 1-200 g per liter), per batch or reaction (e.g., 1-200 g per batch or reaction), or per unit time (e.g., 1-200 g per hour or per day).

In some embodiments, more than one bioreactor may be utilized in series to increase the production capacity (e.g., one, two, three, four, five, six, seven, eight, or nine bioreactors may be used in series).

### Methods of Use

In some embodiments, the polyribonucleotides (e.g., circular polyribonucleotides) made as described herein are used as effectors in therapy or agriculture.

For example, a polyribonucleotide purified by the methods described herein may be administered to a subject (e.g., in a pharmaceutical, veterinary, or agricultural composition). In some embodiments, the subject is a vertebrate animal (e.g., mammal, bird, fish, reptile, or amphibian). In some embodiments, the subject is a human. In some embodiments, the subject is a non-human mammal. In embodiments, the subject is a non-human mammal is such as a non-human primate (e.g., monkeys, apes), ungulate (e.g., cattle, buffalo, sheep, goat, pig, camel, llama, alpaca, deer, horses, donkeys), carnivore (e.g., dog, cat), rodent (e.g., rat, mouse), or lagomorph (e.g., rabbit). In embodiments, the subject is a bird, such as a member of the avian taxa Galliformes (e.g., chickens, turkeys, pheasants, quail), Anseriformes (e.g., ducks, geese), Paleaognathae (e.g., ostriches, emus), Columbiformes (e.g., pigeons, doves), or Psittaciformes (e.g., parrots). In embodiments, the subject is an invertebrate such as an arthropod (e.g., insects, arachnids, crustaceans), a nematode, an annelid, a helminth, or a mollusk. In embodiments, the subject is an invertebrate agricultural pest or an invertebrate that is parasitic on an invertebrate or vertebrate host. In embodiments, the subject is a plant, such as an angiosperm plant (which can be a dicot or a monocot) or a gymnosperm plant (e.g., a conifer, a cycad, a gnetophyte, a Ginkgo), a fern, horsetail, clubmoss, or a bryophyte. In embodiments, the subject is a eukaryotic alga (unicellular or multicellular). In embodiments, the subject is a plant of agricultural or horticultural importance, such as row crop plants, fruit-producing plants and trees, vegetables, trees, and ornamental plants including ornamental flowers, shrubs, trees, groundcovers, and turf grasses.

In some embodiments, the disclosure provides a method of modifying a subject by providing to the subject a composition or formulation described herein. In some embodiments, the composition or formulation is or includes a nucleic acid molecule (e.g., a DNA molecule or an RNA molecule described herein), and the polynucleotide is provided to a eukaryotic subject. In some embodiments, the composition or formulation is or includes or a eukaryotic or prokaryotic cell including a nucleic acid described herein.

In some embodiments, the disclosure provides a method of treating a condition in a subject in need thereof by providing to the subject a composition or formulation described herein. In some embodiments, the composition or formulation is or includes a nucleic acid molecule (e.g., a DNA molecule or a polyribonucleotide described herein), and the polynucleotide is provided to a eukaryotic subject. In some embodiments, the composition or formulation is or includes a eukaryotic or prokaryotic cell including a nucleic acid described herein.

In some embodiments, the disclosure provides a method of providing a polyribonucleotide (e.g., circular polyribonucleotide) to a subject by providing a eukaryotic or prokaryotic cell include a polynucleotide described herein to the subject.

### Formulations

In some embodiments of the present disclosure a polyribonucleotide (e.g., a circular polyribonucleotide) described herein may be formulated in composition, e.g., a composition for delivery to a cell, a plant, an invertebrate animal, a non-human vertebrate animal, or a human subject, e.g., an agricultural, veterinary, or pharmaceutical composition. In some embodiments, the polyribonucleotide is formulated in a pharmaceutical composition. In some embodiments, a composition includes a polyribonucleotide and a diluent, a carrier, an adjuvant, or a combination thereof. In a particular embodiment, a composition includes a polyribonucleotide described herein and a carrier or a diluent free of any carrier. In some embodiments, a composition including a polyribonucleotide with a diluent free of any carrier is used for naked delivery of the polyribonucleotide (e.g., circular polyribonucleotide) to a subject.

Pharmaceutical compositions may optionally include one or more additional active substances, e.g., therapeutically and/or prophylactically active substances. Pharmaceutical compositions may optionally include an inactive substance that serves as a vehicle or medium for the compositions described herein (e.g., compositions including circular polyribonucleotides, such as any one of the inactive ingredients approved by the United States Food and Drug Administration (FDA) and listed in the Inactive Ingredient Database). Pharmaceutical compositions of the present invention may be sterile and/or pyrogen-free. General considerations in the formulation and/or manufacture of pharmaceutical agents may be found, for example, in Remington: The Science and Practice of Pharmacy 21st ed., Lippincott Williams & Wilkins, 2005 (incorporated herein by reference). Non-limiting examples of an inactive substance include solvents, aqueous solvents, non-aqueous solvents, dispersion media, diluents, dispersions, suspension aids, surface active agents, isotonic agents, thickening agents, emulsifying agents, preservatives, polymers, peptides, proteins, cells, hyaluronidases, dispersing agents, granulating agents, disintegrating agents, binding agents, buffering agents (e.g., phosphate buffered saline (PBS)), lubricating agents, oils, and mixtures thereof.

Although the descriptions of pharmaceutical compositions provided herein are principally directed to pharmaceutical compositions which are suitable for administration to humans, it will be understood by the skilled artisan that such compositions are suitable for administration to any other animal, e.g., to non-human animals, e.g., non-human mammals. Modification of pharmaceutical compositions suitable for administration to humans to render the compositions suitable for administration to various animals is well understood, and the ordinarily skilled veterinary pharmacologist can design and/or perform such modification with merely ordinary, if any, experimentation. Subjects to which administration of the pharmaceutical compositions is contemplated include, but are not limited to, humans and/or other primates; mammals, including commercially relevant mammals such as cattle, pigs, horses, sheep, cats, dogs, mice, and/or rats; and/or birds, including commercially relevant birds such as poultry, chickens, ducks, geese, and/or turkeys.

Formulations of the pharmaceutical compositions described herein may be prepared by any method known or hereafter developed in the art of pharmacology. In general, such preparatory methods include the step of bringing the active ingredient into association with an excipient and/or one or more other accessory ingredients, and then, if necessary and/or desirable, dividing, shaping and/or packaging the product.

In some embodiments, the reference criterion for the amount of linear polyribonucleotide molecules present in the preparation is the presence of no more than 1 ng/ml, 5 ng/ml, 10 ng/ml, 15 ng/ml, 20 ng/ml, 25 ng/ml, 30 ng/ml, 35 ng/ml, 40 ng/ml, 50 ng/ml, 60 ng/ml, 70 ng/ml, 80 ng/ml, 90 ng/ml, 100 ng/ml, 200 ng/ml, 300 ng/ml, 400 ng/ml, 500 ng/ml, 600 ng/ml, 1 μg/ml, 10 μg/ml, 50 μg/ml, 100 μg/ml, 200 g/ml, 300 μg/ml, 400 μg/ml, 500 μg/ml, 600 μg/ml, 700 μg/ml, 800 μg/ml, 900 μg/ml, 1 mg/ml, 1.5 mg/ml, or 2 mg/ml of linear polyribonucleotide molecules.

In some embodiments, the reference criterion for the amount of circular polyribonucleotide molecules present in the preparation is at least 30% (w/w), 40% (w/w), 50% (w/w), 60% (w/w), 70% (w/w), 80% (w/w), 85% (w/w), 90% (w/w), 91% (w/w), 92% (w/w), 93% (w/w), 94% (w/w), 95% (w/w), 96% (w/w), 97% (w/w), 98% (w/w), 99% (w/w), 99.1% (w/w), 99.2% (w/w), 99.3% (w/w), 99.4% (w/w), 99.5% (w/w), 99.6% (w/w), 99.7% (w/w), 99.8% (w/w), 99.9% (w/w), or 100% (w/w) molecules of the total ribonucleotide molecules in the pharmaceutical preparation.

In some embodiments, the reference criterion for the amount of linear polyribonucleotide molecules present in the preparation is no more than 0.5% (w/w), 1% (w/w), 2% (w/w), 5% (w/w), 10% (w/w), 15% (w/w), 20% (w/w), 25% (w/w), 30% (w/w), 40% (w/w), 50% (w/w) linear polyribonucleotide molecules of the total ribonucleotide molecules in the pharmaceutical preparation.

In some embodiments, the reference criterion for the amount of nicked polyribonucleotide molecules present in the preparation is no more than 0.5% (w/w), 1% (w/w), 2% (w/w), 5% (w/w), 10% (w/w), or 15% (w/w) nicked polyribonucleotide molecules of the total ribonucleotide molecules in the pharmaceutical preparation.

In some embodiments, the reference criterion for the amount of combined nicked and linear polyribonucleotide molecules present in the preparation is no more than 0.5% (w/w), 1% (w/w), 2% (w/w), 5% (w/w), 10% (w/w), 15% (w/w), 20% (w/w), 25% (w/w), 30% (w/w), 40% (w/w), 50% (w/w) combined nicked and linear polyribonucleotide molecules of the total ribonucleotide molecules in the pharmaceutical preparation. In some embodiments, a pharmaceutical preparation is an intermediate pharmaceutical preparation of a final circular polyribonucleotide drug product. In some embodiments, a pharmaceutical preparation is a drug substance or active pharmaceutical ingredient (API). In some embodiments, a pharmaceutical preparation is a drug product for administration to a subject.

In some embodiments, a preparation of circular polyribonucleotides is (before, during or after the reduction of linear RNA) further processed to substantially remove DNA, protein contamination, impurities, or by-products (e.g., cell protein such as a host cell protein or protein process impurities), endotoxin, mononucleotide molecules, and/or a process-related impurity.

### Preservatives

A composition or pharmaceutical composition provided herein can include material for a single administration, or can include material for multiple administrations (e.g., a “multidose” kit). The polyribonucleotide can be present in either linear or circular form. The composition or pharmaceutical composition can include one or more preservatives such as thiomersal or 2-phenoxyethanol. Preservatives can be used to prevent microbial contamination during use. Suitable preservatives include: benzalkonium chloride, thimerosal, chlorobutanol, methyl paraben, propyl paraben, phenylethyl alcohol, edetate disodium, sorbic acid, Onamer M, or other agents known to those skilled in the art. In ophthalmic products, e.g., such preservatives can be employed at a level of from 0.004% to 0.02%. In the compositions described herein the preservative, e.g., benzalkonium chloride, can be employed at a level of from 0.001% to less than 0.01%, e.g., from 0.001% to 0.008%, preferably about 0.005% by weight.

Polyribonucleotides can be susceptible to RNase that can be abundant in ambient environment. Compositions provided herein can include reagents that inhibit RNase activity, thereby preserving the polyribonucleotide from degradation. In some cases, the composition or pharmaceutical composition includes any RNase inhibitor known to one skilled in the art. Alternatively or additionally, the polyribonucleotide, and cell-penetrating agent and/or pharmaceutically acceptable diluents or carriers, vehicles, excipients, or other reagents in the composition provided herein can be prepared in RNase-free environment. The composition can be formulated in RNase-free environment.

In some cases, a composition provided herein can be sterile. The composition can be formulated as a sterile solution or suspension, in suitable vehicles, known in the art. The composition can be sterilized by conventional, known sterilization techniques, e.g., the composition can be sterile filtered.

### Salts

In some cases, a composition or pharmaceutical composition provided herein includes one or more salts. For controlling the tonicity, a physiological salt such as sodium salt can be included a composition provided herein. Other salts can include potassium chloride, potassium dihydrogen phosphate, disodium phosphate, and/or magnesium chloride, or the like. In some cases, the composition is formulated with one or more pharmaceutically acceptable salts. The one or more pharmaceutically acceptable salts can include those of the inorganic ions, such as, for example, sodium, potassium, calcium, magnesium ions, and the like. Such salts can include salts with inorganic or organic acids, such as hydrochloric acid, hydrobromic acid, phosphoric acid, nitric acid, sulfuric acid, methane sulfonic acid, p-toluenesulfonic acid, acetic acid, fumaric acid, succinic acid, lactic acid, mandelic acid, malic acid, citric acid, tartaric acid, or maleic acid. The polyribonucleotide can be present in either linear or circular form.

### Buffers/pH

A composition or pharmaceutical composition provided herein can include one or more buffers, such as a Tris buffer; a borate buffer; a succinate buffer; a histidine buffer (e.g., with an aluminum hydroxide adjuvant); or a citrate buffer. Buffers, in some cases, are included in the 5-20 mM range.

A composition or pharmaceutical composition provided herein can have a pH between about 5.0 and about 8.5, between about 6.0 and about 8.0, between about 6.5 and about 7.5, or between about 7.0 and about 7.8. The composition or pharmaceutical composition can have a pH of about 7. The polyribonucleotide can be present in either linear or circular form.

### Detergents/surfactants

A composition or pharmaceutical composition provided herein can include one or more detergents and/or surfactants, depending on the intended administration route, e.g., polyoxyethylene sorbitan esters surfactants (commonly referred to as “Tweens”), e.g., polysorbate 20 and polysorbate 80; copolymers of ethylene oxide (EO), propylene oxide (PO), and/or butylene oxide (BO), sold under the DOWFAX™ tradename, such as linear EO/PO block copolymers; octoxynols, which can vary in the number of repeating ethoxy (oxy-1,2-ethanediyl) groups, e.g., octoxynol-9 (Triton X-100, or t-octylphenoxypolyethoxyethanol); (octyl phenoxy)polyethoxyethanol (IGEPAL CA-630/NP-40); phospholipids such as phosphatidylcholine (lecithin); nonylphenol ethoxylates, such as the Tergitol™ NP series; polyoxyethylene fatty ethers derived from lauryl, cetyl, stearyl and oleyl alcohols (known as Brij surfactants), such as triethyleneglycol monolauryl ether (Brij 30); and sorbitan esters (commonly known as “SPANs”), such as sorbitan trioleate (Span 85) and sorbitan monolaurate, an octoxynol (such as octoxynol-9 (Triton X-100) or t-octylphenoxypolyethoxyethanol), a cetyl trimethyl ammonium bromide (“CTAB”), or sodium deoxycholate. The one or more detergents and/or surfactants can be present only at trace amounts. In some cases, the composition can include less than 1 mg/ml of each of octoxynol-10 and polysorbate 80. Non-ionic surfactants can be used herein. Surfactants can be classified by their “HLB” (hydrophile/lipophile balance). In some cases, surfactants have a HLB of at least 10, at least 15, and/or at least 16. The polyribonucleotide can be present in either linear or circular form.

### Diluents

In some embodiments, a composition of the disclosure includes a polyribonucleotide and a diluent. In some embodiments, a composition of the disclosure includes a linear polyribonucleotide and a diluent.

A diluent can be a non-carrier excipient. A non-carrier excipient serves as a vehicle or medium for a composition, such as a circular polyribonucleotide as described herein. A non-carrier excipient serves as a vehicle or medium for a composition, such as a linear polyribonucleotide as described herein. Non-limiting examples of a non-carrier excipient include solvents, aqueous solvents, non-aqueous solvents, dispersion media, diluents, dispersions, suspension aids, surface active agents, isotonic agents, thickening agents, emulsifying agents, preservatives, polymers, peptides, proteins, cells, hyaluronidases, dispersing agents, granulating agents, disintegrating agents, binding agents, buffering agents (e.g., phosphate buffered saline (PBS)), lubricating agents, oils, and mixtures thereof. A non-carrier excipient can be any one of the inactive ingredients approved by the United States Food and Drug Administration (FDA) and listed in the Inactive Ingredient Database that does not exhibit a cell-penetrating effect. A non-carrier excipient can be any inactive ingredient suitable for administration to a non-human animal, for example, suitable for veterinary use. Modification of compositions suitable for administration to humans in order to render the compositions suitable for administration to various animals is well understood, and the ordinarily skilled veterinary pharmacologist can design and/or perform such modification with merely ordinary, if any, experimentation.

In some embodiments, the polyribonucleotide (e.g., circular polyribonucleotide) may be delivered as a naked delivery formulation, such as including a diluent. A naked delivery formulation delivers a polyribonucleotide, to a cell without the aid of a carrier and without modification or partial or complete encapsulation of the polyribonucleotide, capped polyribonucleotide, or complex thereof.

A naked delivery formulation is a formulation that is free from a carrier and wherein the polyribonucleotide (e.g., circular polyribonucleotide) is without a covalent modification that binds a moiety that aids in delivery to a cell or without partial or complete encapsulation of the polyribonucleotide. In some embodiments, a polyribonucleotide without a covalent modification that binds a moiety that aids in delivery to a cell is a polyribonucleotide that is not covalently bound to a protein, small molecule, a particle, a polymer, or a biopolymer. A polyribonucleotide without covalent modification that binds a moiety that aids in delivery to a cell does not contain a modified phosphate group. For example, a polyribonucleotide without a covalent modification that binds a moiety that aids in delivery to a cell does not contain phosphorothioate, phosphoroselenates, boranophosphates, boranophosphate esters, hydrogen phosphonates, phosphoramidates, phosphorodiamidates, alkyl or aryl phosphonates, or phosphotriesters.

In some embodiments, a naked delivery formulation is free of any or all of transfection reagents, cationic carriers, carbohydrate carriers, nanoparticle carriers, or protein carriers. In some embodiments, a naked delivery formulation is free from phtoglycogen octenyl succinate, phytoglycogen beta-dextrin, anhydride-modified phytoglycogen beta-dextrin, lipofectamine, polyethylenimine, poly(trimethylenimine), poly(tetra methylenimine), polypropylenimine, aminoglycoside-polyamine, dideoxy-diamino-b-cyclodextrin, spermine, spermidine, poly(2-dimethylamino)ethyl methacrylate, poly(lysine), poly(histidine), poly(arginine), cationized gelatin, dendrimers, chitosan, 1,2-Dioleoyl-3-Trimethylammonium-Propane(DOTAP), N-[1-(2,3-dioleoyloxy)propyl]-N,N,N- trimethylammonium chloride (DOTMA), 1-[2-(oleoyloxy)ethyl]-2-oleyl-3-(2-hydroxyethyl)imidazolinium chloride (DOTIM), 2,3-dioleyloxy-N-[2(sperminecarboxamido)ethyl]-N,N-dimethyl-1-propanaminium trifluoroacetate (DOSPA), 3B-[N-(N\N′-Dimethylaminoethane)-carbamoyl]Cholesterol Hydrochloride (DC-Cholesterol HCl), diheptadecylamidoglycyl spermidine (DOGS), N,N-distearyl-N,N-dimethylammonium bromide (DDAB), N-(1,2-dimyristyloxyprop-3-yl)-N,N-dimethyl-N-hydroxyethyl ammonium bromide (DMRIE), N,N-dioleyl-N,N-dimethylammonium chloride (DODAC), human serum albumin (HSA), low-density lipoprotein (LDL), high-density lipoprotein (HDL), or globulin.

In certain embodiments, a naked delivery formulation includes a non-carrier excipient. In some embodiments, a non-carrier excipient includes an inactive ingredient that does not exhibit a cell-penetrating effect. In some embodiments, a non-carrier excipient includes a buffer, for example PBS. In some embodiments, a non-carrier excipient is a solvent, a non-aqueous solvent, a diluent, a suspension aid, a surface-active agent, an isotonic agent, a thickening agent, an emulsifying agent, a preservative, a polymer, a peptide, a protein, a cell, a hyaluronidase, a dispersing agent, a granulating agent, a disintegrating agent, a binding agent, a buffering agent, a lubricating agent, or an oil.

In some embodiments, a naked delivery formulation includes a diluent. A diluent may be a liquid diluent or a solid diluent. In some embodiments, a diluent is an RNA solubilizing agent, a buffer, or an isotonic agent. Examples of an RNA solubilizing agent include water, ethanol, methanol, acetone, formamide, and 2-propanol. Examples of a buffer include 2-(N-morpholino)ethanesulfonic acid (MES), Bis-Tris, 2-[(2-amino-2-oxoethyl)-(carboxymethyl)amino]acetic acid (ADA), N-(2-Acetamido)-2-aminoethanesulfonic acid (ACES), piperazine-N,N′-bis(2-ethanesulfonic acid) (PIPES), 2-[[1,3-dihydroxy-2-(hydroxymethyl)propan-2-yl]amino]ethanesulfonic acid (TES), 3-(N-morpholino)propane sulfonic acid (MOPS), 4-(2-hydroxyethyl)-1-piperazineethanesulfonic acid (HEPES), Tris, Tricine, Gly-Gly, Bicine, or phosphate. Examples of an isotonic agent include glycerin, mannitol, polyethylene glycol, propylene glycol, trehalose, or sucrose.

### Carriers

In some embodiments, a composition of the disclosure includes a circular polyribonucleotide and a carrier. In some embodiments, a composition of the disclosure includes a linear polyribonucleotide and a carrier.

In certain embodiments, a composition includes a circular polyribonucleotide as described herein in a vesicle or other membrane-based carrier. In certain embodiments, a composition includes a linear polyribonucleotide as described herein in a vesicle or other membrane-based carrier.

In other embodiments, a composition includes the circular polyribonucleotide in or via a cell, vesicle or other membrane-based carrier. In other embodiments, a composition includes the linear polyribonucleotide in or via a cell, vesicle or other membrane-based carrier. In one embodiment, a composition includes the circular polyribonucleotide in liposomes or other similar vesicles. In one embodiment, a composition includes the linear polyribonucleotide in liposomes or other similar vesicles. Liposomes are spherical vesicle structures composed of a uni- or multilamellar lipid bilayer surrounding internal aqueous compartments and a relatively impermeable outer lipophilic phospholipid bilayer. Liposomes may be anionic, neutral, or cationic. Liposomes are biocompatible, nontoxic, can deliver both hydrophilic and lipophilic drug molecules, protect their cargo from degradation by plasma enzymes, and transport their load across biological membranes and the blood brain barrier (BBB) (see, e.g., Spuch and Navarro, Journal of Drug Delivery, vol. 2011, Article ID 469679, 12 pages, 2011. doi:10.1155/2011/469679 for review).

Vesicles can be made from several different types of lipids; however, phospholipids are most commonly used to generate liposomes as drug carriers. Methods for preparation of multilamellar vesicle lipids are known in the art (see for example U.S. Pat. No. 6,693,086, the teachings of which relating to multilamellar vesicle lipid preparation are incorporated herein by reference). Although vesicle formation can be spontaneous when a lipid film is mixed with an aqueous solution, it can also be expedited by applying force in the form of shaking by using a homogenizer, sonicator, or an extrusion apparatus (see, e.g., Spuch and Navarro, Journal of Drug Delivery, vol. 2011, Article ID 469679, 12 pages, 2011. doi:10.1155/2011/469679 for review). Extruded lipids can be prepared by extruding through filters of decreasing size, as described in Templeton et al., Nature Biotechnol., 15:647-52, 1997, the teachings of which relating to extruded lipid preparation are incorporated herein by reference.

In certain embodiments, a composition of the disclosure includes a polyribonucleotide and lipid nanoparticles, for example lipid nanoparticles described herein. In certain embodiments, a composition of the disclosure includes a linear polyribonucleotide and lipid nanoparticles. Lipid nanoparticles are another example of a carrier that provides a biocompatible and biodegradable delivery system for a polyribonucleotide molecule as described herein. Lipid nanoparticles are another example of a carrier that provides a biocompatible and biodegradable delivery system for a linear polyribonucleotide molecule as described herein. Nanostructured lipid carriers (NLCs) are modified solid lipid nanoparticles (SLNs) that retain the characteristics of the SLN, improve drug stability and loading capacity, and prevent drug leakage. Polymer nanoparticles (PNPs) are an important component of drug delivery. These nanoparticles can effectively direct drug delivery to specific targets and improve drug stability and controlled drug release. Lipid-polymer nanoparticles (PLNs), a new type of carrier that combines liposomes and polymers, may also be employed. These nanoparticles possess the complementary advantages of PNPs and liposomes. A PLN is composed of a core-shell structure; the polymer core provides a stable structure, and the phospholipid shell offers good biocompatibility. As such, the two components increase the drug encapsulation efficiency rate, facilitate surface modification, and prevent leakage of water-soluble drugs. For a review, see, e.g., Li et al. 2017, Nanomaterials 7, 122; doi:10.3390/nano7060122.

Additional non-limiting examples of carriers include carbohydrate carriers (e.g., an anhydride-modified phytoglycogen or glycogen-type material), protein carriers (e.g., a protein covalently linked to the polyribonucleotide or a protein covalently linked to the linear polyribonucleotide), or cationic carriers (e.g., a cationic lipopolymer or transfection reagent). Non-limiting examples of carbohydrate carriers include phtoglycogen octenyl succinate, phytoglycogen beta-dextrin, and anhydride-modified phytoglycogen beta-dextrin. Non-limiting examples of cationic carriers include lipofectamine, polyethylenimine, poly(trimethylenimine), poly(tetra ethylenimine), polypropylenimine, aminoglycoside-polyamine, dideoxy-diamino-b-cyclodextrin, spermine, spermidine, poly(2-dimethylamino)ethyl methacrylate, poly(lysine), poly(histidine), poly(arginine), cationized gelatin, dendrimers, chitosan, 1,2-Dioleoyl-3-Trimethylammonium-Propane(DOTAP), N-[1-(2,3-dioleoyloxy)propyl]-N,N,N-trimethylammonium chloride (DOTMA), 1-[2-(oleoyloxy)ethyl]-2-oleyl-3-(2-hydroxyethyl)imidazolinium chloride (DOTIM), 2,3-dioleyloxy-N-[2(sperminecarboxamido)ethyl]-N,N-dimethyl-1-propanaminium trifluoroacetate (DOSPA), 3B-[N-(N\N′-Dimethylaminoethane)-carbamoyl]Cholesterol Hydrochloride (DC-Cholesterol HCl), diheptadecylamidoglycyl spermidine (DOGS), N,N-distearyl-N,N-dimethylammonium bromide (DDAB), N-(1,2-dimyristyloxyprop-3-yl)-N,N-dimethyl-N-hydroxyethyl ammonium bromide (DMRIE), and N,N-dioleyl-N,N-dimethylammonium chloride (DODAC). Non-limiting examples of protein carriers include human serum albumin (HSA), low-density lipoprotein (LDL), high-density lipoprotein (HDL), or globulin.

Exosomes can also be used as drug delivery vehicles for a composition or preparation described herein. Exosomes can be used as drug delivery vehicles for a linear polyribonucleotide composition or preparation described herein. For a review, see Ha et al. July 2016. Acta Pharmaceutica Sinica B. Volume 6, Issue 4, Pages 287-96; doi.org/10.1016/j.apsb.2016.02.001.

Ex vivo differentiated red blood cells can also be used as a carrier for a composition or preparation described herein. Ex vivo differentiated red blood cells can also be used as a carrier for a linear polyribonucleotide composition or preparation described herein. See, e.g., International Patent Publication Nos. WO2015/073587; WO2017/123646; WO2017/123644; WO2018/102740; WO2016/183482; WO2015/153102; WO2018/151829; WO2018/009838; Shi et al. 2014. Proc Natl Acad Sci USA. 111(28): 10131-10136; U.S. Pat. No. 9,644,180; Huang et al. 2017. Nature Communications 8: 423; Shi et al. 2014. Proc Natl Acad Sci USA. 111(28): 10131-10136.

Fusosome compositions, e.g., as described in International Patent Publication No. WO2018/208728, can also be used as carriers to deliver a polyribonucleotide molecule described herein. Fusosome compositions, e.g., as described in WO2018/208728, can also be used as carriers to deliver a linear polyribonucleotide molecule described herein.

Virosomes and virus-like particles (VLPs) can also be used as carriers to deliver a polyribonucleotide molecule described herein to targeted cells. Virosomes and virus-like particles (VLPs) can also be used as carriers to deliver a linear polyribonucleotide molecule described herein to targeted cells.

Plant nanovesicles and plant messenger packs (PMPs), e.g., as described in International Patent Publication Nos. WO2011/097480, WO2013/070324, WO2017/004526, or WO2020/041784 can also be used as carriers to deliver the composition or preparation described herein. Plant nanovesicles and plant messenger packs (PMPs) can also be used as carriers to deliver a linear polyribonucleotide composition or preparation described herein.

Microbubbles can also be used as carriers to deliver a polyribonucleotide molecule described herein. Microbubbles can also be used as carriers to deliver a linear polyribonucleotide molecule described herein. See, e.g., U.S. Pat. No. 7,115,583; Beeri, R. et al., Circulation. 2002 Oct. 1; 106(14):1756-1759; Bez, M. et al., Nat Protoc. 2019 April; 14(4): 1015-1026; Hernot, S. et al., Adv Drug Deliv Rev. 2008 Jun. 30; 60(10): 1153-1166; Rychak, J. J. et al., Adv Drug Deliv Rev. 2014 June; 72: 82-93. In some embodiments, microbubbles are albumin-coated perfluorocarbon microbubbles.

The carrier including the polyribonucleotides described herein may include a plurality of particles. The particles may have median article size of 30 to 700 nanometers (e.g., 30 to 50, 50 to 100, 100 to 200, 200 to 300, 300 to 400, 400 to 500, 500 to 600, 600 to 700, 100 to 500, 50 to 500, or 200 to 700 nanometers). The size of the particle may be optimized to favor deposition of the payload, including the polyribonucleotide into a cell. Deposition of the polyribonucleotide into certain cell types may favor different particle sizes. For example, the particle size may be optimized for deposition of the polyribonucleotide into antigen presenting cells. The particle size may be optimized for deposition of the polyribonucleotide into dendritic cells. Additionally, the particle size may be optimized for depositions of the polyribonucleotide into draining lymph node cells.

### Lipid Nanoparticles

The compositions, methods, and delivery systems provided by the present disclosure may employ any suitable carrier or delivery modality described herein, including, in certain embodiments, lipid nanoparticles (LNPs). Lipid nanoparticles, in some embodiments, include one or more ionic lipids, such as non-cationic lipids (e.g., neutral or anionic, or zwitterionic lipids); one or more conjugated lipids (such as PEG-conjugated lipids or lipids conjugated to polymers described in Table 5 of WO2019217941; incorporated herein by reference in its entirety); one or more sterols (e.g., cholesterol).

Lipids that can be used in nanoparticle formations (e.g., lipid nanoparticles) include, for example those described in Table 4 of WO2019217941, which is incorporated by reference—e.g., a lipid-containing nanoparticle can include one or more of the lipids in Table 4 of WO2019217941. Lipid nanoparticles can include additional elements, such as polymers, such as the polymers described in Table 5 of WO2019217941, incorporated by reference.

In some embodiments, conjugated lipids, when present, can include one or more of PEG-diacylglycerol (DAG) (such as 1-(monomethoxy-polyethyleneglycol)-2,3-dimyristoylglycerol (PEG-DMG)), PEG-dialkyloxypropyl (DAA), PEG-phospholipid, PEG-ceramide (Cer), a pegylated phosphatidylethanoloamine (PEG-PE), PEG succinate diacylglycerol (PEGS-DAG) (such as 4-0-(2′,3′-di(tetradecanoyloxy)propyl-1-0-(w-methoxy(polyethoxy)ethyl) butanedioate (PEG-S-DMG)), PEG dialkoxypropylcarbam, N-(carbonyl-methoxypoly ethylene glycol 2000)-1,2-distearoyl-sn-glycero-3-phosphoethanolamine sodium salt, and those described in Table 2 of WO2019051289 (incorporated by reference), and combinations of the foregoing.

In some embodiments, sterols that can be incorporated into lipid nanoparticles include one or more of cholesterol or cholesterol derivatives, such as those in WO2009/127060 or US2010/0130588, which are incorporated by reference. Additional exemplary sterols include phytosterols, including those described in Eygeris et al. (2020), dx.doi.org/10.1021/acs.nanolett.0c01386, incorporated herein by reference.

In some embodiments, the lipid particle includes an ionizable lipid, a non-cationic lipid, a conjugated lipid that inhibits aggregation of particles, and a sterol. The amounts of these components can be varied independently and to achieve desired properties. For example, in some embodiments, the lipid nanoparticle includes an ionizable lipid is in an amount from about 20 mol % to about 90 mol % of the total lipids (in other embodiments it may be 20-70% (mol), 30-60% (mol) or 40-50% (mol); about 50 mol % to about 90 mol % of the total lipid present in the lipid nanoparticle), a non-cationic lipid in an amount from about 5 mol % to about 30 mol % of the total lipids, a conjugated lipid in an amount from about 0.5 mol % to about 20 mol % of the total lipids, and a sterol in an amount from about 20 mol % to about 50 mol % of the total lipids. The ratio of total lipid to nucleic acid can be varied as desired. For example, the total lipid to nucleic acid (mass or weight) ratio can be from about 10:1 to about 30:1.

In some embodiments, the lipid to nucleic acid ratio (mass/mass ratio; w/w ratio) can be in the range of from about 1:1 to about 25:1, from about 10:1 to about 14:1, from about 3:1 to about 15:1, from about 4:1 to about 10:1, from about 5:1 to about 9:1, or about 6:1 to about 9:1. The amounts of lipids and nucleic acid can be adjusted to provide a desired N/P ratio, for example, N/P ratio of 3, 4, 5, 6, 7, 8, 9, 10 or higher. Generally, the lipid nanoparticle formulation's overall lipid content can range from about 5 mg/ml to about 30 mg/mL.

Some non-limiting example of lipid compounds that may be used (e.g., in combination with other lipid components) to form lipid nanoparticles for the delivery of compositions described herein, e.g., nucleic acid (e.g., RNA (e.g., circular polyribonucleotide, linear polyribonucleotide)) described herein includes,

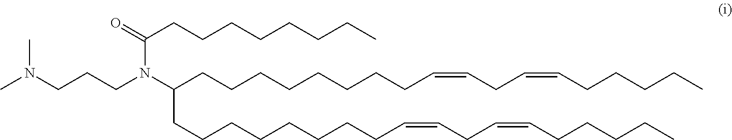 [SMILES: CCCCC/C=C\C/C=C\CCCCCCCCC(CCCCCCCC/C=C\C/C=C\CCCCC)N(CCCN(C)C)C(=O)CCCCCCCC]

In some embodiments an LNP including Formula (i) is used to deliver a polyribonucleotide (e.g., a circular polyribonucleotide, a linear polyribonucleotide) composition described herein to cells.

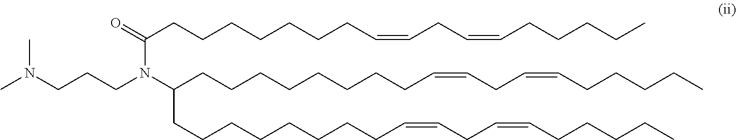 [SMILES: CCCCC/C=C\C/C=C\CCCCCCCCC(CCCCCCCC/C=C\C/C=C\CCCCC)N(CCCN(C)C)C(=O)CCCCCCC/C=C\C/C=C\CCCCC]

In some embodiments an LNP including Formula (ii) is used to deliver a polyribonucleotide (e.g., a circular polyribonucleotide, a linear polyribonucleotide) composition described herein to cells.

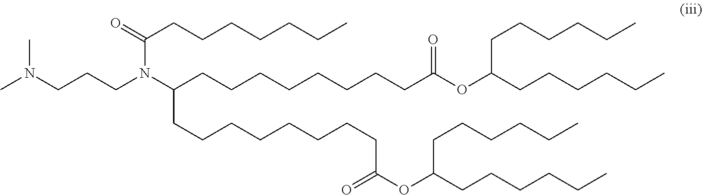 [SMILES: CCCCCCCC(=O)N(CCCN(C)C)C(CCCCCCCCC(=O)OC(CCCCCC)CCCCCC)CCCCCCCCC(=O)OC(CCCCCC)CCCCCC]

In some embodiments an LNP including Formula (iii) is used to deliver a polyribonucleotide (e.g., a circular polyribonucleotide, a linear polyribonucleotide) composition described herein to cells.

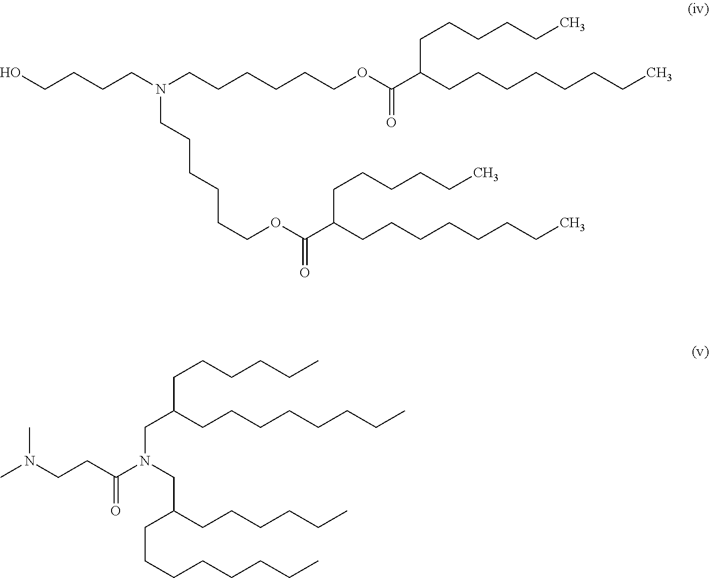

In some embodiments an LNP including Formula (v) is used to deliver a polyribonucleotide (e.g., a circular polyribonucleotide, a linear polyribonucleotide) composition described herein to cells.

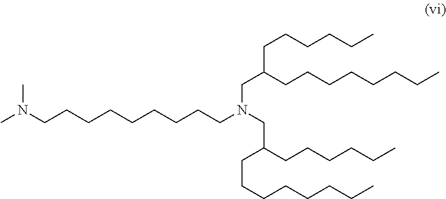 [SMILES: CCCCCCCCC(CCCCCC)CN(CCCCCCCCCN(C)C)CC(CCCCCC)CCCCCCCC]

In some embodiments an LNP including Formula (vi) is used to deliver a polyribonucleotide (e.g., a circular polyribonucleotide, a linear polyribonucleotide) composition described herein to cells.

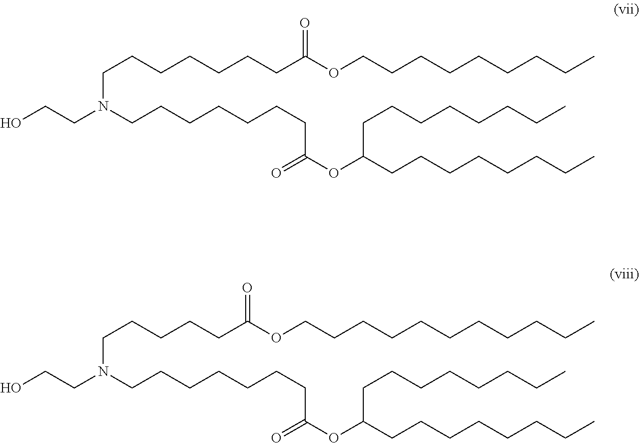 [SMILES: CCCCCCCCCOC(=O)CCCCCCCN(CCO)CCCCCCCC(=O)OC(CCCCCCCC)CCCCCCCC ; CCCCCCCCCCCOC(=O)CCCCCN(CCO)CCCCCCCC(=O)OC(CCCCCCCC)CCCCCCCC]

In some embodiments an LNP including Formula (viii) is used to deliver a polyribonucleotide (e.g., a circular polyribonucleotide, a linear polyribonucleotide) composition described herein to cells.

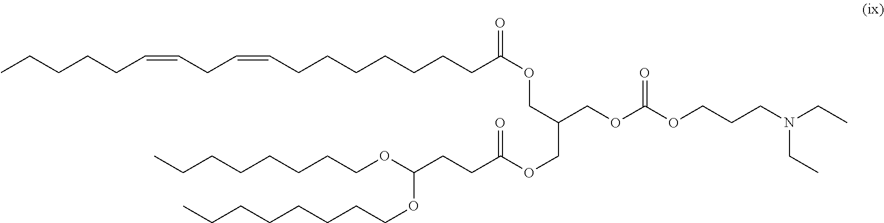 [SMILES: CCCCC/C=C\C/C=C\CCCCCCCC(=O)OCC(COC(=O)CCC(OCCCCCCCC)OCCCCCCCC)COC(=O)OCCCN(CC)CC]

In some embodiments an LNP including Formula (ix) is used to deliver a polyribonucleotide (e.g., a circular polyribonucleotide, a linear polyribonucleotide) composition described herein to cells.

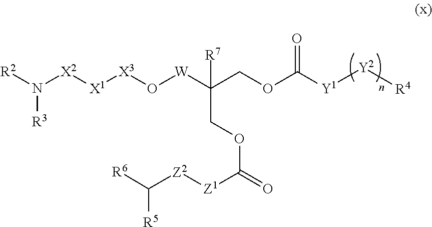

wherein
X¹ is O, NR¹, or a direct bond, X² is C₂₋₅ alkylene, X³ is C(═O) or a direct bond, R¹ is H or Me, R³ is C1-3 alkyl, R² is C1-3 alkyl, or R² taken together with the nitrogen atom to which it is attached and 1-3 carbon atoms of X² form a 4-, 5-, or 6-membered ring, or X¹ is NR¹, R¹ and R² taken together with the nitrogen atoms to which they are attached form a 5- or 6-membered ring, or R² taken together with R³ and the nitrogen atom to which they are attached form a 5-, 6-, or 7-membered ring, Y¹ is C2-12 alkylene, Y² is selected from

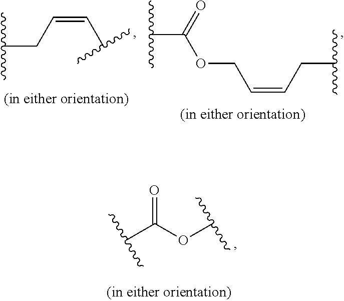 [SMILES: */C=C\C* ; *C/C=C\COC(*)=O ; *OC(*)=O]

n is 0 to 3, R⁴ is C1-15 alkyl, Z¹ is C1-6 alkylene or a direct bond,
Z² is

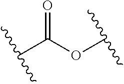 [SMILES: *OC(*)=O]

(in either orientation) or absent, provided that if Z¹ is a direct bond, Z² is absent;
R⁵ is C5-9 alkyl or C6-10 alkoxy, R⁶ is C5-9 alkyl or C6-10 alkoxy, W is methylene or a direct bond, and R⁷ is H or Me, or a salt thereof, provided that if R³ and R² are C2 alkyls, X¹ is O, X² is linear C₃ alkylene, X³ is C(═O), Y¹ is linear Ce alkylene, (Y²)ₙ-R⁴ is

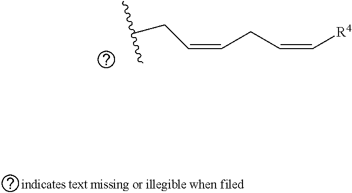

R⁴ is linear C5 alkyl, Z¹ is C2 alkylene, Z² is absent, W is methylene, and R⁷ is H, then R⁵ and R⁶ are not Cx alkoxy.

In some embodiments an LNP including Formula (xii) is used to deliver a polyribonucleotide (e.g., a circular polyribonucleotide, a linear polyribonucleotide) composition described herein to cells.

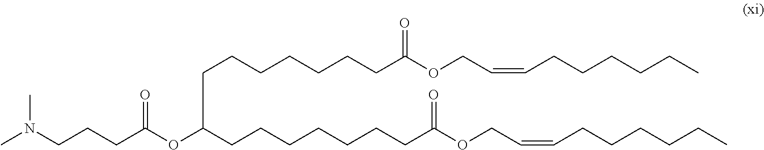 [SMILES: CCCCCC/C=C\COC(=O)CCCCCCCC(CCCCCCCC(=O)OC/C=C\CCCCCC)OC(=O)CCCN(C)C]

In some embodiments an LNP including Formula (xi) is used to deliver a polyribonucleotide (e.g., a circular polyribonucleotide, a linear polyribonucleotide) composition described herein to cells.

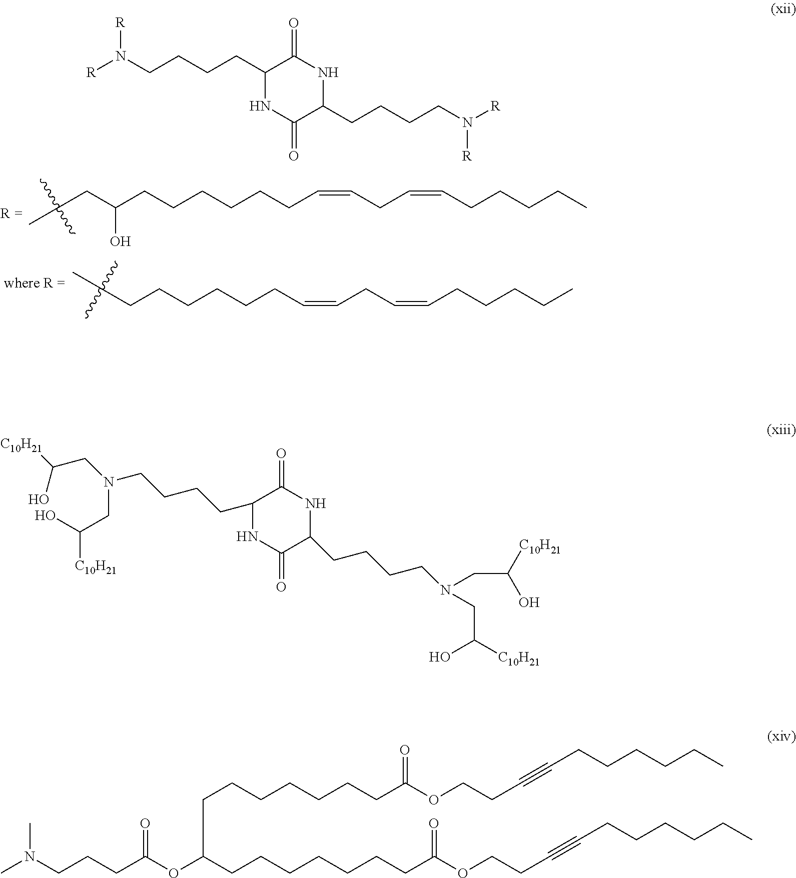

In some embodiments an LNP includes a compound of Formula (xiii) and a compound of Formula (xiv).

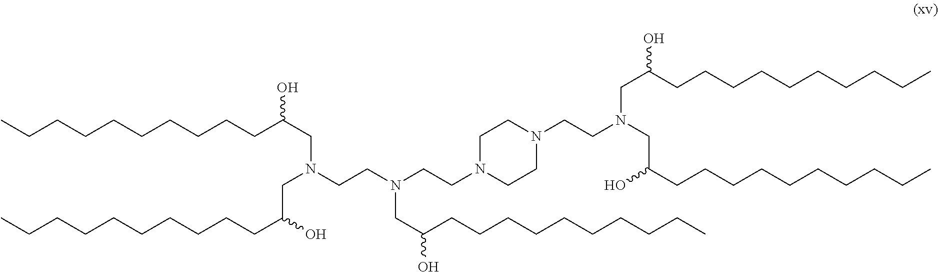 [SMILES: CCCCCCCCCCC(O)CN(CCN1CCN(CCN(CC(O)CCCCCCCCCC)CC(O)CCCCCCCCCC)CC1)CCN(CC(O)CCCCCCCCCC)CC(O)CCCCCCCCCC]

In some embodiments an LNP including Formula (xv) is used to deliver a polyribonucleotide (e.g., a circular polyribonucleotide, a linear polyribonucleotide) composition described herein to cells.

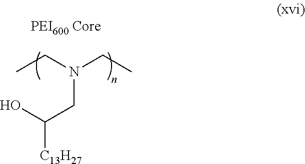

In some embodiments an LNP including a formulation of Formula (xvi) is used to deliver a polyribonucleotide (e.g., a circular polyribonucleotide, a linear polyribonucleotide) composition described herein to cells.

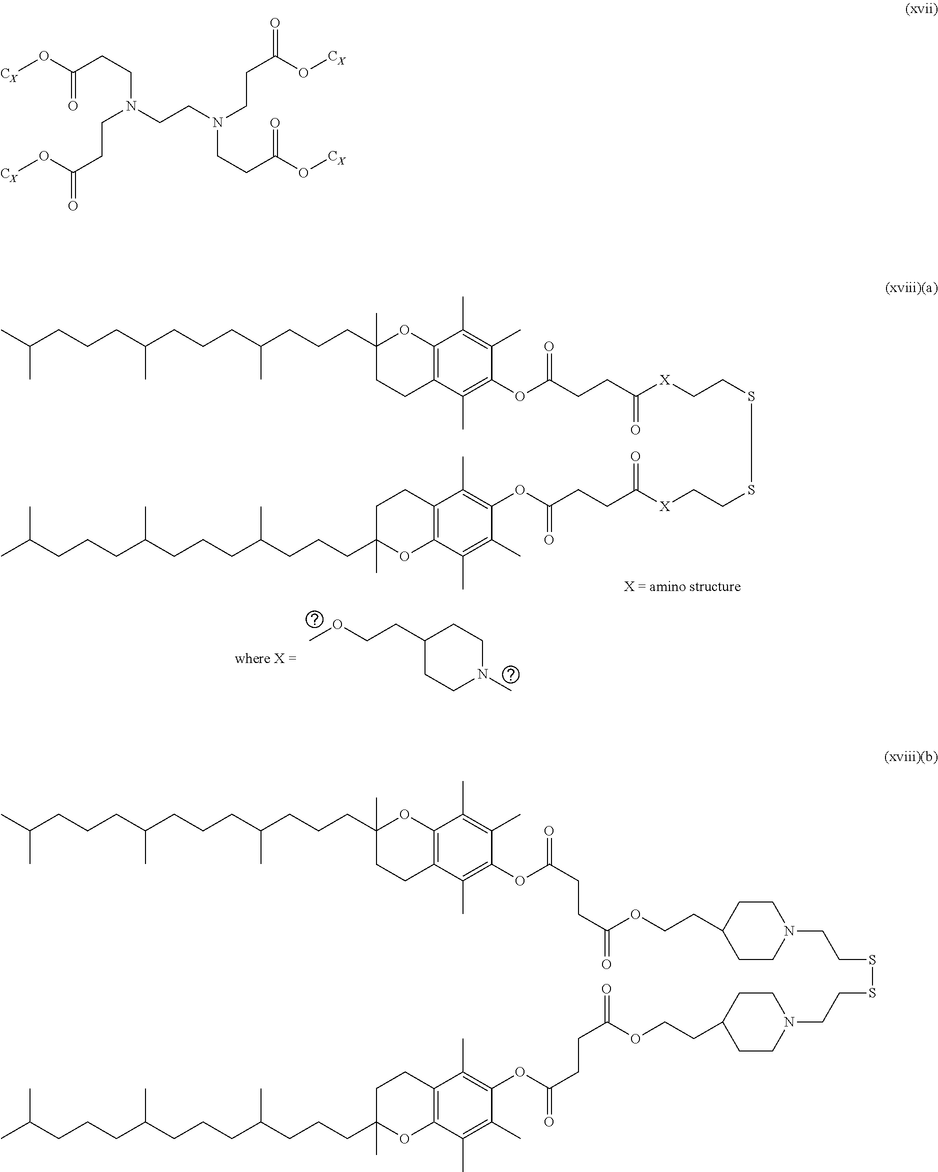

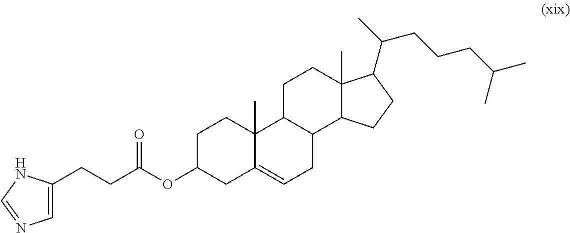 [SMILES: CC(C)CCCC(C)C1CCC2C3CC=C4CC(OC(=O)CCc5cnc[nH]5)CCC4(C)C3CCC12C]

In some embodiments, a lipid compound used to form lipid nanoparticles for the delivery of compositions described herein, e.g., nucleic acid (e.g., RNA (e.g., circular polyribonucleotide, linear polyribonucleotide)) described herein is made by one of the following reactions:

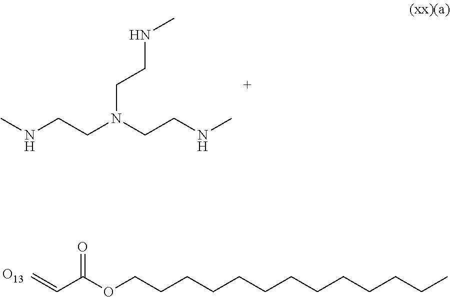

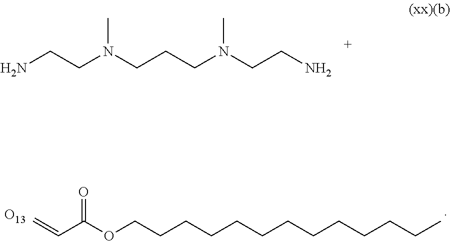 [SMILES: CN(CCN)CCCN(C)CCN ; "O13" ; C=CC(=O)OCCCCCCCCCCCCC]

In some embodiments an LNP including Formula (xxi) is used to deliver a polyribonucleotide (e.g., a circular polyribonucleotide, a linear polyribonucleotide) composition described herein to cells. In some embodiments the LNP of Formula (xxi) is an LNP described by WO2021113777 (e.g., a lipid of Formula (1) such as a lipid of Table 1 of WO2021113777).

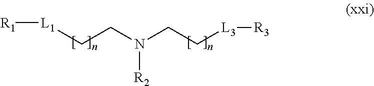

wherein
   each n is independently an integer from 2-15; L₁ and L₃ are each independently —OC(O)—* or —C(O)O—*, wherein “*” indicates the attachment point to R₁ or R₃;
   R₁ and R₃ are each independently a linear or branched C₉-C₂₀ alkyl or C₉-C₂₀ alkenyl, optionally substituted by one or more substituents selected from a group consisting of oxo, halo, hydroxy, cyano, alkyl, alkenyl, aldehyde, heterocyclylalkyl, hydroxyalkyl, dihydroxy alkyl, hydroxy alkyl amino alkyl, amino alkyl, alkylaminoalkyl, dialkylamino alkyl, (heterocyclyl)(alkyl)aminoalkyl, heterocyclyl, heteroaryl, alkyl heteroaryl, alkynyl, alkoxy, amino, dialkylamino, aminoalkylcarbonylamino, aminocarbonyl alkylamino, (aminocarbonylalkyl)(alkyl)amino, alkenylcarbonylamino, hydroxycarbonyl, alkyloxy carbonyl, aminocarbonyl, aminoalkylaminocarbonyl, alkylaminoalkylaminocarbonyl, dialkylaminoalkylaminocarbonyl, heterocyclylalkylaminocarbonyl, (alkylaminoalkyl)(alkyl)aminocarbonyl, alkylaminoalkylcarbonyl, dialkylaminoalkylcarbonyl, heterocyclylcarbonyl, alkenyl carbonyl, alkynyl carbonyl, alkyl sulfoxide, alkylsulfoxidealkyl, alkyl sulfonyl, and alkyl sulfone alkyl; and
   R₂ is selected from a group consisting of:

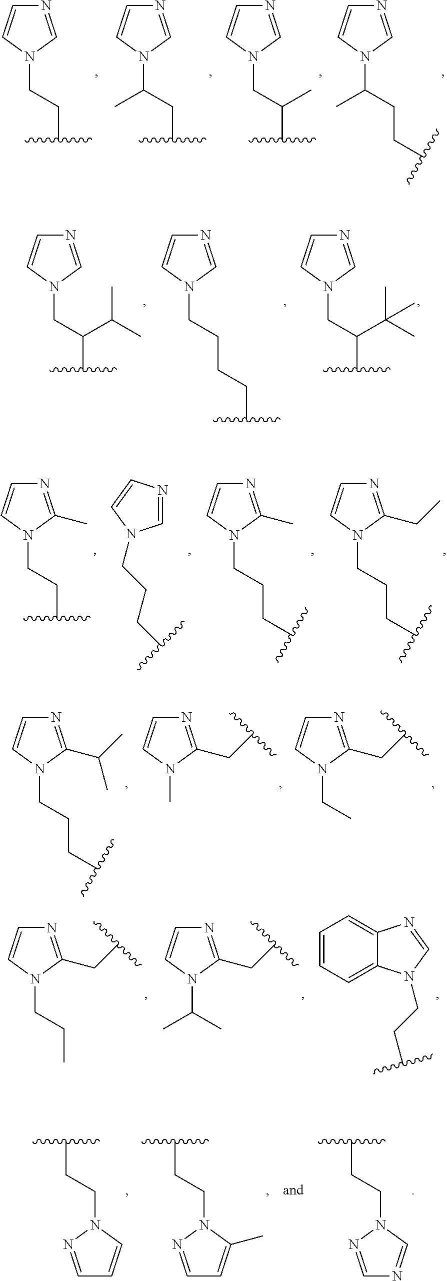

In some embodiments an LNP including Formula (xxii) is used to deliver a polyribonucleotide (e.g., a circular polyribonucleotide, a linear polyribonucleotide) composition described herein to cells. In some embodiments the LNP of Formula (xxii) is an LNP described by WO2021113777 (e.g., a lipid of Formula (2) such as a lipid of Table 2 of WO2021113777).

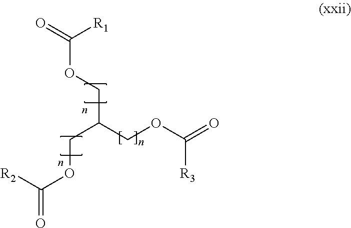

wherein
   each n is independently an integer from 1-15,
   R₁ and R₂ are each independently selected from a group consisting of:

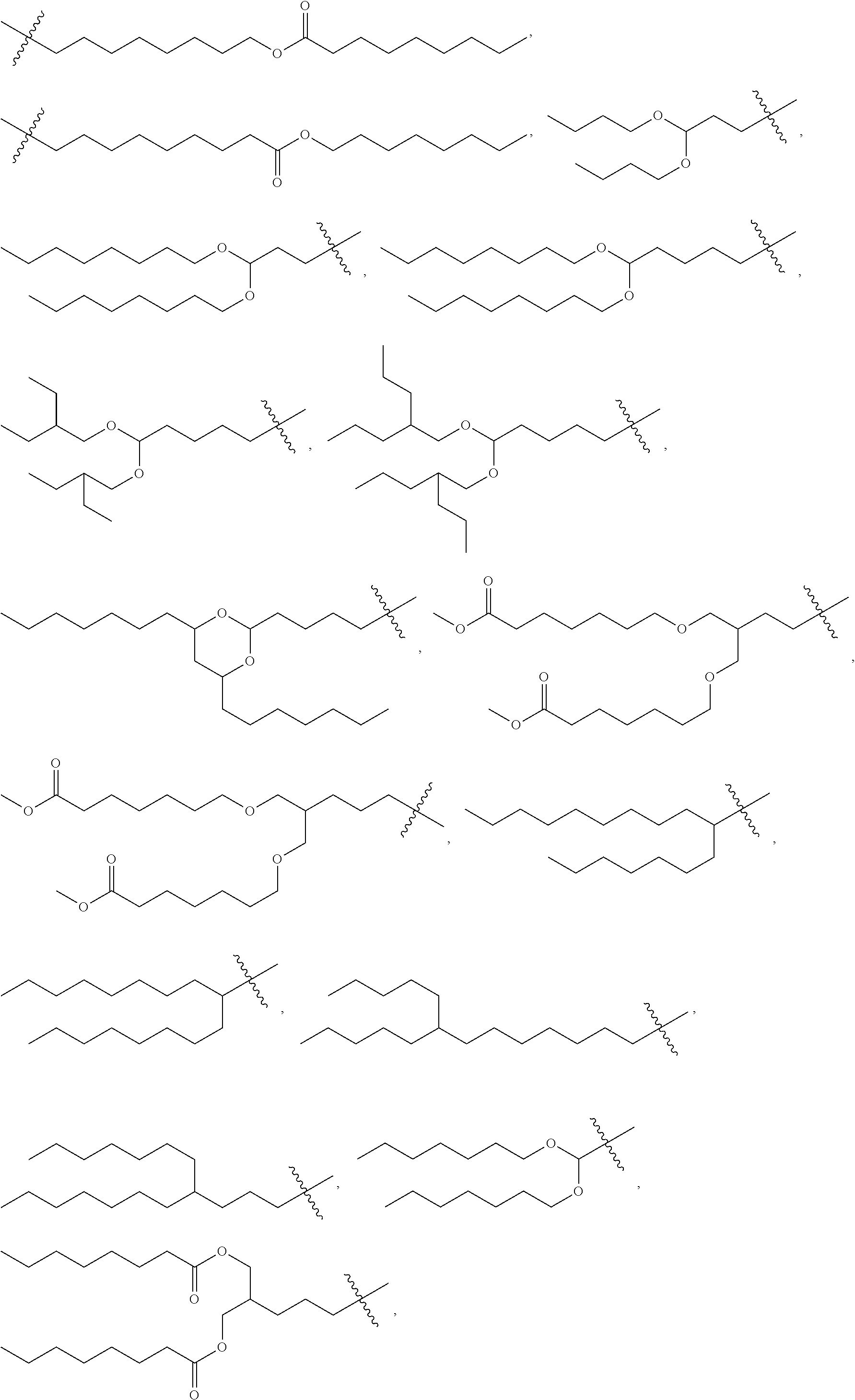

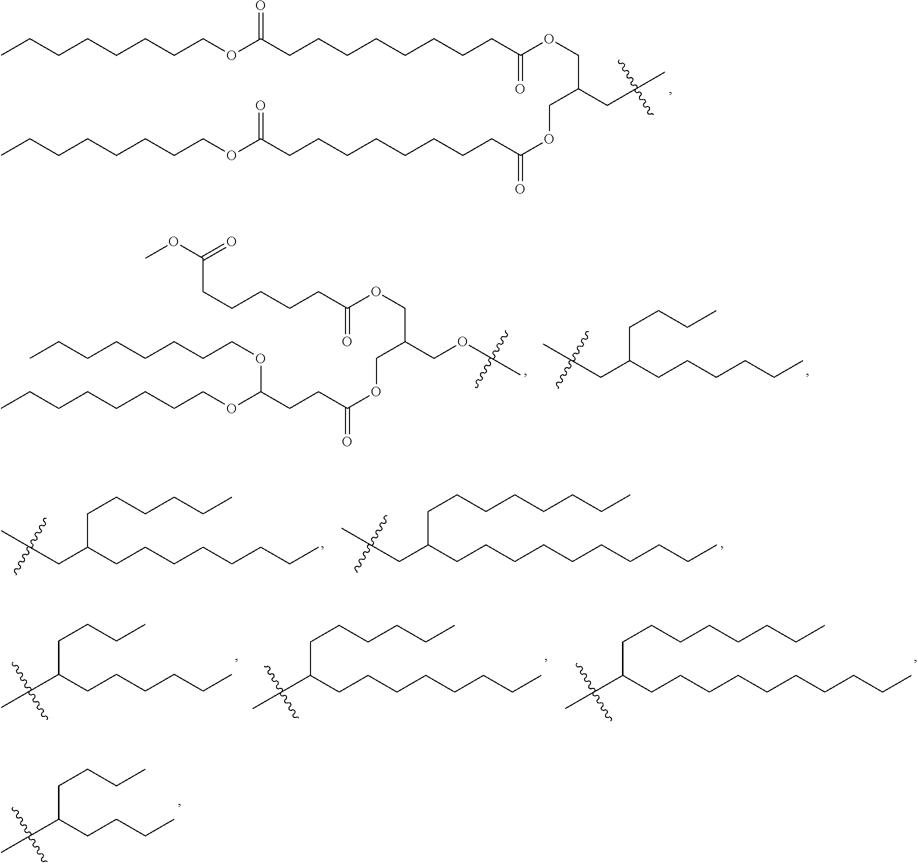

R₃ is selected from a group consisting of:

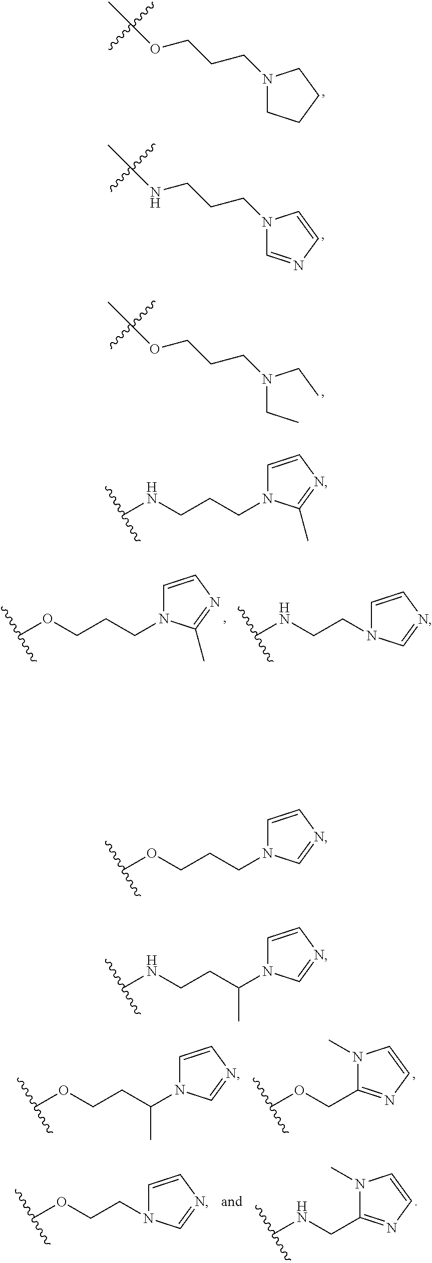

In some embodiments an LNP including Formula (xxiii) is used to deliver a polyribonucleotide (e.g., a circular polyribonucleotide, a linear polyribonucleotide) composition described herein to cells. In some embodiments the LNP of Formula (xxiii) is an LNP described by WO2021113777 (e.g., a lipid of Formula (3) such as a lipid of Table 3 of WO2021113777).

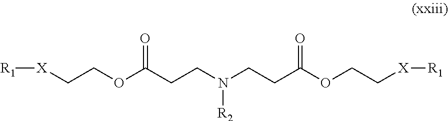

wherein
   X is selected from —O—, —S—, or —OC(O)—,* wherein indicates the attachment point to R₁;
   R₁ is selected rom a group consisting of:

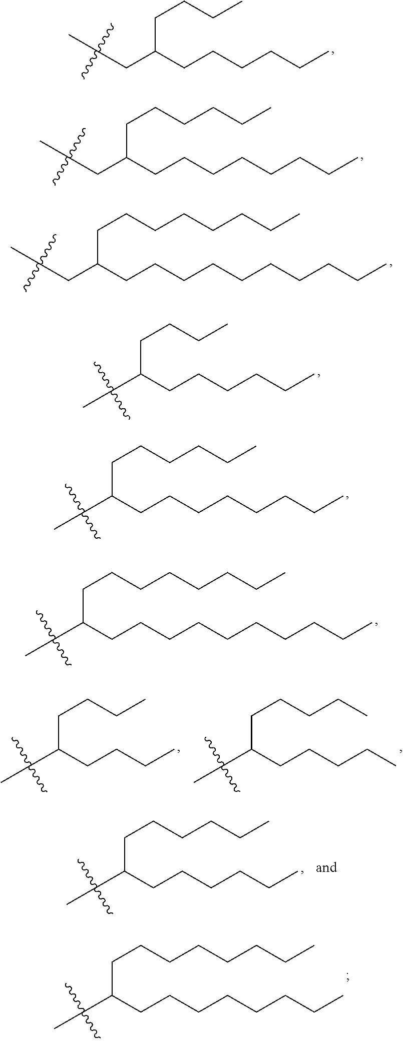

and R_{z} is selected from a group consisting of:

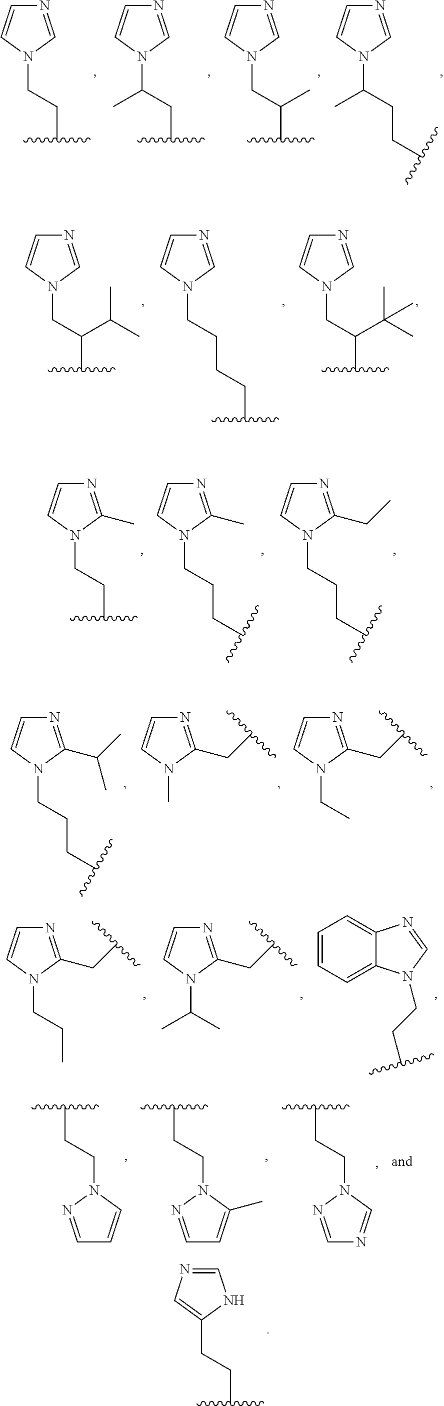

In some embodiments, a composition described herein (e.g., a nucleic acid (e.g., a circular polyribonucleotide, a linear polyribonucleotide) or a protein) is provided in an LNP that includes an ionizable lipid. In some embodiments, the ionizable lipid is heptadecan-9-yl 8-((2-hydroxyethyl) (6-oxo-6-(undecyloxy) hexyl)amino)octanoate (SM-102); e.g., as described in Example 1 of U.S. Pat. No. 9,867,888 (incorporated by reference herein in its entirety). In some embodiments, the ionizable lipid is 9Z,12Z)-3-((4,4-bis(octyloxy)butanoyl) oxy)-2-((((3-(diethylamino)propoxy)carbonyl)oxy)methyl)propyl octadeca-9,12-dienoate (LP01), e.g., as synthesized in Example 13 of WO2015/095340 (incorporated by reference herein in its entirety). In some embodiments, the ionizable lipid is Di((Z)-non-2-en-1-yl) 9-((4-dimethylamino)butanoyl)oxy)heptadecanedioate (L319), e.g., as synthesized in Example 7, 8, or 9 of US2012/0027803 (incorporated by reference herein in its entirety). In some embodiments, the ionizable lipid is 1,1′-((2-(4-(2-((2-(Bis(2-hydroxydodecyl)amino)ethyl)(2-hydroxydodecyl) amino)ethyl)piperazin-1-yl)ethyl)azanediyl)bis(dodecan-2-ol) (C12-200), e.g., as synthesized in Examples 14 and 16 of WO2010/053572 (incorporated by reference herein in its entirety). In some embodiments, the ionizable lipid is Imidazole cholesterol ester (ICE) lipid (3S,10R, 13R, 17R)-10, 13-dimethyl-17-((R)-6-methylheptan-2-yl)-2, 3, 4, 7, 8, 9, 10, 11, 12, 13, 14, 15, 16, 17-tetradecahydro-1H-cyclopenta[a]phenanthren-3-yl 3-(1H-imidazol-4-yl)propanoate, e.g., Structure (I) from WO2020/106946 (incorporated by reference herein in its entirety).

In some embodiments, an ionizable lipid may be a cationic lipid, an ionizable cationic lipid, e.g., a cationic lipid that can exist in a positively charged or neutral form depending on pH, or an amine-containing lipid that can be readily protonated. In some embodiments, the cationic lipid is a lipid capable of being positively charged, e.g., under physiological conditions. Exemplary cationic lipids include one or more amine group(s) which bear the positive charge. In some embodiments, the lipid particle includes a cationic lipid in formulation with one or more of neutral lipids, ionizable amine-containing lipids, biodegradable alkyne lipids, steroids, phospholipids including polyunsaturated lipids, structural lipids (e.g., sterols), PEG, cholesterol, and polymer conjugated lipids. In some embodiments, the cationic lipid may be an ionizable cationic lipid. An exemplary cationic lipid as disclosed herein may have an effective pKa over 6.0. In embodiments, a lipid nanoparticle may include a second cationic lipid having a different effective pKa (e.g., greater than the first effective pKa), than the first cationic lipid. A lipid nanoparticle may include between 40 and 60 mol percent of a cationic lipid, a neutral lipid, a steroid, a polymer conjugated lipid, and a therapeutic agent, e.g., a nucleic acid (e.g., RNA (e.g., a circular polyribonucleotide, a linear polyribonucleotide)) described herein, encapsulated within or associated with the lipid nanoparticle. In some embodiments, the nucleic acid is co-formulated with the cationic lipid. The nucleic acid may be adsorbed to the surface of an LNP, e.g., an LNP including a cationic lipid. In some embodiments, the nucleic acid may be encapsulated in an LNP, e.g., an LNP including a cationic lipid. In some embodiments, the lipid nanoparticle may include a targeting moiety, e.g., coated with a targeting agent. In embodiments, the LNP formulation is biodegradable. In some embodiments, a lipid nanoparticle including one or more lipid described herein, e.g., Formula (i), (ii), (ii), (vii) and/or (ix) encapsulates at least 1%, at least 5%, at least 10%, at least 20%, at least 30%, at least 40%, at least 50%, at least 60%, at least 70%, at least 80%, at least 90%, at least 92%, at least 95%, at least 97%, at least 98% or 100% of an RNA molecule.

Exemplary ionizable lipids that can be used in lipid nanoparticle formulations include, without limitation, those listed in Table 1 of WO2019051289, incorporated herein by reference. Additional exemplary lipids include, without limitation, one or more of the following formulae: X of US2016/0311759; I of US20150376115 or in US2016/0376224; 1, 11 or III of US20160151284; I, IA, II, or IIA of US20170210967; I-c of US20150140070; A of US2013/0178541; I of US2013/0303587 or US2013/0123338; I of US2015/0141678; II, III, IV, or V of US2015/0239926; I of US2017/0119904; I or II of WO2017/117528; A of US2012/0149894; A of US2015/0057373; A of WO2013/116126; A of US2013/0090372; A of US2013/0274523; A of US2013/0274504; A of US2013/0053572; A of WO2013/016058; A of WO2012/162210; I of US2008/042973; I, II, III, or IV of US2012/01287670; I or II of US2014/0200257; 1, 11, or III of US2015/0203446; I or III of US2015/0005363; I, IA, IB, IC, ID, II, IIA, IIB, IIC, IID, or III-XXIV of US2014/0308304; of US2013/0338210; 1, 11, 111, or IV of WO2009/132131; A of US2012/01011478; I or XXXV of US2012/0027796; XIV or XVII of US2012/0058144; of US2013/0323269; I of US2011/0117125; 1, 11, or III of US2011/0256175; 1, 11, 111, IV, V, VI, VII, VIII, IX, X, XI, XII of US2012/0202871; 1, 11, 111, IV, V, VI, VII, VIII, X, XII, XIII, XIV, XV, or XVI of US2011/0076335; I or II of US2006/008378; I of US2013/0123338; I or X-A-Y-Z of US2015/0064242; XVI, XVII, or XVIII of US2013/0022649; 1, or Ill of US2013/0116307; 1, or Ill of US2013/0116307; I or II of US2010/0062967; I-X of US2013/0189351; I of US2014/0039032; V of US2018/0028664; I of US2016/0317458; I of US2013/0195920; 5, 6, or 10 of U.S. Pat. No. 10,221,127; 111-3 of WO2018/081480; 1-5 or 1-8 of WO2020/081938; 18 or 25 of U.S. Pat. No. 9,867,888; A of US2019/0136231; II of WO2020/219876; 1 of US2012/0027803; OF-02 of US2019/0240349; 23 of U.S. Pat. No. 10,086,013; cKK-E12/A6 of Miao et al (2020); C12-200 of WO2010/053572; 7C1 of Dahlman et al (2017); 304-013 or 503-013 of Whitehead et al; TS-P4C2 of U.S. Pat. No. 9,708,628; I of WO2020/106946; I of WO2020/106946; and (1), (2), (3), or (4) of WO2021/113777. Exemplary lipids further include a lipid of any one of Tables 1-16 of WO2021/113777.

In some embodiments, the ionizable lipid is MC3 (6Z,9Z,28Z,3 IZ)-heptatriaconta-6,9,28,31-tetraen-19-yl-4-(dimethylamino) butanoate (DLin-MC3-DMA or MC3), e.g., as described in Example 9 of WO2019051289A9 (incorporated by reference herein in its entirety). In some embodiments, the ionizable lipid is the lipid ATX-002, e.g., as described in Example 10 of WO2019051289A9 (incorporated by reference herein in its entirety). In some embodiments, the ionizable lipid is (13Z,16Z)-A,A-dimethyl-3-nonyldocosa-13, 16-dien-1-amine (Compound 32), e.g., as described in Example 11 of WO2019051289A9 (incorporated by reference herein in its entirety). In some embodiments, the ionizable lipid is Compound 6 or Compound 22, e.g., as described in Example 12 of WO2019051289A9 (incorporated by reference herein in its entirety).

Exemplary non-cationic lipids include, but are not limited to, distearoyl-sn-glycero-phosphoethanolamine, distearoylphosphatidylcholine (DSPC), dioleoyl phosphatidylcholine (DOPC), dipalmitoylphosphatidylcholine (DPPC), dioleoyl phosphatidylglycerol (DOPG), dipalmitoylphosphatidylglycerol (DPPG), dioleoyl-phosphatidylethanolamine (DOPE), palmitoyloleoylphosphatidylcholine (POPC), palmitoyloleoylphosphatidylethanolamine (POPE), dioleoyl-phosphatidylethanolamine 4-(N-maleimidomethyl)-cyclohexane-1-carboxylate (DOPE-mal), dipalmitoyl phosphatidyl ethanolamine (DPPE), dimyristoylphosphoethanolamine (DMPE), distearoyl-phosphatidyl-ethanolamine (DSPE), monomethyl-phosphatidylethanolamine (such as 16-O-monomethyl PE), dimethyl-phosphatidylethanolamine (such as 16-O-dimethyl PE), 18-1-trans PE, I-stearoyl-2-oleoyl-phosphatidylethanolamine (SOPE), hydrogenated soy phosphatidylcholine (HSPC), egg phosphatidylcholine (EPC), dioleoyl phosphatidylserine (DOPS), sphingomyelin (SM), dimyristoyl phosphatidylcholine (DMPC), dimyristoyl phosphatidylglycerol (DMPG), distearoylphosphatidylglycerol (DSPG), dierucoylphosphatidylcholine (DEPC), palmitoyloleyolphosphatidylglycerol (POPG), dielaidoyl-phosphatidylethanolamine (DEPE), lecithin, phosphatidylethanolamine, lysolecithin, Lys phosphatidylethanolamine, phosphatidylserine, phosphatidylinositol, sphingomyelin, egg sphingomyelin (ESM), cephalin, cardiolipin, phosphatidic acid, cerebrosides, dicetylphosphate, Lys phosphatidylcholine, dilinoleoylphosphatidylcholine, or mixtures thereof. It is understood that other diacyl phosphatidylcholine and diacyl phosphatidylethanolamine phospholipids can also be used. The acyl groups in these lipids are preferably acyl groups derived from fatty acids having C10-C24 carbon chains, e.g., lauroyl, myristoyl, paimitoyl, stearoyl, or oleoyl. Additional exemplary lipids, in certain embodiments, include, without limitation, those described in Kim et al. (2020) dx.doi.org/10.1021/acs.nanolett.0c01386, incorporated herein by reference. Such lipids include, in some embodiments, plant lipids found to improve liver transfection with mRNA (e.g., DGTS).

Other examples of non-cationic lipids suitable for use in the lipid nanoparticles include, without limitation, non-phosphorous lipids such as, e.g., stearylamine, dodeeylamine, hexadecyl amine, acetyl palmitate, glycerol ricin oleate, hexadecyl stearate, isopropyl myristate, amphoteric acrylic polymers, triethanolamine-lauryl sulfate, alkyl-aryl sulfate polyethyloxylated fatty acid amides, dioctadecyl dimethyl ammonium bromide, ceramide, sphingomyelin, and the like. Other non-cationic lipids are described in WO2017/099823 or US patent publication US2018/0028664, the contents of which is incorporated herein by reference in their entirety.

In some embodiments, the non-cationic lipid is oleic acid or a compound of Formula I, II, or IV of US2018/0028664, incorporated herein by reference in its entirety. The non-cationic lipid can include, for example, 0-30% (mol) of the total lipid present in the lipid nanoparticle. In some embodiments, the non-cationic lipid content is 5-20% (mol) or 10-15% (mol) of the total lipid present in the lipid nanoparticle. In embodiments, the molar ratio of ionizable lipid to the neutral lipid ranges from about 2:1 to about 8:1 (e.g., about 2:1, 3:1, 4:1, 5:1, 6:1, 7:1, or 8:1).

In some embodiments, the lipid nanoparticles do not include any phospholipids.

In some aspects, the lipid nanoparticle can further include a component, such as a sterol, to provide membrane integrity. One exemplary sterol that can be used in the lipid nanoparticle is cholesterol and derivatives thereof. Non-limiting examples of cholesterol derivatives include polar analogues such as 5a-cholestanol, 53-coprostanol, cholesteryl-(2,-hydroxy)-ethyl ether, cholesteryl-(4′-hydroxy)-butyl ether, and 6-ketocholestanol; non-polar analogues such as 5a-cholestane, cholestenone, 5a-cholestanone, 5p-cholestanone, and cholesteryl decanoate; and mixtures thereof. In some embodiments, the cholesterol derivative is a polar analogue, e.g., cholesteryl-(4 ′-hydroxy)-butyl ether. Exemplary cholesterol derivatives are described in PCT Publication No. WO2009/127060 and US patent publication US2010/0130588, each of which is incorporated herein by reference in its entirety.

In some embodiments, the component providing membrane integrity, such as a sterol, can include 0-50% (mol) (e.g., 0-10%, 10-20%, 20-30%, 30-40%, or 40-50%) of the total lipid present in the lipid nanoparticle. In some embodiments, such a component is 20-50% (mol) 30-40% (mol) of the total lipid content of the lipid nanoparticle.

In some embodiments, the lipid nanoparticle can include a polyethylene glycol (PEG) or a conjugated lipid molecule. Generally, these are used to inhibit aggregation of lipid nanoparticles and/or provide steric stabilization. Exemplary conjugated lipids include, but are not limited to, PEG-lipid conjugates, polyoxazoline (POZ)-lipid conjugates, polyamide-lipid conjugates (such as ATTA-lipid conjugates), cationic-polymer lipid (CPL) conjugates, and mixtures thereof. In some embodiments, the conjugated lipid molecule is a PEG-lipid conjugate, for example, a (methoxy polyethylene glycol)-conjugated lipid.

Exemplary PEG-lipid conjugates include, but are not limited to, PEG-diacylglycerol (DAG) (such as 1-(monomethoxy-polyethylene glycol)-2,3-dimyristoylglycerol (PEG-DMG)), PEG-dialkyloxypropyl (DAA), PEG-phospholipid, PEG-ceramide (Cer), a pegylated phosphatidylethanoloamine (PEG-PE), PEG succinate diacylglycerol (PEGS-DAG) (such as 4-0-(2′,3′-di(tetradecanoyloxy)propyl-1-O-(w-methoxy(polyethoxy)ethyl) butanedioate (PEG-S-DMG)), PEG dialkoxypropylcarbam, N-(carbonyl-methoxypolyethylene glycol 2000)-1,2-distearoyl-sn-glycero-3-phosphoethanolamine sodium salt, or a mixture thereof. Additional exemplary PEG-lipid conjugates are described, for example, in U.S. Pat. Nos. 5,885,613, 6,287,591, US2003/0077829, US2003/0077829, US2005/0175682, US2008/0020058, US2011/0117125, US2010/0130588, US2016/0376224, US2017/0119904, US2018/0028664, and WO2017/099823, the contents of all of which are incorporated herein by reference in their entirety. In some embodiments, a PEG-lipid is a compound of Formula III, III-a-1, III-a-2, Ill-b-1, Ill-b-2, or V of US2018/0028664, the content of which is incorporated herein by reference in its entirety. In some embodiments, a PEG-lipid is of Formula II of US20150376115 or US2016/0376224, the content of both of which is incorporated herein by reference in its entirety. In some embodiments, the PEG-DAA conjugate can be, for example, PEG-dilauryloxypropyl, PEG-dimyristyloxypropyl, PEG-dipalmityloxypropyl, or PEG-distearyloxypropyl. The PEG-lipid can be one or more of PEG-DMG, PEG-dilaurylglycerol, PEG-dipalmitoylglycerol, PEG-disterylglycerol, PEG-dilaurylglycamide, PEG-dimyristylglycamide, PEG-dipalmitoylglycamide, PEG-disterylglycamide, PEG-cholesterol (I-[8′-(Cholest-5-en-3[beta]-oxy)carboxamido-3′,6′-dioxaoctanyl]carbamoyl-[omega]-methyl-poly(ethylene glycol), PEG-DMB (3,4-Ditetradecoxylbenzyl-[omega]-methyl-poly(ethylene glycol) ether), and 1,2-dimyristoyl-sn-glycero-3-phosphoethanolamine-N-[methoxy(polyethylene glycol)-2000]. In some embodiments, the PEG-lipid includes PEG-DMG, 1,2-dimyristoyl-sn-glycero-3-phosphoethanolamine-N-[methoxy(polyethylene glycol)-2000]. In some embodiments, the PEG-lipid includes a structure selected from:

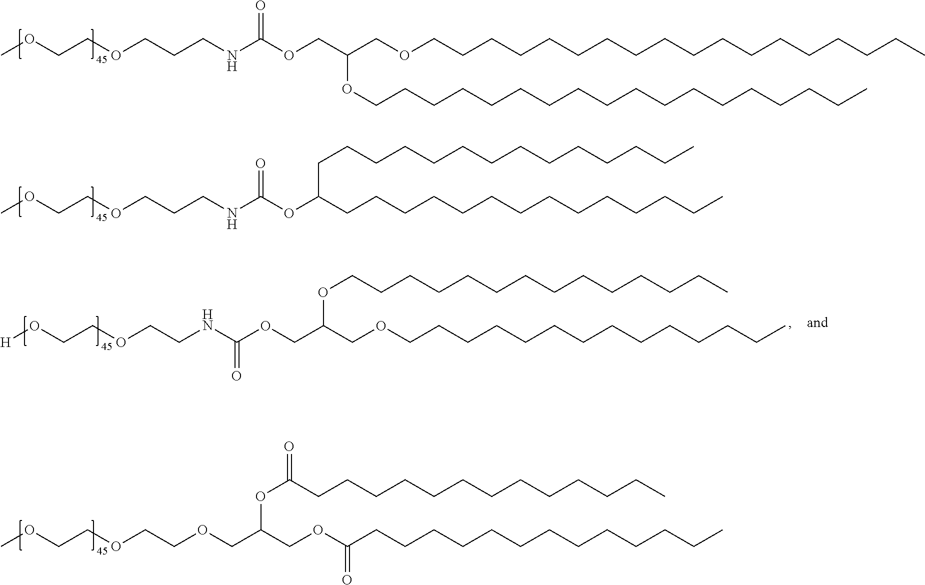

In some embodiments, lipids conjugated with a molecule other than a PEG can also be used in place of PEG-lipid. For example, polyoxazoline (POZ)-lipid conjugates, polyamide-lipid conjugates (such as ATTA-lipid conjugates), and cationic-polymer lipid (GPL) conjugates can be used in place of or in addition to the PEG-lipid.

Exemplary conjugated lipids, i.e., PEG-lipids, (POZ)-lipid conjugates, ATTA-lipid conjugates and cationic polymer-lipids are described in the PCT, and LIS patent applications listed in Table 2 of WO2019051289A9, the contents of all of which are incorporated herein by reference in their entirety.

In some embodiments, the PEG or the conjugated lipid can include 0-20% (mol) of the total lipid present in the lipid nanoparticle. In some embodiments, PEG or the conjugated lipid content is 0.5-10% or 2-5% (mol) of the total lipid present in the lipid nanoparticle. Molar ratios of the ionizable lipid, non-cationic-lipid, sterol, and PEG/conjugated lipid can be varied as needed. For example, the lipid particle can include 30-70% ionizable lipid by mole or by total weight of the composition, 0-60% cholesterol by mole or by total weight of the composition, 0-30% non-cationic lipid by mole or by total weight of the composition and 1-10% conjugated lipid by mole or by total weight of the composition. Preferably, the composition includes 30-40% ionizable lipid by mole or by total weight of the composition, 40-50% cholesterol by mole or by total weight of the composition, and 10-20% non-cationic-lipid by mole or by total weight of the composition. In some other embodiments, the composition is 50-75% ionizable lipid by mole or by total weight of the composition, 20-40% cholesterol by mole or by total weight of the composition, and 5 to 10% non-cationic lipid, by mole or by total weight of the composition and 1-10% conjugated lipid by mole or by total weight of the composition. The composition may contain 60-70% ionizable lipid by mole or by total weight of the composition, 25-35% cholesterol by mole or by total weight of the composition, and 5-10% non-cationic lipid by mole or by total weight of the composition. The composition may also contain up to 90% ionizable lipid by mole or by total weight of the composition and 2 to 15% non-cationic lipid by mole or by total weight of the composition. The formulation may also be a lipid nanoparticle formulation, for example including 8-30% ionizable lipid by mole or by total weight of the composition, 5-30% non-cationic lipid by mole or by total weight of the composition, and 0-20% cholesterol by mole or by total weight of the composition; 4-25% ionizable lipid by mole or by total weight of the composition, 4-25% non-cationic lipid by mole or by total weight of the composition, 2 to 25% cholesterol by mole or by total weight of the composition, 10 to 35% conjugate lipid by mole or by total weight of the composition, and 5% cholesterol by mole or by total weight of the composition; or 2-30% ionizable lipid by mole or by total weight of the composition, 2-30% non-cationic lipid by mole or by total weight of the composition, 1 to 15% cholesterol by mole or by total weight of the composition, 2 to 35% conjugate lipid by mole or by total weight of the composition, and 1-20% cholesterol by mole or by total weight of the composition; or even up to 90% ionizable lipid by mole or by total weight of the composition and 2-10% non-cationic lipids by mole or by total weight of the composition, or even 100% cationic lipid by mole or by total weight of the composition. In some embodiments, the lipid particle formulation includes ionizable lipid, phospholipid, cholesterol and a PEG-ylated lipid in a molar ratio of 50:10:38.5:1.5. In some other embodiments, the lipid particle formulation includes ionizable lipid, cholesterol and a PEG-ylated lipid in a molar ratio of 60:38.5:1.5.

In some embodiments, the lipid particle includes ionizable lipid, non-cationic lipid (e.g., phospholipid), a sterol (e.g., cholesterol) and a PEG-ylated lipid, where the molar ratio of lipids ranges from 20 to 70 mole percent for the ionizable lipid, with a target of 40-60, the mole percent of non-cationic lipid ranges from 0 to 30, with a target of 0 to 15, the mole percent of sterol ranges from 20 to 70, with a target of 30 to 50, and the mole percent of PEG-ylated lipid ranges from 1 to 6, with a target of 2 to 5.

In some embodiments, the lipid particle includes ionizable lipid/non-cationic-lipid/sterol/conjugated lipid at a molar ratio of 50:10:38.5:1.5.

In an aspect, the disclosure provides a lipid nanoparticle formulation including phospholipids, lecithin, phosphatidylcholine and phosphatidylethanolamine.

In some embodiments, one or more additional compounds can also be included. Those compounds can be administered separately, or the additional compounds can be included in the lipid nanoparticles of the invention. In other words, the lipid nanoparticles can contain other compounds in addition to the nucleic acid or at least a second nucleic acid, different than the first. Without limitations, other additional compounds can be selected from the group consisting of small or large organic or inorganic molecules, monosaccharides, disaccharides, trisaccharides, oligosaccharides, polysaccharides, peptides, proteins, peptide analogs and derivatives thereof, peptidomimetics, nucleic acids, nucleic acid analogs and derivatives, an extract made from biological materials, or any combinations thereof.

In some embodiments, the LNPs include biodegradable, ionizable lipids. In some embodiments, the LNPs include (9Z,12Z)-3-((4,4-bis(octyloxy)butanoyl)oxy)-2-((((3-(diethylamino)propoxy)carbonyl)oxy)methyl)propyl octadeca-9,12-dienoate, also called 3-((4,4-bis(octyloxy)butanoyl)oxy)-2-((((3-(diethylamino)propoxy)carbonyl)oxy)methyl)propyl (9Z,12Z)-octadeca-9,12-dienoate) or another ionizable lipid. See, e.g., lipids of WO2019/067992, WO/2017/173054, WO2015/095340, and WO2014/136086, as well as references provided therein. In some embodiments, the term cationic and ionizable in the context of LNP lipids is interchangeable, e.g., wherein ionizable lipids are cationic depending on the pH.

In some embodiments, the average LNP diameter of the LNP formulation may be between 10s of nm and 100s of nm, e.g., measured by dynamic light scattering (DLS). In some embodiments, the average LNP diameter of the LNP formulation may be from about 40 nm to about 150 nm, such as about 40 nm, 45 nm, 50 nm, 55 nm, 60 nm, 65 nm, 70 nm, 75 nm, 80 nm, 85 nm, 90 nm, 95 nm, 100 nm, 105 nm, 110 nm, 115 nm, 120 nm, 125 nm, 130 nm, 135 nm, 140 nm, 145 nm, or 150 nm. In some embodiments, the average LNP diameter of the LNP formulation may be from about 50 nm to about 100 nm, from about 50 nm to about 90 nm, from about 50 nm to about 80 nm, from about 50 nm to about 70 nm, from about 50 nm to about 60 nm, from about 60 nm to about 100 nm, from about 60 nm to about 90 nm, from about 60 nm to about 80 nm, from about 60 nm to about 70 nm, from about 70 nm to about 100 nm, from about 70 nm to about 90 nm, from about 70 nm to about 80 nm, from about 80 nm to about 100 nm, from about 80 nm to about 90 nm, or from about 90 nm to about 100 nm. In some embodiments, the average LNP diameter of the LNP formulation may be from about 70 nm to about 100 nm. In a particular embodiment, the average LNP diameter of the LNP formulation may be about 80 nm. In some embodiments, the average LNP diameter of the LNP formulation may be about 100 nm. In some embodiments, the average LNP diameter of the LNP formulation ranges from about 1 mm to about 500 mm, from about 5 mm to about 200 mm, from about 10 mm to about 100 mm, from about 20 mm to about 80 mm, from about 25 mm to about 60 mm, from about 30 mm to about 55 mm, from about 35 mm to about 50 mm, or from about 38 mm to about 42 mm.

A LNP may, in some instances, be relatively homogenous. A polydispersity index may be used to indicate the homogeneity of a LNP, e.g., the particle size distribution of the lipid nanoparticles. A small (e.g., less than 0.3) polydispersity index generally indicates a narrow particle size distribution. A LNP may have a polydispersity index from about 0 to about 0.25, such as 0.01, 0.02, 0.03, 0.04, 0.05, 0.06, 0.07, 0.08, 0.09, 0.10, 0.11, 0.12, 0.13, 0.14, 0.15, 0.16, 0.17, 0.18, 0.19, 0.20, 0.21, 0.22, 0.23, 0.24, or 0.25. In some embodiments, the polydispersity index of a LNP may be from about 0.10 to about 0.20.

The zeta potential of a LNP may be used to indicate the electrokinetic potential of the composition. In some embodiments, the zeta potential may describe the surface charge of an LNP. Lipid nanoparticles with relatively low charges, positive or negative, are generally desirable, as more highly charged species may interact undesirably with cells, tissues, and other elements in the body. In some embodiments, the zeta potential of a LNP may be from about −10 mV to about +20 mV, from about −10 mV to about +15 mV, from about −10 mV to about +10 mV, from about −10 mV to about +5 mV, from about −10 mV to about 0 mV, from about −10 mV to about −5 mV, from about −5 mV to about +20 mV, from about −5 mV to about +15 mV, from about −5 mV to about +10 mV, from about −5 mV to about +5 mV, from about −5 mV to about 0 mV, from about 0 mV to about +20 mV, from about 0 mV to about +15 mV, from about 0 mV to about +10 mV, from about 0 mV to about +5 mV, from about +5 mV to about +20 mV, from about +5 mV to about +15 mV, or from about +5 mV to about +10 mV.

The efficiency of encapsulation of a protein and/or nucleic acid, describes the amount of protein and/or nucleic acid that is encapsulated or otherwise associated with a LNP after preparation, relative to the initial amount provided. The encapsulation efficiency is desirably high (e.g., close to 100%). The encapsulation efficiency may be measured, for example, by comparing the amount of protein or nucleic acid in a solution containing the lipid nanoparticle before and after breaking up the lipid nanoparticle with one or more organic solvents or detergents. An anion exchange resin may be used to measure the amount of free protein or nucleic acid (e.g., RNA) in a solution. Fluorescence may be used to measure the amount of free protein and/or nucleic acid (e.g., RNA) in a solution. For the lipid nanoparticles described herein, the encapsulation efficiency of a protein and/or nucleic acid may be at least 50%, for example 50%, 55%, 60%, 65%, 70%, 75%, 80%, 85%, 90%, 91%, 92%, 93%, 94%, 95%, 96%, 97%, 98%, 99%, or 100%. In some embodiments, the encapsulation efficiency may be at least 80%. In some embodiments, the encapsulation efficiency may be at least 90%. In some embodiments, the encapsulation efficiency may be at least 95%.

A LNP may optionally include one or more coatings. In some embodiments, a LNP may be formulated in a capsule, film, or table having a coating. A capsule, film, or tablet including a composition described herein may have any useful size, tensile strength, hardness or density.

Additional exemplary lipids, formulations, methods, and characterization of LNPs are taught by WO2020/061457 and WO2021/113777, each of which is incorporated herein by reference in its entirety. Further exemplary lipids, formulations, methods, and characterization of LNPs are taught by Hou et al. Lipid nanoparticles for mRNA delivery. Nat Rev Mater (2021). doi.org/10.1038/s41578-021-00358-0, which is incorporated herein by reference in its entirety (see, for example, exemplary lipids and lipid derivatives of FIG. 2 of Hou et al.).

In some embodiments, in vitro or ex vivo cell lipofections are performed using LIPOFECTAMINE® MessengerMax (Thermo Fisher) or TransIT-mRNA Transfection Reagent (Mirus Bio). In certain embodiments, LNPs are formulated using the GenVoy_ILM ionizable lipid mix (Precision NanoSystems). In certain embodiments, LNPs are formulated using 2,2-dilinoleyl-4-dimethylaminoethyl-[1,3]-dioxolane (DLin-KC2-DMA) or dilinoleylmethyl-4-dimethylaminobutyrate (DLin-MC3-DMA or MC3), the formulation and in vivo use of which are taught in Jayaraman et al. Angew Chem Int Ed Engl 51(34):8529-8533 (2012), incorporated herein by reference in its entirety.

LNP formulations optimized for the delivery of CRISPR-Cas systems, e.g., Cas9-gRNA RNP, gRNA, Cas9 mRNA, are described in WO2019067992 and WO2019067910, both incorporated by reference, and are useful for delivery of circular polyribonucleotides and linear polyribonucleotides described herein.

Additional specific LNP formulations useful for delivery of nucleic acids (e.g., circular polyribonucleotides, linear polyribonucleotides) are described in U.S. Pat. Nos. 8,158,601 and 8,168,775, both incorporated by reference, which include formulations used in patisiran, sold under the name ONPATTRO.

In embodiments, a polyribonucleotide (e.g., a circular polyribonucleotide, a linear polyribonucleotide) encoding at least a portion (e.g., an antigenic portion) of a protein or polypeptide described herein is formulated in an LNP, wherein: (a) the LNPs include a cationic lipid, a neutral lipid, a cholesterol, and a PEG lipid, (b) the LNPs have a mean particle size of between 80 nm and 160 nm, and (c) the polyribonucleotide. In embodiments, the polyribonucleotide (e.g., circular polyribonucleotide, linear polyribonucleotide) formulated in an LNP is a vaccine.

Exemplary dosing of polyribonucleotide (e.g., a circular polyribonucleotide, a linear polyribonucleotide) LNP may include about 0.1, 0.25, 0.3, 0.5, 1, 2, 3, 4, 5, 6, 8, 10, or 100 mg/kg (RNA). In some embodiments, a dose of a polyribonucleotide (e.g., a circular polyribonucleotide, a linear polyribonucleotide) antigenic composition described herein is between 30-200 mcg, e.g., 30 mcg, 50 mcg, 75 mcg, 100 mcg, 150 mcg, or 200 mcg.

### Kits

In some aspects, the disclosure provides a kit. In some embodiments, the kit includes (a) a circular polyribonucleotide or a pharmaceutical composition described herein, and, optionally (b) informational material. In some embodiments, the circular polyribonucleotide or pharmaceutical composition may be part of a defined dosing regimen. The informational material may be descriptive, instructional, marketing or other material that relates to the methods described herein and/or the use of the pharmaceutical composition or circular polyribonucleotide for the methods described herein. The pharmaceutical composition or circular polyribonucleotide may comprise material for a single administration (e.g., single dosage form), or may comprise material for multiple administrations (e.g., a “multidose” kit).

The informational material of the kits is not limited in its form. In one embodiment, the informational material may include information about production of the pharmaceutical composition, the pharmaceutical drug substance, or the pharmaceutical drug product, molecular weight of the pharmaceutical composition, the pharmaceutical drug substance, or the pharmaceutical drug product, concentration, date of expiration, batch or production site information, and so forth. In one embodiment, the informational material relates to methods for administering a dosage form of the pharmaceutical composition. In one embodiment, the informational material relates to methods for administering a dosage form of the circular polyribonucleotide.

In addition to a dosage form of the pharmaceutical composition and circular polyribonucleotide described herein, the kit may include other ingredients, such as a solvent or buffer, a stabilizer, a preservative, a flavoring agent (e.g., a bitter antagonist or a sweetener), a fragrance, a dye or coloring agent, for example, to tint or color one or more components in the kit, or other cosmetic ingredient, and/or a second agent for treating a condition or disorder described herein. Alternatively, the other ingredients may be included in the kit, but in different compositions or containers than a pharmaceutical composition or circular polyribonucleotide described herein. In such embodiments, the kit may include instructions for admixing a pharmaceutical composition or nucleic acid molecule (e.g., a circular polyribonucleotide) described herein and the other ingredients, or for using a pharmaceutical composition or nucleic acid molecule (e.g., a circular polyribonucleotide) described herein together with the other ingredients.

In some embodiments, the components of the kit are stored under inert conditions (e.g., under Nitrogen or another inert gas such as Argon). In some embodiments, the components of the kit are stored under anhydrous conditions (e.g., with a desiccant). In some embodiments, the components are stored in a light blocking container such as an amber vial.

A dosage form of a pharmaceutical composition or nucleic acid molecule (e.g., a circular polyribonucleotide) described herein may be provided in any form, e.g., liquid, dried or lyophilized form. It is preferred that a pharmaceutical composition or nucleic acid molecule (e.g., a circular polyribonucleotide) described herein be substantially pure and/or sterile. When a pharmaceutical composition or nucleic acid molecule (e.g., a circular polyribonucleotide) described herein is provided in a liquid solution, the liquid solution preferably is an aqueous solution, with a sterile aqueous solution being preferred. When a pharmaceutical composition or nucleic acid molecule (e.g., a circular polyribonucleotide) described herein is provided as a dried form, reconstitution generally is by the addition of a suitable solvent. The solvent, e.g., sterile water or buffer, can optionally be provided in the kit.

The kit may include one or more containers for the composition containing a dosage form described herein. In some embodiments, the kit contains separate containers, dividers or compartments for the composition and informational material. For example, the pharmaceutical composition or circular polyribonucleotide may be contained in a bottle, vial, or syringe, and the informational material may be contained in a plastic sleeve or packet. In other embodiments, the separate elements of the kit are contained within a single, undivided container. For example, the dosage form of a pharmaceutical composition or nucleic acid molecule (e.g., a circular polyribonucleotide) described herein is contained in a bottle, vial or syringe that has attached thereto the informational material in the form of a label. In some embodiments, the kit includes a plurality (e.g., a pack) of individual containers, each containing one or more unit dosage forms of a pharmaceutical composition or circular polyribonucleotide described herein. For example, the kit includes a plurality of syringes, ampules, foil packets, or blister packs, each containing a single unit dose of a dosage form described herein.

The containers of the kits can be airtight, waterproof (e.g., impermeable to changes in moisture or evaporation), and/or light tight.

The kit optionally includes a device suitable for use of the dosage form, e.g., a syringe, pipette, forceps, measured spoon, swab (e.g., a cotton swab or wooden swab), or any such device.

The kits of the invention may include dosage forms of varying strengths to provide a subject with doses suitable for one or more of the initiation phase regimens, induction phase regimens, or maintenance phase regimens described herein. Alternatively, the kit may include a scored tablet to allow the user to administered divided doses, as needed.

### EXAMPLES

The following examples are put forth so as to provide those of ordinary skill in the art with a description of how the compositions and methods described herein may be used, made, and evaluated, and are intended to be purely exemplary of the disclosure and are not intended to limit the scope of what the inventors regard as their invention.

### Example 1. Purification of Circular Polyribonucleotides

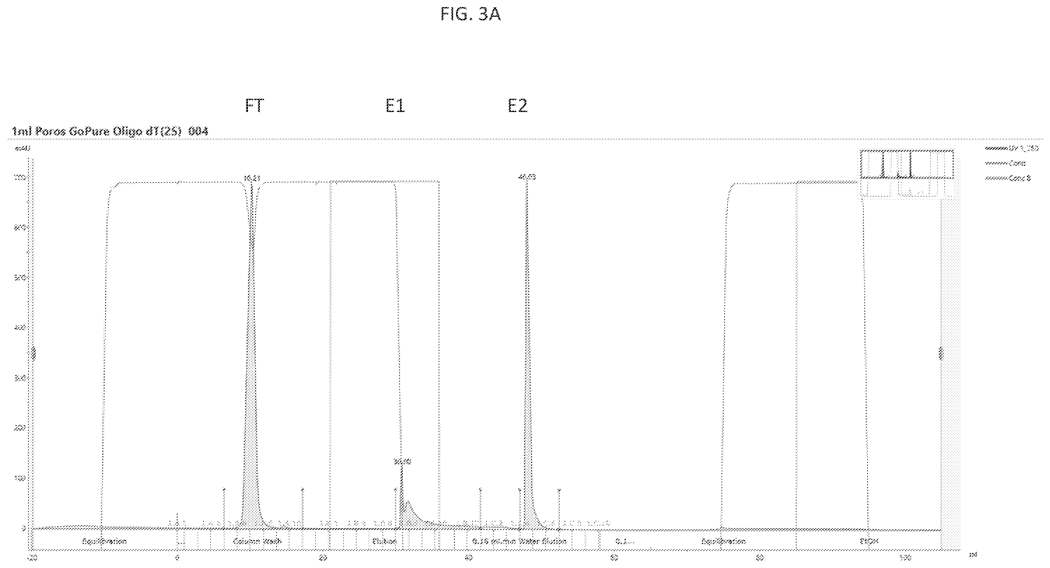

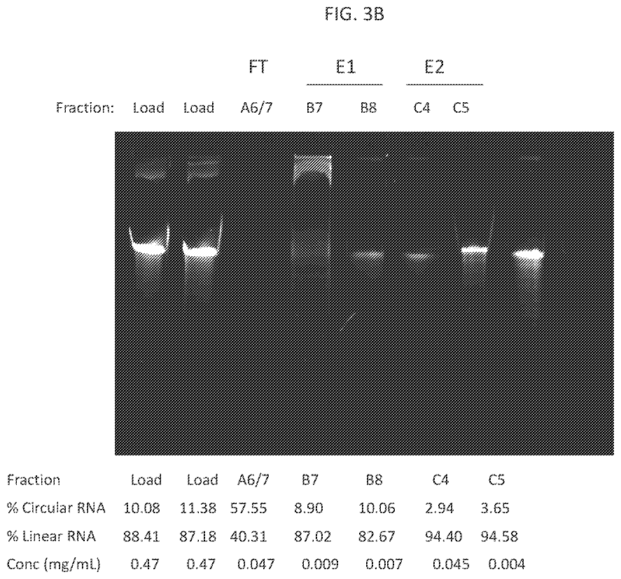

In vitro translation generated a mixture of circRNA and linear RNA. The mixture was purified using an RNA purification column and buffer was exchanged for nuclease-free water using a 10 K Amicon spin concentration device. The material was then polyadenylated using yeast PolyA polymerase according to manufacturer specifications. The sample was adjusted to 0.5M NaCl within 10 mM Tris-HCl, 1 mM EDTA, pH 7.4 and 0.18 mg was loaded onto a column that was washed and equilibrated with 10 mM Tris-HCl, 0.5 M NaCl, 1 mM EDTA, pH 7.4. These results are shown in FIGS. 3A and 3B. The first lane is the sample mixture of IVT-generated circRNA and linRNA that was initially purified and polyadenylated; the second lane is an analysis of the column's flow-through (FT); the third and fourth lanes are an analysis of elution peak E1 (including fractions B7 and B8); and the fifth and sixth lanes are an analysis of peak E2 (including fractions C4 and C5). The sample was eluted with 10 mM Tris-HCl, 1 mM EDTA, pH 7.4 and a peak was observed as denoted as E1 (including fractions B7 and B8) in FIG. 3A. The remainder of the material was then eluted using H₂O and a second elution peak (E2 containing factions C4 and C5) was observed. The percent purity was calculated by SDS PAGE analysis and is shown in FIG. 3B. The load contained 11.4% circRNA and 87.2 linear RNA. The FT pool contained 57.6% circRNA and 40.3% linear RNA. Elution peaks E1 and E2 contained 8-9-10.1% and 2.9-3.7% circRNA and 82.7-87.0 and 94.4-94.6% linear RNA respectively.

### Example 2: Linear RNA Pull Down Specifically Captures Linear RNA Byproducts and Enriches Circular RNA

This example demonstrates enrichment of circular RNA by capturing linear byproducts via polyA-oligo dT interactions.

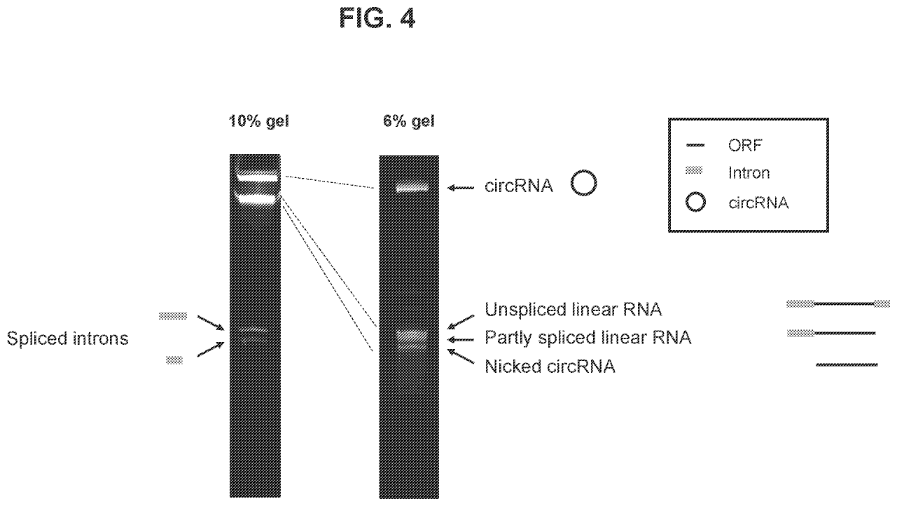

In this example, the construct was designed to have a 3′ half of a catalytic intron, an exon fragment 2 (E2), a polyribonucleotide cargo including an ORF, an exon fragment 1 (E1), and a 5′ half of a catalytic intron. Circular RNAs were generated by self-splicing using a method described herein. In vitro translation generated a mixture of circular RNA and linear RNA byproducts (FIG. 4). The mixture was purified using an RNA purification column and eluted into nuclease-free water. The resulting material was then polyadenylated using E. coli PolyA polymerase according to manufacturer specifications before a second column purification step was performed.

In a first experiment, a 20 μg sample of polyadenylated RNA containing a mixture of circular RNA and linear RNA byproducts was processed using the Oligo(dT)25 Dynabead magnetic beads (ThermoFisher) in duplicate (Run A and Run B). Briefly, the RNA sample was added to a binding buffer (20 mM Tris-HCl, 1.0M LiCl, 2.0 mM EDTA) and incubated with Oligo(dT)25 magnetic beads at room temperature to selectively bind polyadenylated linear species. Application of a magnetic field and removal of the supernatant generated the oligo flowthrough (FT) volume. The retained magnetic beads were washed twice with a wash buffer (10 mM Tris-HCl, 0.15M LiCl, 1.0 mM EDTA) before eluting bound RNA twice with nuclease-free water.

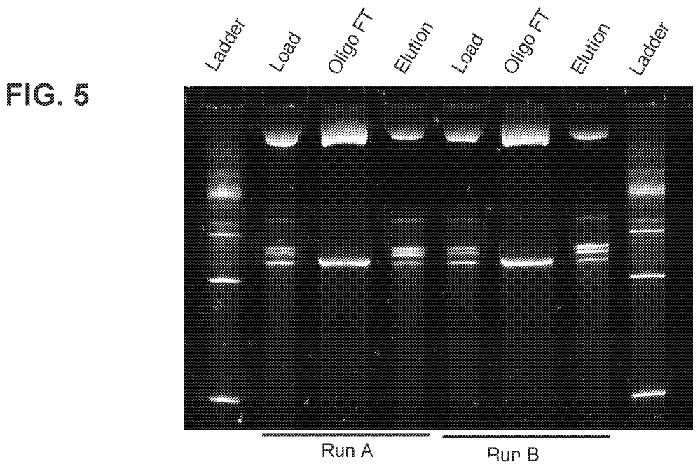

The percent purity was calculated by AEX-HPLC analysis, and the results are shown in Table 1 and a complimentary SDS PAGE gel image (FIG. 5). The load contained 42.35-43.27% circRNA and 56.73-57.65% linear RNA byproducts. The FT pool contained 68.25-68.78% circRNA and 31.75-31.22% linear RNA byproducts. Elution peaks E1 and E2 contained 17.53-20.44% circRNA and 79.56-82.47% linear RNA byproducts.

The concentration of eluted circular RNA and linear RNA byproducts was measured using a spectrophotometer (NanoDrop™ spectrophotometer A260, ThermoFisher) and is shown in Table 1.

TABLE 1
Mass Balance - Recovery

| | | Mass (μg) | | | Recovery (%) | |
|---|---|---|---|---|---|---|
| | Purity (%) | | | linear RNA | | linear RNA |
| | circRNA | RNA | circRNA | byproducts | circRNA | byproducts |
| Load | 42.35-43.2 | 19800 | 8385.3 | 11414.7 | | |
| Flowthrough | 68.25-68.78 | 7120 | 4897.1 | 2222.9 | 58.40 | 19.47 |
| (FT) | | | | | | |
| Elution 1 (E1) | 17.53-20.44 | 6764 | 1382.6 | 5381.4 | 16.48 | 47.14 |
| Elution 2 (E2) | 17.53-20.44 | 2340 | 468.0 | 1872 | 5.58 | 16.93 |

| | | |
|---|---|---|
| Total Recovery | 80.47 | 83.02 |

The results of this first experiment show that oligo(dT)25 beads selectively bind polyadenylated linear RNA byproducts released in the elution volume.

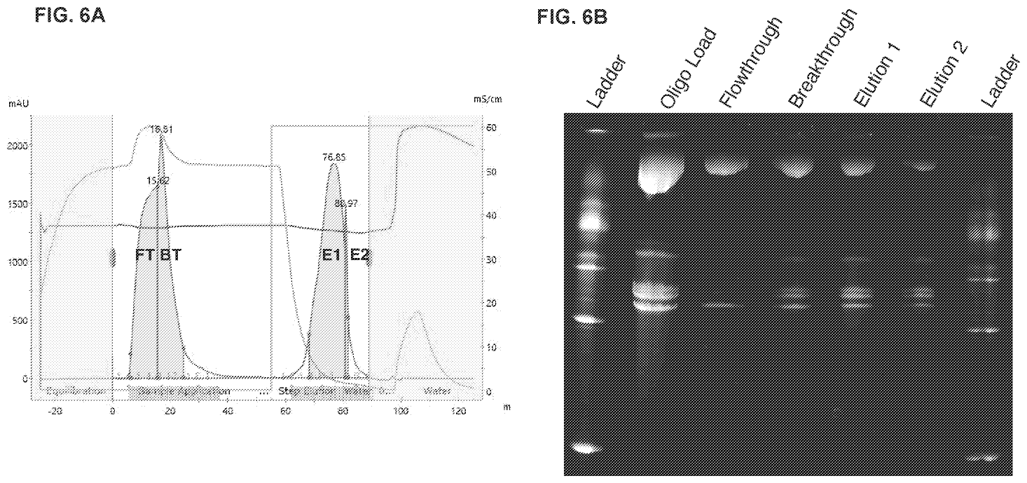

In a second experiment, a 1 mL oligo(dT) column was first washed and equilibrated with 20 mM Tris-HCl, 0.5M NaCl, 10.0 mM EDTA, pH 7.4. The sample was adjusted to 0.5M NaCl within 20 mM Tris-HCl, 10 mM EDTA, pH 7.4, and 8.7 mg was loaded onto the oligo column at a flowrate of 5 mL/min. RNA containing fractions of 0.5 mL were collected throughout the run. Two distinct peaks were observed during the sample application phase and denoted as flowthrough (FT) and breakthrough (BT), respectively, in FIG. 6A.

The sample was eluted with 10 mM Tris-HCl, 1 mM EDTA, pH 8, and a peak was observed and denoted as elution 1 (E1) in FIG. 6A. The remainder of the material was then eluted using water, and a fourth elution peak (E2) was observed (FIG. 6A).

The percent purity was calculated by AEX-HPLC analysis, and the results are shown in Table 2 and a complimentary SDS PAGE gel image (FIG. 6B). The load contained 44.55% circRNA and 55.45% linear RNA byproducts. The FT pool contained 76.46% circRNA and 23.54% linear RNA byproducts. The BT pool contained 59.0% circRNA and 41.0% linear RNA byproducts. Elution peaks E1 and E2 contained 29.14% and 15.52% circRNA and 70.86% and 84.48% linear RNA byproducts, respectively.

The concentration of eluted circular RNA and linear RNA byproducts was measured using a spectrophotometer (NanoDrop™ spectrophotometer A260, ThermoFisher) and is shown in Table 2.

TABLE 2
Mass Balance - Recovery

| | | Mass (μg) | | | Recovery (%) | |
|---|---|---|---|---|---|---|
| | Purity (%) | | | linear RNA | | linear RNA |
| | circRNA | RNA | circRNA | byproducts | circRNA | byproducts |
| Load | 44.55 | 9120 | 4063.0 | 5057.0 | | |
| Flow- | 76.48 | 2572.6 | 1967.0 | 605.6 | 48.41 | 11.98 |
| Through | | | | | | |
| (FT) | | | | | | |
| Break- | 59.00 | 1867.5 | 1101.8 | 765.7 | 27.12 | 15.14 |
| Through | | | | | | |
| (BT) | | | | | | |
| Elution 1 | 29.14 | 3546.25 | 1033.4 | 2512.9 | 25.43 | 49.69 |
| (E1) | | | | | | |
| Elution 2 | 15.52 | 142.7 | 22.1 | 120.6 | 0.54 | 2.38 |
| (E2) | | | | | | |

| | | |
|---|---|---|
| Total Recovery | 101.51 | 79.19 |

### OTHER EMBODIMENTS

While the invention has been described in connection with specific embodiments thereof, it will be understood that it is capable of further modifications and this application is intended to cover any variations, uses, or adaptations of the invention following, in general, the principles of the invention and including such departures from the invention that come within known or customary practice within the art to which the invention pertains and may be applied to the essential features hereinbefore set forth, and follows in the scope of the claims. Other embodiments are within the claims.

1. A method of separating a linear polyribonucleotide from a plurality of polyribonucleotides comprising a mixture of linear polyribonucleotides and circular polyribonucleotides comprising an open reading frame (ORF) encoding a polypeptide, the method comprising:
(a) providing a sample comprising the plurality of polyribonucleotides, wherein a subset of the plurality of polyribonucleotides comprise the linear polyribonucleotide;
(b) attaching a target region to the linear polyribonucleotide;
(c) contacting the sample with an oligonucleotide that hybridizes to the target region; and
(d) separating the linear polyribonucleotide comprising the target region that is hybridized to the oligonucleotide from the plurality of polyribonucleotides in the sample.

2. The method of claim 1, wherein the oligonucleotide is conjugated to a particle.

3. The method of claim 2, wherein the particle comprises a magnetic bead.

4. The method of claim 2, wherein the oligonucleotide is conjugated to a resin comprising a plurality of the particles.

5. The method of claim 4, wherein the resin comprises cross-linked poly[styrene-divinylbenzene], agarose, or SEPHAROSE® agarose.

6. The method of claim 4 or 5, wherein a column comprises the resin.

7. The method of any one of claims 1-6, wherein separating the linear polyribonucleotide comprises immobilizing the oligonucleotide.

8. The method of any one of claims 1-7, wherein separating the linear polyribonucleotide comprises collecting a portion of the sample that is not hybridized to the oligonucleotide.

9. The method of claim 8, wherein the portion of the sample that is not hybridized to the oligonucleotide comprises the circular polyribonucleotide.

10. The method of any one of claims 1-9, wherein a level of expression from the ORF of the circular polyribonucleotide after purification is increased at least 10% relative to a level of expression from the ORF prior to purification.

11. A method of separating a linear polyribonucleotide from a plurality of polyribonucleotides comprising a mixture of linear polyribonucleotides and circular polyribonucleotides, the method comprising:
(a) providing a sample comprising the plurality of polyribonucleotides, wherein a subset of the plurality of polyribonucleotides comprise the linear polyribonucleotide;
(b) attaching a target region to the linear polyribonucleotide;
(c) contacting the sample with a column comprising a resin comprising a plurality of particles conjugated to an oligonucleotide that hybridizes to the target region; and
(d) collecting an eluate comprising a portion of the sample that is not hybridized to the oligonucleotide from the plurality of polyribonucleotides in the sample.

12. The method of claim 10, wherein the portion of the sample that is not hybridized to the oligonucleotide comprises the circular polyribonucleotide.

13. The method of claim 11 or 12, wherein the resin comprises cross-linked poly[styrene-divinylbenzene], agarose, or SEPHAROSE® agarose.

14. The method of any one of claims 1-12, wherein the method comprises a step of circularizing the circular polyribonucleotide from a linear precursor prior to step (a).

15. The method of claim 13, wherein the linear precursor comprises a 5′ self-splicing intron fragment and a 3′ self-splicing intron fragment, and wherein the circular polyribonucleotide is produced by self-splicing of the linear precursor.

16. The method of claim 15, wherein the 5′ self-splicing intron fragment and the 3′ self-splicing intron fragment are each a Group I or Group II self-splicing intron fragment.

17. A method of separating a linear polyribonucleotide from a plurality of polyribonucleotides comprising a mixture of linear polyribonucleotides and circular polyribonucleotides, the method comprising:
(a) circularizing a linear precursor to form the circular polyribonucleotide;
(b) providing a sample comprising the plurality of polyribonucleotides, wherein a subset of the plurality of polyribonucleotides comprise the linear polyribonucleotide;
(c) attaching a target region to the linear polyribonucleotide;
(d) contacting the sample with an oligonucleotide that hybridizes to the target region; and
(e) separating the linear polyribonucleotide comprising the target region that is hybridized to the oligonucleotide from the plurality of polyribonucleotides in the sample.

18. The method of any one of claims 14-17, wherein circularizing the circular polyribonucleotide is produced by splint-ligation of the linear precursor.

19. The method of claim 18, wherein the linear precursor comprises a 5′ self-splicing intron fragment and a 3′ self-splicing intron fragment, and wherein the circular polyribonucleotide is produced by self-splicing of the linear precursor.

20. The method of claim 19, wherein the 5′ self-splicing intron fragment and the 3′ self-splicing intron fragment are each a Group I or Group II self-splicing intron fragment.

21. The method of any one of claims 11-20, wherein the circular polyribonucleotide comprises an ORF.

22. The method of any one of claim 21, wherein the ORF encodes a polypeptide.

23. The method of any one of claims 1-22, wherein the circular polyribonucleotide comprises an internal ribosome entry site (IRES).

24. The method of claim 23, wherein the ORF is operably linked to the IRES.

25. The method of anyone of claims 1-24, wherein the method comprises attaching the target region to a 3′ or 5′ terminus of the linear polyribonucleotide.

26. The method of claim 25, wherein the method comprises attaching the target region to the 3′ terminus of the linear polyribonucleotide.

27. The method of claim 26, wherein the attaching step comprises polyadenylating the 3′ terminus of the linear polyribonucleotide.

28. The method of claim 27, wherein polyadenylating comprises providing a polyA polymerase.

29. The method of claim 26, wherein the attaching step comprises ligating the target region to the 3′ terminus of the linear polyribonucleotide.

30. The method of any one of claims 1-29, wherein the circular polyribonucleotide does not comprise a polyA sequence.

31. The method of claim 25, wherein the circular polyribonucleotide does not comprise a polyA sequence comprising at least 10 contiguous adenosine nucleotides.

32. The method of any one of claims 1-31, wherein the linear polyribonucleotide of step (a) comprises the target region.

33. The method of any one of claims 1-32, wherein the target region comprises a polyA sequence.

34. The method of claim 33, wherein the polyA sequence comprises at least 10 contiguous adenosine nucleotides.

35. The method of claim 33 or 34, wherein the oligonucleotide comprises a polyU or polydT sequence.

36. The method of claim 35, wherein the polydT comprises (dT)₂₅.

37. The method of any one of claims 17-36, wherein the oligonucleotide is conjugated to a particle.

38. The method of claim 37, wherein the particle comprises a magnetic bead.

39. The method of claim 37, wherein the oligonucleotide is conjugated to a resin comprising a plurality of the particles.

40. The method of claim 39, wherein a column comprises the resin.

41. The method of claim 40, wherein the resin comprises cross-linked poly[styrene-divinylbenzene], agarose, or SEPHAROSE® agarose.

42. The method of any one of claims 17-41, wherein separating the linear polyribonucleotide comprises immobilizing the oligonucleotide.

43. The method of any one of claims 17-42, wherein separating the linear polyribonucleotide comprises collecting a portion of the sample that is not hybridized to the oligonucleotide.

44. The method of claim 43, wherein the portion of the sample that is not hybridized to the oligonucleotide comprises the circular polyribonucleotide.

45. The method of any one of claims 1-44, further comprising washing the linear polyribonucleotide comprising the target region that is hybridized to the oligonucleotide one or more times.

46. The method of any one of claims 1-45, further comprising eluting the linear polyribonucleotide comprising the target region from the oligonucleotide.

47. The method of any one of claims 1-46, wherein the method comprises providing a plurality of oligonucleotides, wherein each oligonucleotide hybridizes to a distinct target region.

48. The method of any one of claims 1-47, wherein the oligonucleotide has at least 80% complementarity an equal length portion of the target region.

49. The method of claim 48, wherein the oligonucleotide has at least 85%, 90%, 95%, 97%, 99%, or 100% complementarity to the equal length portion of the target region.

50. The method of any one of claims 1-49, wherein the method comprises providing the oligonucleotide at a molar ratio of 10:1 to 1:10 to the linear polyribonucleotide comprising the target region.

51. The method of any one of claims 11-50, wherein a level of expression from the ORF of the circular polyribonucleotide after purification is increased at least 10% relative to a level of expression from the ORF prior to purification.

52. The method of any one of claims 1-51, wherein the method separates at least 500 μg of the linear polyribonucleotide comprising the target region.

53. The method of claim 52, wherein the method separates from 500 μg to 1000 mg of the linear polyribonucleotide comprising the target region.

54. A population of polyribonucleotides produced by the method of any one of claims 1-53.

55. The population of polyribonucleotides of claim 54, wherein the population comprises a circular polyribonucleotide lacking the target region and the circular polyribonucleotide comprises at least 40% (mol/mol) of the total polyribonucleotides in the composition.

56. The population of polyribonucleotides of claim 54 or 55, wherein the population comprises less than 40% (mol/mol) linear polyribonucleotides of the total polyribonucleotides in the composition.

57. The population of polyribonucleotides of claim 56, wherein the population comprises less than 30%, 20%, 10%, 5%, or 1% (mol/mol) linear polyribonucleotides of the total polyribonucleotides in the composition.

58. The population of polyribonucleotides of any one of claims 54-57, wherein a total weight of polyribonucleotides in the population of polyribonucleotides at least 500 μg.

59. The population of polyribonucleotides of claim 58, wherein the total weight of polyribonucleotides in the population of polyribonucleotides is from 500 μg to 1000 mg.

60. A pharmaceutical composition comprising the population of polyribonucleotides of any one of claims 54-59 and a diluent, carrier, or excipient.
